(19) United States
(12) Patent Application Publication
(10) Pub. No.: US 2021/0024945 A1
(43) Pub. Date: Jan. 28, 2021

(54) THE FATTY ACID DESATURASE 2 FAMILY IN TOMATO

(71) Applicant: THE BOARD OF TRUSTEES OF THE UNIVERSITY OF ARKANSAS, Little Rock, AR (US)

(72) Inventor: Fiona L. Goggin, Fayetteville, AR (US)

(73) Assignee: THE BOARD OF TRUSTEES OF THE UNIVERSITY OF ARKANSAS, Little Rock, AR (US)

(21) Appl. No.: 16/938,331

(22) Filed: Jul. 24, 2020

Related U.S. Application Data
(60) Provisional application No. 62/877,954, filed on Jul. 24, 2019.

(51) Int. Cl. C12N 15/82; A01H 5/10; A01H 6/82; C12N 9/22

(52) U.S. Cl. CPC C12N 15/8247; A01H 5/10; C12N 2310/20; C12N 9/22; A01H 6/825

(57) ABSTRACT

Provided herein are engineered tomato plants with increased biotic stress tolerance and method of using the same, the engineered tomato plant comprising a non-natural modification that increases the function of a target gene selected from the group consisting of Solanum lycopersicum Fatty Acid Desaturase 2-4 (SlFAD2-4), Solanum lycopersicum Fatty Acid Desaturase 2-5 (SlFAD2-5), Solanum lycopersicum Fatty Acid Desaturase 2-6 (SlFAD2-6), Solanum lycopersicum Fatty Acid Desaturase 2-7 (SlFAD2-7), and Solanum lycopersicum Fatty Acid Desaturase 2-9 (SlFAD2-9), wherein the engineered tomato plant exhibits increased biotic stress tolerance as compared to a tomato plant lacking said modification. Also provided herein are engineered tomato plants with decreased linoleic acid production and methods of using the same, the engineered tomato plant comprising a non-natural modification that decreases or silences the function of a target gene selected from the group consisting of Solanum lycopersicum Fatty Acid Desaturase 2-1 (SlFAD2-1), and Solanum lycopersicum Fatty Acid Desaturase 2-2 (SlFAD2-2), wherein the engineered tomato plant exhibits decreased linoleic acid production compared to a tomato plant lacking said modification.

### CROSS-REFERENCE TO RELATED APPLICATIONS

This patent application claims the benefit of priority of U.S. Provisional Patent Application No. 62/877,954, filed Jul. 24, 2019, which is incorporated herein by reference in its entirety.

### STATEMENT REGARDING FEDERALLY SPONSORED RESEARCH

This invention was made with government support under grant number 2015-67013-23412 awarded by the United States Department of Agriculture National Institute of Food and Agriculture. The government has certain rights in the invention.

### SEQUENCE LISTING

A Sequence Listing accompanies this application and is submitted as an ASCII text file of the sequence listing named “169946_00555_ST25.txt” which is 63.7 KB in size and was created on Jul. 24, 2020. The sequence listing is electronically submitted via EFS-Web with the application and is incorporated herein by reference in its entirety.

### BACKGROUND

Nearly 38 million tons of processing tomatoes were produced worldwide in 2017 (WPTC, 2019), and the US crop that year was valued at over 900 million dollars (USDA NASS, 2018). Tomato seeds represent a costly waste product of processing tomatoes, representing about 10% of fruit weight and approximately 60% of processing waste, and the use of tomato seeds for oil production has been proposed as a means of increasing the sustainability and profitability of tomato production (Schieber et al. 2001; Eller et al. 2010). Tomato seed oil is reported to be similar in taste to olive oil (Yilmaz et al. 2015), and it is also marketed as a beauty product because it contains lycopene, ß-carotene, α-tocopherol, and other antioxidants (Eller et al. 2010; Zuorro et al. 2012). However, the composition of tomato seed oil is problematic from the perspective of oxidative stability, which is important to shelf life, as well as from the perspective of stability at high temperatures, which is important for cooking oils or industrial applications.

Accordingly, a need exists in the field for stable tomato seed oils with longer shelf lives as well as engineered tomato lines with improved properties.

### SUMMARY OF THE INVENTION

Engineered tomato plants with increased biotic stress tolerance and methods of using the same eare provided herein. The engineered tomato plants include a non-natural modification that increases the function of a target gene selected from the group consisting of Solanum lycopersicum Fatty Acid Desaturase 2-3 (SlFAD2-3), Solanum lycopersicum Fatty Acid Desaturase 2-4 (SlFAD2-4), Solanum lycopersicum Fatty Acid Desaturase 2-5 (SlFAD2-5), Solanum lycopersicum Fatty Acid Desaturase 2-6 (SlFAD2-6), Solanum lycopersicum Fatty Acid Desaturase 2-7 (SlFAD2-7), and Solanum lycopersicum Fatty Acid Desaturase 2-9 (SlFAD2-9). The engineered tomato plants exhibit increased biotic stress tolerance as compared to a tomato plant lacking said modification. The genes and examples of modifications to increase the function of these genes are provided.

Engineered tomato plants with decreased polyunsaturated fatty acid production are also provided herein. These engineered tomato plants include a non-natural modification that decreases or eliminates the function of a target gene selected from the group consisting of Solanum lycopersicum Fatty Acid Desaturase 2-1 (SlFAD2-1), Solanum lycopersicum Fatty Acid Desaturase 2-2 (SlFAD2-2), and combinations thereof. The engineered tomato plants exhibit decreased polyunsaturated fatty acid production, increased monounsaturated fatty acid production, increased heat tolerance, or combinations thereof as compared to a tomato plant lacking said modification. These plants may be modified to alter the target gene itself or by silencing the target gene.

### BRIEF DESCRIPTION OF DRAWINGS

The patent or patent application file contains at least one drawing in color. Copies of this patent or patent application publication with color drawings will be provided by the Office upon request and payment of the necessary fee.

FIG. 1 shows a comparison of the amino acid sequences encoded by the FAD2 family genes in tomato to Arabidopsis FAD2 and FADE. Nine homologs of the Arabidopsis FAD2 gene (AtFAD2, At3g12120; SEQ ID NO:55) were identified in the tomato genome: SlFAD2-1 (Solyc01g006430; SEQ ID NO:56), SlFAD2-2 (Solyc03g058430; SEQ ID NO:57), SlFAD2-3 (Solyc04g040120; SEQ ID NO:58), SlFAD2-4 (Solyc04g040130; SEQ ID NO:59), SlFAD2-5 (Solyc12g036520; SEQ ID NO:60), SlFAD2-6 (Solyc12g044950; SEQ ID NO:61), SlFAD2-7 (Solyc12g049030; SEQ ID NO:62), SlFAD2-8 (Solyc12g100230; SEQ ID NO:63), and SlFAD2-9 (Solyc12g100250; SEQ ID NO:64). An alignment of their predicted amino acid sequences was performed in ClustalW, and the Arabidopsis FADE gene (AtFAD6, At4g30950; SEQ ID NO:65) and its homolog in tomato (SlFAD6, Solyc07g005510; SEQ ID NO:66) were included for comparison. Ordered from top to bottom, the sequences shown are: SEQ ID NO:58, SEQ ID NO:59, SEQ ID NO:57, SEQ ID NO:55, SEQ ID NO:56, SEQ ID NO:60, SEQ ID NO:63, SEQ ID NO:62, SEQ ID NO:64, SEQ ID NO:61, SEQ ID NO:66, and SEQ ID NO:65. Histidine boxes are highlighted in gray, and ER retrieval motifs are highlighted in blue. Putative transmembrane domains calculated by TMPred are underlined. Residues highlighted in yellow were previously demonstrated to contribute to catalytic differences between canonical FAD2 Δ-12 desaturases and divergent FAD2 hydroxylases (Broun et al. 1998b). Residues highlighted in green are variable between and divergent FAD2 acetylenases (Gagne et al. 2009).

FIGS. 2A-2B show a comparison of FAD2 family members in tomato. A phylogenetic tree based on predicted amino acid sequences was assembled in ClustalW for the nine members of the SlFAD2 gene family in tomato, and AtFAD2, AtFAD6, and SlFAD6 were included for comparison (FIG. 2A). The tree was inferred using the nearest-neighbor interchange algorithm without bootstrapping, and visualized using MEGA5. Values on branches indicate statistical support estimated using a Shimodaira-Hasegawa-like procedure. Branch length is indicated by the scale bar shown below, which represents the number of nucleotide replacements per site. Publicly available RNAseq libraries were also searched to identify the tissues in which each of the 10 tomato genes (SlFAD2-1 through SlFAD2-9, as well as SlFAD6) are expressed (FIG. 2B).

FIGS. 3A-3F show Δ-12 desaturase activity of SlFAD2 family members expressed in yeast. Each member of the SlFAD2 family in tomato was expressed on S. cerevisiae, which lacks native Δ-12 desaturases, and the fatty acid methyl ester (FAMEs) profiles of the yeast cultures were analyzed by GC-MS to determine if heterologous expression of the putative desaturases from tomato would yield C16:2 and C18:2. The vector pYES-DEST52/GUS (FIG. 3A) provides a negative control, and pYES-DEST52/AtFAD2 (FIG. 3B), expressing an Arabidopsis protein with known Δ-12 desaturase activity, is a positive control. Plasmids expressing tomato SlFAD2 family members shown include pYES-DEST52/SlFAD2-1 (FIG. 3C), pYES-DEST52/SlFAD2-2 (FIG. 3D), pYES-DEST52/SlFAD2-4 (FIG. 3E), and pYES-DEST52/SlFAD2-7 (FIG. 3F).

FIG. 3G shows the effects of silencing SlFAD2-1 on the ratio of C18:2 to C18:1 in tomato foliage. A TRV-based virus induced gene silencing (VIGS) system was used to suppress expression of SlFAD2-1 in the spr2 mutant, which accumulates high levels of C18:2. Fatty acid methyl esters (FAMEs) were extracted from the foliage of plants treated with the silencing construct (TRV:SlFAD2-1) or with a vector control (TRV:GUS) and compared using gas chromatography-mass spectrometry. The ratio of C18:2 to C18:1 (expressed in μg/mg fresh weight of tissue) was significantly lower in TRV:SlFAD2-1 than in TRV:GUS (One-way ANOVA, p=0.016). N=4 for the vector control and 6 for TRV:SlFAD2-1. Error bars indicate the standard deviation (SD).

FIGS. 4A-4B show network-based predicted interaction profiles of the nine SlFAD2 genes. FIG. 4A shows visualization of SlFAD2 interconnected network. Each circle represents a SlFAD2 gene, with the size of the circle proportional to the number of genes it interacts with (network degree). Blue lines indicate overlaps between two connected SlFAD2s, with the thickness of the link proportional to the overlap score. FIG. 4B shows a heatmap showing functional terms from the gene ontology biological process (GO BP) categories over-represented in the interaction profile of each SlFAD2 gene. The cells are colored according to the enrichment score (range indicated in the legend). Enrichment scores were calculated as the −log₁₀ (q value), where the q value is the hypergeometric test p value corrected for false discoveries using Benjamini-Hochberg's method. Blue grids indicate no significant overlap between the GO BP term on the y axis and the corresponding SlFAD2 gene on the x axis, whereas yellow to red gradient indicates significant overlaps, with darker colors indicating higher statistical significance.

FIGS. 5A-5D show transcriptional regulation of SlFAD2-4 and SlFAD2-7 in response to salicylic acid and wounding. RT-qPCR was used to measure the relative transcript abundance of SlFAD2-4 (FIG. 5A) and SlFAD2-7 (FIG. 5B) in tomato foliage (cv. Castlemart) treated with 100 μM salicylic acid (SA) or a blank carrier solution (Mock). The effect of mechanical wounding on SlFAD2-4 (FIG. 5C) and SlFAD2-7 (FIG. 5D) expression was also tested in two genetic backgrounds: WT plants (cv. Castlemart) and the spr2 mutant, which is impaired in jasmonate signaling due to a deficiency in C18:3 for jasmonate synthesis. Relative expression was measured 24 hrs after treatment, and expression was normalized relative to RPL2. Error bars indicate the standard error of the means (SEM). Data was analyzed by one-way ANOVA (FIGS. 5A and 5B) or two-way ANOVA (FIGS. 5C and 5D), and treatments labeled with double or triple asterisks are significantly different at α=0.05 and α=0.001 respectively. Comparisons labeled “N.S.” are not significantly different at α=0.10. N=8 for A and B, and n=3 for C and D.

FIGS. 6A-6D show effects of silencing SlFAD2-4 and SlFAD2-7 on aphid infestation. A TRV-based VIGS system was used to suppress expression of SlFAD2-4 in wild-type (WT, cv. Castlemart) tomato plants (FIG. 6A), which are susceptible to the potato aphid, and in the spr2 mutant (FIG. 6B), which is resistant to this aphid species. SlFAD2-7 was also silenced in WT (FIG. 6C) and spr2 (FIG. 6D). Plants were challenged with aphids (3 cages/plant; 4 adults/cage), and offspring numbers were measured at 4 dpi. Error bars indicate the standard error of the means (SEM). Double asterisks represent significant differences at α=0.05, and n.s. indicates no significant differences at α=0.1 (one-way ANOVA). N≥16 plants per treatment group.

FIGS. 7A-7D show identification of fatty acids produced by heterologous SlFAD2 expression in yeast. The presence of 9,12 Hexadecadienoic acid (C16:2^{Δ9,12}) and 9,12-Octadecanoic acid (i.e. linoleic acid, or 18:2^{Δ9,12}) in a yeast line expressing SlFAD2-2 was confirmed by comparing full MS scans at the expected retention times for C16:2^{Δ9,12} (FIG. 7A) and 18:2^{Δ9,12} (FIG. 7C) to mass spectra obtained from the National Institute of Standards and Technology (NIST) Mass Spectral Library (FIGS. 7B and 7D).

### INCORPORATION BY REFERENCE

All publications, patents, and patent applications mentioned in this specification are herein incorporated by reference to the same extent as if each individual publication, patent, and patent application was specifically and individually indicated to be incorporated by reference.

### DETAILED DESCRIPTION OF THE INVENTION

### In General

The present disclosure describes engineered tomato plants with increased or decreased function of a gene selected from the group consisting of Solanum lycopersicum Fatty Acid Desaturase 2-1 (SlFAD2-1), Solanum lycopersicum Fatty Acid Desaturase 2-2 (SlFAD2-2), Solanum lycopersicum Fatty Acid Desaturase 2-3 (SlFAD2-3), Solanum lycopersicum Fatty Acid Desaturase 2-4 (SlFAD2-4), Solanum lycopersicum Fatty Acid Desaturase 2-5 (SlFAD2-5), Solanum lycopersicum Fatty Acid Desaturase 2-6 (SlFAD2-6), Solanum lycopersicum Fatty Acid Desaturase 2-7 (SlFAD2-7), Solanum lycopersicum Fatty Acid Desaturase 2-8 (SlFAD2-8), Solanum lycopersicum Fatty Acid Desaturase 2-9 (SlFAD2-9), Solanum lycopersicum Fatty Acid Desaturase 6 (SlFAD6), and combinations thereof. (See Table 1 for the SEQ ID NOs associated with the cDNA and protein sequences of these genes.) Engineered tomato plants described herein may have increased resistance to biotic or abiotic stress, decreased polyunsaturated fatty acid content, increased monounsaturated fatty acid content, or a combination thereof.

TABLE 1
cDNA and amino acid sequences for the target genes of interest.

| | | Amino acid |
|---|---|---|
| FAD gene | cDNA sequence | sequence |
| SlFAD2-1 (Solyc01g006430.2.1) | SEQ ID NO: 45 | SEQ ID NO: 56 |
| SlFAD2-2 (Solyc03g058430.1.1) | SEQ ID NO: 46 | SEQ ID NO: 57 |
| SlFAD2-3 (Solyc04g040120.1.1) | SEQ ID NO: 47 | SEQ ID NO: 58 |
| SlFAD2-4 (Solyc04g040130.1.1) | SEQ ID NO: 48 | SEQ ID NO: 59 |
| SlFAD2-5 (Solyc12g036520.1.1) | SEQ ID NO: 49 | SEQ ID NO: 60 |
| SlFAD2-6 (Solyc12g044950.1.1) | SEQ ID NO: 50 | SEQ ID NO: 61 |
| SlFAD2-7 (Solyc12g049030.1.1) | SEQ ID NO: 51 | SEQ ID NO: 62 |
| SlFAD2-8 (Solyc12g100230.1.1) | SEQ ID NO: 52 | SEQ ID NO: 63 |
| SlFAD2-9 (Solyc12g100250.1.1) | SEQ ID NO: 53 | SEQ ID NO: 64 |
| SlFAD6 (Solyc07g005510.2.1) | SEQ ID NO: 54 | SEO ID NO: 65 |

Fatty acids are essential components of cellular membranes and storage lipids, and are precursors for a wide variety of plant metabolites including signaling molecules and phytoalexins (Ohlrogge and Browse, 1995; Lim et al. 2017). The relative abundance of different fatty acid species and their derivatives in plants is regulated in part through the action of fatty acid desaturases (FADs) and related enzymes. FADs add double bonds at specific positions within the acyl chain of the fatty acid, thereby shaping many of the chemical properties of the molecule, including its melting temperature (Aguilar and de Mendoza, 2006), its oxidative stability (Shahidi and Zhong, 2010), and its ability to act as a substrate for synthesis of other metabolites (Chehab et al. 2007). Duplication of genes encoding FADs has also in some plant species enabled the functional diversification of these enzymes, giving rise to divergent catalytic capabilities and synthesis of novel fatty acids or their derivatives (Cao et al., 2013). Because of their impacts on the chemical properties of membrane- and storage-lipids, and on the accumulation of secondary metabolites derived from fatty acids, standard and divergent FADs influence many important physiological and agronomic properties of crops. For example, they impact seed oil quality (Haun et al. 2014), fruit aromas (Dominguez et al. 2010), abiotic and biotic stress resistance (Upchurch, 2008).

The most abundant fatty acid in tomato seed oil is the polyunsaturated fatty acid linoleic acid (C18:2), which represents ˜37-57% of its total fatty content (Botinstean et al. 2015). Linoleic acid has relatively low heat stability, is prone to oxidation, and is a major contributor to rancidity in foods (Haman et al. 2017). Therefore, a need in the art exists for tomato cultivars with reduced linoleic acid content and increased oleic acid content. Plant cultivars that are low in linoleic acid and high in its monounsaturated precursor oleic acid may be generated by selecting for or engineering reduced FAD2 activity. In tomato, SlFAD2-1 and SlFAD2-2 are targeted to enhance oil seed quality by increasing oleic acid content while decreasing linoleic acid content.

In some aspects, the present disclosure describes an engineered tomato plant with increased biotic stress tolerance and comprising a non-natural modification that increases the expression and function of a target gene selected from the group consisting of Solanum lycopersicum Fatty Acid Desaturase 2-4 (SlFAD2-4), Solanum lycopersicum Fatty Acid Desaturase 2-5 (SlFAD2-5), Solanum lycopersicum Fatty Acid Desaturase 2-6 (SlFAD2-6), Solanum lycopersicum Fatty Acid Desaturase 2-7 (SlFAD2-7), and Solanum lycopersicum Fatty Acid Desaturase 2-9 (SlFAD2-9). The engineered tomato plant exhibits increased biotic stress tolerance as compared to a tomato plant lacking said modification.

In some aspects, the present disclosure describes an engineered tomato plant with decreased polyunsaturated fatty acid production and comprising a non-natural modification that decreases or silences the function of a target gene selected from the group consisting of Solanum lycopersicum Fatty Acid Desaturase 2-1 (SlFAD2-1), and Solanum lycopersicum Fatty Acid Desaturase 2-2 (SlFAD2-2). The engineered tomato plant exhibits decreased production of linoleic acid and other polyunsaturated fatty acids, increased production of monounsaturated fatty acids such as oleic acid, increased tolerance of stresses such as heat, or combinations thereof compared to a tomato plant lacking said modification.

As used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, and “having” can be used interchangeably.

As used herein, the terms “approximately” or “about” in reference to a number are generally taken to include numbers that fall within a range of 5% in either direction (greater than or less than) the number unless otherwise stated or otherwise evident from the context (except where such number would exceed 100% of a possible value). Where ranges are stated, the endpoints are included within the range unless otherwise stated or otherwise evident from the context.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications and patents specifically mentioned herein are incorporated by reference for all purposes including describing and disclosing the chemicals, cell lines, vectors, animals, instruments, statistical analysis and methodologies which are reported in the publications which might be used in connection with the invention. All references cited in this specification are to be taken as indicative of the level of skill in the art.

The practice of the present invention will employ, unless otherwise indicated, conventional techniques of molecular biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); and Handbook Of Experimental Immunology, Volumes I-IV (D. M. Weir and C. C. Blackwell, eds., 1986).

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. Promoters that cause a gene to be expressed in most cell types at most times are commonly referred to as “constitutive promoters”. Promoters that allow the selective expression of a gene in most cell types are referred to as “inducible promoters”.

In some embodiments, the promoter is a constitutive tissue-specific promoter. Constitutive tissue-specific promoters suitable for use in tomato include, but are not limited to, Cauliflower Mosaic Virus 35S promoter, the Figwort Mosaic Virus 34S promoter, or the Arabidopsis Ubiquitin10 promoter. See, for example, Somssich et al. (Somssich I E, Bollmann J, Hahlbrock K, Kombrink E, Schulz W (1989) Differential early activation of defense-related genes in elicitor-treated parsley cells. Plant Mol Biol 12: 227-234). In some embodiments, the promoter is an inducible tissue-specific promoter. Inducible tissue-specific promoters suitable for use in tomato include, but are not limited to, stress-responsive synthetic promoters such as 4×RSRE, sab, or 4×GCC. See, for example, Liu et ail (Liu, W., and Stewart, C. N. 2016. Plant synthetic promoters and transcription factors. Current Opinion in Biotechnology 2016, 37:36-44). In some embodiments, for engineering enhanced stress resistance, suitable tissue-specific promoters include, but are not limited to, green tissue-specific promoters such as the CAB promoters or root-specific promoters such as the tomato SlREO promoter. See, for example, Song et al. (Song, G., Honda H., and Yamaguchi K. 2007. Expression of a Rice Chlorophyll a/b Binding Protein Promoter in Sweetpotato. Journal of the American Society for Horticultural Science J. Amer. Soc. Hort. Sci. 132 (4), pp. 551-556) and Jones et al. (Jones, M. O., et al. (2008). The promoter from SlREO, a highly-expressed, root-specific Solanum lycopersicum gene, directs expression to cortex of mature roots. Functional Plant Biology, 35(12), pp. 1224-1233). In some embodiments, for engineering reduced monounsaturated fatty acid content in seed oil, seed-specific promoters are used. Suitable promoters may include regulatory elements from the tomato genome that target expression to seeds and fruit, such as, but not limited to, E273715 and E541096. See, for example, Lim et al. (Lim C. J., Lee H. Y., Kim W. B., Lee B., Kim J., Ahmad R., Kim H. A., Yi S. Y., Hur C., Kwon S. 2012. Screening of tissue-specific genes and promoters in tomato by comparing genome wide expression profiles of Arabidopsis orthologues. Mol Cells. 2012; 34:53-59). The seed-specific promoters from other plant species may be used to drive seed-specific expression including, but not limited to, napin, legumin, or USP promoters. See, for example, Yuan et al. (Yuan, Y., Liang, Y., Gao, L., Sun, R., Zheng, Y., and Li, D. (2015). Functional heterologous expression of a lysophosphatidic acid acyltransferase from coconut (Cocos nucifera L.) endosperm in Saccharomyces cerevisiae and Nicotiana tabacum. Sci. Hortic. 192, 224-230), Fiedler et al. (Fiedler, U., and Conrad, U. (1995). High-level production and long-term storage of engineered antibodies in transgenic tobacco seeds. Biotechnology (N. Y.) 13, 1090-1093), Hornung et al. (Hornung, E., Krueger, C., Pernstich, C., Gipmans, M., Porzel, A., and Feussner, I. (2005). Production of (10E,12Z)-conjugated linoleic acid in yeast and tobacco seeds. Biochim. Biophys. Acta 1738, 105-114), and Weichert et al. (Weichert N, Hauptmann V, Helmold C and Conrad U (2016) Seed-Specific Expression of Spider Silk Protein Multimers Causes Long-Term Stability. Front. Plant Sci. 7:6).

A “host cell” is a cell that has been transformed or transfected, or is capable of transformation or transfection by an exogenous polynucleotide sequence. A host cell that has been transformed or transfected may be more specifically referred to as a “recombinant host cell”.

By “modulation” of the target gene, we mean to include over-expression, increased function, reduced-expression, reduced function, or gene knockout. In some embodiments, the target gene is over-expressed. In some embodiments, expression of the target gene is reduced. In some embodiments, the target gene is knocked out. By “modulation of the gene,” we also mean to include modification or manipulation of the regulatory regions of the target gene.

By “a non-natural modification,” we mean to include all manner of recombinant and transgenic manipulation to the plant. For example, a plant comprising an extra copy of the target gene has a non-natural modification. A plant comprising a vector containing the target gene and a promoter from a different tomato or plant line is a non-natural modification. A plant comprising a vector expressing an miRNA, a dsRNA, an hpRNA, a siRNA, or other RNA associated with RNA interference to silence or down regulate gene expression is a non-natural modification. We also mean to include modification or manipulation of the regulatory regions of the target gene or of any region that is contiguous with the target gene up to 5 KB on either side of the target sequence. Non-natural modification also covers CRISP/Cas9 mediated gene editing of a target gene. Also included are modifications to the target gene such as, but not limited to, insertions, deletions, non-sense mutations, substitutions, etc., that may increase or decrease the expression of the target gene or gene product thereof.

By “engineered plant” or “engineered tomato,” we mean a plant or tomato that includes a non-natural modification as described herein. In some embodiments, the engineered plant comprises an extra copy of the target gene. In some embodiments, the engineered plant comprises a vector containing the target gene and a promoter. In some embodiments, the engineered plant includes a non-natural modification or manipulation of the regulator region of the target gene or of any region that is contiguous with the target gene up to 5 KB on either side of the target gene sequence. In some embodiments, the engineered plant comprises a vector expressing an RNA associated with RNA interference to silence or down regulate gene expression. In some embodiments, the engineered plant comprises an RNA selected from the group consisting of miRNA, dsRNA, hpRNA, or siRNA specific to or complementary to the target gene, whereby said RNA downregulates or silences the gene. In some embodiments, a target gene of the engineered plant has been modified using CRISPR/Cas9 mediated gene editing. In some embodiments, a target gene of the engineered plant has been modified with an insertion, deletion, non-sense mutation, substitution, etc., that increases or decreases the expression of the target gene product thereof.

By “transgenic plant” or “transgenic tomato,” we mean a plant or tomato including one or more copies of a transgene. The transgene may be the target gene of interest or another gene or nucleic acid sequence that regulates the expression and activity of the target gene. For example, a gene encoding a transcription factor or a promoter sequence.

A polypeptide “substantially identical” to a comparative polypeptide varies from the comparative polypeptide, but has at least 80%, preferably at least 85%, more preferably at least 90%, and yet more preferably at least 95% sequence identity at the amino acid level over the complete amino acid sequence, and retains substantially the same biological function as the corresponding polypeptide to which comparison is made.

The term “substantial sequence homology” refers to DNA or RNA sequences that have de minimus sequence variations from, and retain substantially the same biological functions as the corresponding sequences to which comparison is made.

As used herein, “hybridizes under stringent conditions” is intended to describe conditions for hybridization and washing under which nucleotide sequences that are significantly identical or homologous to each other remain hybridized to each other. Such stringent conditions are known to those skilled in the art and can be found in Current Protocols in Molecular Biology, Ausubel et al., eds., John Wiley & Sons, Inc. (1995), sections 2, 4 and 6. Additional stringent conditions can be found in Molecular Cloning: A Laboratory Manual, Sambrook et al., Cold Spring Harbor Press, Cold Spring Harbor, N.Y. (1989), chapters 7, 9 and 11. A preferred, non-limiting example of stringent hybridization conditions includes hybridization in 4× sodium chlorine/sodium citrate (SSC), at about 65-70° C. (or hybridization in 4×SSC plus 50% formamide at about 42-50° C.) followed by one or more washes in 1×SSC, at about 65-70° C. A preferred, non-limiting example of highly stringent hybridization conditions includes hybridization in 1×SSC, at about 65-70° C. (or hybridization in 4×SSC plus 50% formamide at about 42-50° C.) followed by one or more washes in 0.3×SSC, at about 65-70° C. A preferred, non-limiting example of highly stringent hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Ranges intermediate to the above-recited values, e.g., at 65-70° C. or at 42-50° C. are also intended to be encompassed by the present invention. SSPE (1×SSPE is 0.15 M NaCl, 10 mM NaH₂PO₄, and 1.25 mM EDTA, pH 7.4) can be substituted for SSC (1×SSPE is 0.15 M NaCl and 15 mM sodium citrate) in the hybridization and wash buffers; washes are performed for 15 minutes each after hybridization is complete. The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ (° C.)=2(# of A+T bases)+4(# of G+C bases). For hybrids between 18 and 49 base pairs in length, Tₘ (° C.)=81.5+16.6(log₁₀[Na+])+0.41(% G+C)−(600/N), where N is the number of bases in the hybrid, and [Na+] is the concentration of sodium ions in the hybridization buffer ([Na+] for 1×SSC=0.165 M). It will also be recognized by the skilled practitioner that additional reagents may be added to the hybridization and/or wash buffers to decrease non-specific hybridization of nucleic acid molecules to membranes, for example, nitrocellulose or nylon membranes, including but not limited to blocking agents (e.g., BSA or salmon or herring sperm carrier DNA), detergents (e.g., SDS) chelating agents (e.g., EDTA), Ficoll, PVP and the like. When using nylon membranes, in particular, an additional preferred, non-limiting example of stringent hybridization conditions is hybridization in 0.25-0.5M NaH₂PO₄, 7% SDS at about 65° C., followed by one or more washed at 0.02M NaH₂PO₄, 1% SDS at 65° C., see e.g., Church and Gilbert (1984) Proc. Natl. Acad. Sci. USA 81: 1991-1995, (or alternatively 0.2×SSC, 1% SDS).

“Polynucleotide(s)” generally refers to any polyribonucleotide or polydeoxribonucleotide, which may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. As used herein, the term “polynucleotide(s)” also includes DNAs or RNAs as described above that contain one or more modified bases. Thus, DNAs or RNAs with backbones modified for stability or for other reasons are “polynucleotide(s)” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. “Polynucleotide(s)” also embraces short polynucleotides often referred to as oligonucleotide(s).

The term “isolated nucleic acid” used in the specification and claims means a nucleic acid isolated from its natural environment or prepared using synthetic methods such as those known to one of ordinary skill in the art. Complete purification is not required in either case. The nucleic acids of the invention can be isolated and purified from normally associated material in conventional ways such that in the purified preparation the nucleic acid is the predominant species in the preparation. At the very least, the degree of purification is such that the extraneous material in the preparation does not interfere with use of the nucleic acid of the invention in the manner disclosed herein. The nucleic acid is preferably at least about 85% pure, more preferably at least about 95% pure and most preferably at least about 99% pure.

Further, an isolated nucleic acid has a structure that is not identical to that of any naturally occurring nucleic acid or to that of any fragment of a naturally occurring genomic nucleic acid spanning more than three separate genes. An isolated nucleic acid also includes, without limitation, (a) a nucleic acid having a sequence of a naturally occurring genomic or extrachromosomal nucleic acid molecule but which is not flanked by the coding sequences that flank the sequence in its natural position; (b) a nucleic acid incorporated into a vector or into a prokaryote or eukaryote genome such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene. Specifically excluded from this definition are nucleic acids present in mixtures of clones, e.g., as those occurring in a DNA library such as a cDNA or genomic DNA library. An isolated nucleic acid can be modified or unmodified DNA or RNA, whether fully or partially single-stranded or double-stranded or even triple-stranded. A nucleic acid can be chemically or enzymatically modified and can include so-called non-standard bases such as inosine, as described in a preceding definition.

The term “operably linked” means that the linkage (e.g., DNA segment) between the DNA segments so linked is such that the described effect of one of the linked segments on the other is capable of occurring. “Linked” shall refer to physically adjoined segments and, more broadly, to segments which are spatially contained relative to each other such that the described effect is capable of occurring (e.g., DNA segments may be present on two separate plasmids but contained within a cell such that the described effect is nonetheless achieved). Effecting operable linkages for the various purposes stated herein is well within the skill of those of ordinary skill in the art, particularly with the teaching of the instant specification.

As used herein the term “gene product” shall refer to the biochemical material, either RNA or protein, resulting from expression of a gene.

The term “heterologous” is used for any combination of DNA sequences that is not normally found intimately associated in nature (e.g., a green fluorescent protein (GFP) reporter gene operably linked to a SV40 promoter). A “heterologous gene” shall refer to a gene not naturally present in a host cell (e.g., a luciferase gene present in a retinoblastoma cell line).

As used herein, the term “homolog” refers to a gene related to a second gene by descent from a common ancestral DNA sequence. The term, homolog, may apply to the relationship between genes separated by the event of speciation (i.e., orthologs) or to the relationship between genes separated by the event of genetic duplication (i.e., paralogs). “Orthologs” are genes in different species that evolved from a common ancestral gene by speciation. Normally, orthologs retain the same function in the course of evolution. Identification of orthologs is important for reliable prediction of gene function in newly sequenced genomes. “Paralogs” are genes related by duplication within a genome. Orthologs retain the same function in the course of evolution, whereas paralogs evolve new functions, even if these are related to the original one.

The nucleotides that occur in the various nucleotide sequences appearing herein have their usual single-letter designations (A, G, T, C or U) used routinely in the art. In the present specification and claims, references to Greek letters may either be written out as alpha, beta, etc. or the corresponding Greek letter symbols (e.g., α, β, etc.) may sometimes be used.

Nucleic acid constructs useful in the invention may be prepared in conventional ways, by isolating the desired genes from an appropriate host, by synthesizing all or a portion of the genes, or combinations thereof. Similarly, the regulatory signals, the transcriptional and translational initiation and termination regions, may be isolated from a natural source, be synthesized, or combinations thereof. The various fragments may be subjected to endonuclease digestion (restriction), ligation, sequencing, in vitro mutagenesis, primer repair, or the like. The various manipulations are well known in the literature and will be employed to achieve specific purposes.

The various nucleic acids and/or fragments thereof may be combined, cloned, isolated and sequenced in accordance with conventional ways. After each manipulation, the DNA fragment or combination of fragments may be inserted into a cloning vector, the vector transformed into a cloning host, e.g. Escherichia coli, the cloning host grown up, lysed, the plasmid isolated and the fragment analyzed by restriction analysis, sequencing, combinations thereof, or the like.

Various vectors may be employed during the course of development of the construct and transformation of host cells. Thee vectors may include cloning vectors, expression vectors, and vectors providing for integration into the host or the use of bare DNA for transformation and integration. The cloning vector will be characterized, for the most part, by having a replication original functional in the cloning host, a marker for selection of a host containing the cloning vector, may have one or more polylinkers, or additional sequences for insertion, selection, manipulation, ease of sequencing, excision, or the like. In addition, shuttle vectors may be employed, where the vector may have two or more origins of replication, which allows the vector to be replicated in more than one host, e.g. a prokaryotic host and a eukaryotic host.

Expression vectors will usually provide for insertion of a construct which includes the transcriptional and translational initiation region and termination region or the construct may lack one or both of the regulatory regions, which will be provided by the expression vector upon insertion of the sequence encoding the protein product. Thus, the construct may be inserted into a gene having functional transcriptional and translational regions, where the insertion is proximal to the 5′-terminus of the existing gene and the construct comes under the regulatory control of the existing regulatory regions. Normally, it would be desirable for the initiation codon to be 5′ of the existing initiation codon, unless a fused product is acceptable, or the initiation codon is out of phase with the existing initiation codon. In other instances, expression vectors exist which have one or more restriction sites between the initiation and termination regulatory regions, so that the structural gene may be inserted at the restriction site(s) and be under the regulatory control of these regions.

Suitable methods for plant transformation for use with the current invention are believed to include virtually any method by which DNA can be introduced into a cell, such as by direct delivery of DNA by PEG-mediated transformation of protoplasts, by desiccation/inhibition-mediated DNA uptake by electroporation, by agitation with silicon carbide fibers, by Agrobacterium-mediated transformation, and by acceleration of DNA coated particles. Through the application of techniques such as these, tomato cells, as well as those of virtually any other plant species, may be stably transformed, and these cells developed into engineered plants.

### Suitable Tomato Lines

We envision that the present disclosure would be useful in all tomato varieties and lines known and used in the art. Suitable tomato lines include, but are not limited to, Moneymaker, MicroTom, Castlemart, Ailsa Craig, Trust, Hybrid 882, Yaqui, Monica, Mountain Belle, BHN 444, and other. Suitable tomato lines may include lines used for research purposes or lines and cultivars used in commercial tomato production.

### Increase in Gene Expression

The present disclosure, in certain aspects, includes steps of increasing the function of a target gene to yield a desirable phenotype. To that end, in some embodiments, DNA may be introduced into the plant or plant cell to yield a non-natural modification that enables over-expression or enhanced activity of a target gene selected from the group consisting of SlFAD2-3, SlFAD2-4, SlFAD2-5, SlFAD2-6, SlFAD2-7, SlFAD2-9, and combinations thereof to yield a phenotype with increased function of the recited gene and increased biotic stress tolerance. An increase in function may be an increase of at least 50%, at least 60%, at least 70%, at least 80%, or at least 90% when compared to a plant of the same genetic background without the non-natural modification. An increase in function may be an increase of at least 2-fold, at least 3-fold, at least 4-fold, at least 5-fold, or at least 6-fold when compared to a plant of the same genetic background without the non-natural modification. Increased gene function may be measured as an increased in mRNA transcripts (e.g., as measured by RT-PCR) or increased protein production. An increase in biotic stress tolerance may include an increased resistance to plant pathogens (e.g., bacteria, virus, fungi, oomycetes, viroids, etc.), insects, nematodes, or other pests. Increased biotic stress tolerance may be measured as a reduction of at least 10%, at least 20%, at least 30%, at least 40%, at least 50%, at least 60%, or at least 70% in damage to a plant exposed to mechanical wounding, a pest or a pathogen when compared to a plant of the same genetic background without the non-natural modification. In some embodiments, the non-natural modification to increase expression of SlFAD2-3, SlFAD2-4, SlFAD2-5, SlFAD2-6, SlFAD2-7, SlFAD2-9, and combinations thereof is introduced into a tomato plant and the control plant is a tomato plant of the same genotype without the non-natural mutation.

In some embodiments, DNA may be introduced into the plant or plant cell to enable over expression of a target gene selected from the group consisting of SlFAD2-1, SlFAD2-2, SlFAD6, or combinations thereof to yield a phenotype with increased tolerance of abiotic stresses such as cold or variations in salinity.

The introduction of DNA could include transformation of tomato cells with multiple copies of the gene, use of modified or natural promoters designed to over-express the gene, use of constitutive or tissue-specific promoters designed to focus expression in specific tissues or in a non-specific manner, and use of vectors to carry a copy or copies of the gene. One may also wish to transform plant cells with regulatory elements that will modify the native expression of the target gene or modify existing regulatory elements.

Suitable methods for transformation of plant or other cells for use with the current invention are believed to include virtually any method by which DNA can be introduced into a cell, such as by direct delivery of DNA such as by PEG-mediated transformation of protoplasts. Other suitable tomato and tomato cell transformation methods include, but are not limited to Agrobacterium-mediated transformation and particle bombardment (e.g., acceleration of DNA coated particles). These methods and their use are well known in the art. The most likely transgenic approach would typically be using tissue-specific promoters that are stronger than the endogenous version in the line that is targeted.

After effecting delivery of exogenous DNA to recipient cells, the next steps generally concern identifying the transformed cells for further culturing and plant regeneration. In order to improve the ability to identify transformants, one may desire to employ a selectable or screenable marker gene with a transformation vector prepared in accordance with the disclosure. In this case, one would then generally assay the potentially transformed cell population by exposing the cells to a selective agent or agents or one would screen the cells for the desired marker gene trait. Suitable selectable or screenable markers include, but are not limited to, antibiotic resistance markers (e.g., kananmycin (nptII) and hygromycin (hpt)), herbicide resistance markers (e.g., glyphosate (epsps) and glufosinate ammonium (bar or pat)), carbohydrate metabolism markers (e.g., mannose (manA)), growth regulator markers (e.g., isopentenyl transferase (ipt)), and combinations thereof. See, for example, Breyer et al. (Didier Breyer, Lilya Kopertekh & Dirk Reheul (2014) Alternatives to Antibiotic Resistance Marker Genes for In Vitro Selection of Genetically Modified Plants—Scientific Developments, Current Use, Operational Access and Biosafety Considerations, Critical Reviews in Plant Sciences, 33:4, 286-330).

Cells that survive the exposure to the selective agent, or cells that have been scored positive in a screening assay, may be cultured in media that supports regeneration of plants. In an exemplary embodiment, medium may be modified by including further substances such as growth regulators. Examples of such growth regulators are zeatin, indole-3-acetic acid, benzylaminopurine, indolebutyric acid, kinetin, or naphthaleneacetic acid. See, for example, Bhatia et al. (P. Bhatia, N. Ashwath, T. Senaratna and D. Midmore, Tissue culture studies of tomato (Lycopersicon esculentum), Plant Cell Tiss. Org. Cult., 78(1) (2004), 1-21). Media improvement in these and like ways has been found to facilitate the growth of cells at specific developmental stages. Tissue may be maintained on a basic medium with growth regulators until sufficient tissue is available to begin plant regeneration efforts, or following repeated rounds of manual selection, until the morphology of the tissue is suitable for regeneration, then transferred to medium conducive to maturation of embryoids. Cultures are transferred as needed on this medium. Shoot development will signal the time to transfer to medium lacking growth regulators.

The transformed cells, identified by selection or screening and cultured in an appropriate medium that supports regeneration, will then be allowed to mature into plants. In some embodiments, callus tissue formation is induced from leaf disc. Callus tissue may be treated with hormone-supplemented medium to induce shoot elongation and root production. This tissue may then be transformed and grown into plantlets. Developing plantlets are transferred to soil. Plants may be matured in a growth chamber or greenhouse. Plants can be regenerated from about 6 wk to 10 months after a transformant is identified, depending on the initial tissue. During regeneration, cells are grown on solid medium in tissue culture vessels. Illustrative embodiments of such vessels are petri dishes and PlantCon™ containers. Regenerating plants can be grown at a suitable temperature, for instance about 19 to 28° C. After the regenerating plants have reached the stage of shoot and root development, they may be transferred to a greenhouse for further growth and testing. Tomato transformation methods are known in the art, see, for example, Sun et al. (Sun, H. J., Uchii, S., Watanabe, S. and Ezura, H. (2006) A highly efficient transformation protocol for Micro-Tom, a model cultivar for tomato functional genomics. Plant Cell Physiol. 47, 426-431).

To confirm the presence of the exogenous DNA or transgene(s) in the regenerating plants, a variety of assays may be performed. Such assays include, for example, molecular biological assays, such as Southern and northern blotting and PCR; biochemical assays, such as detecting the presence of a protein product, e.g., by immunological means (ELISAs and western blots) or by enzymatic function; plant part assays, such as leaf, seed, or root assays; and also, by analyzing the phenotype of the whole regenerated plant.

Very frequently, the expression of a gene product is determined by evaluating the phenotypic results of its expression. These assays also may take many forms including but not limited to analyzing changes in the chemical composition, morphology, or physiological properties of the plant. In some embodiments, engineered plants are exposed to stresses (e.g., extreme temperature, drought, slat solutions, etc.) alongside unmodified plants of the same genotype and stress response is evaluated relative to the control unmodified plant. Most often changes in response of plants or plant parts to imposed treatments are evaluated under carefully controlled conditions termed bioassays. Bioassays may evaluate and measure electrolyte leakage, decreases in photosystem II operating efficiency (Fy/Fm), necrosis, wilting, leaf rolling, decreased growth, and plant death.

Resistance to biotic stress is typically tested by exposing the engineered plants along with unmodified control plants to the pest or pathogen in question, and then measuring the survival and replication of the stressor, and/or measuring the plant symptoms induced by the stressor. Resistant plants would be expected to support lower pest populations and/or show less pronounced symptoms than control plants. Examples of symptoms that may be measured include defoliation, chlorosis, necrosis, galling, or reduced biomass. An example of an assay to measure pest survival and proliferation would be the aphid bioassays described in the Examples below.

Plant responses to abiotic stress are typically tested by exposing engineered plants along with unmodified control plants to a harsh environmental condition such as low temperature (typically ≤15° C. for tomato) or salinity (often reproduced by water with ≥50 mM sodium chloride solutions), and then measuring the plant symptoms induced by the stressor. Stress-tolerant plants would be expected to show less pronounced symptoms than control plants. Examples of symptoms that may be measured include electrolyte leakage from plant cells, decreases in photosystem II operating efficiency (Fᵥ/Fₘ), wilting, leaf malformation (crinkling, rolling, etc), necrosis, decreased growth, and even death of the plant.

### Decrease in Gene Expression

The present disclosure, in certain aspects, includes steps of reducing the function of a target gene to yield a desirable phenotype. In some embodiments, DNA may be introduced into the plant or plant cell to yield a non-natural modification to reduce expression of or silence a target gene selected from the group consisting of SlFAD2-1, SlFAD2-2, SlFAD6, or combinations to yield a phenotype with reduced linoleic acid and other polyunsaturated fatty acid content, increased oleic acid and other monounsaturated fatty acid content, and/or increased tolerance of stresses such as high temperature. A decrease in expression or function may be a decrease of at least 30%, at least 40%, at least 50%, at least 60%, at least 70%, at least 80%, at least 90%, or 100% when compared to a plant of the same genetic background without the non-natural modification. Decreased function or expression may be measured as a decrease in mRNA transcriptions (e.g., as measured by RT-PCR) or decreased protein production. A reduction in linoleic acid may be a reduction of at least 2%, at least 5%, at least 8%, at least 10%, at least 15%, at least 18%, at least 20%, or at least 25% when compared to a plant of the same genetic background without the non-natural modification. An increase in oleic acid may be an increase of at least 2%, at least 5%, at least 8%, at least 10%, at least 15%, at least 18%, at least 20%, or at least 25% when compared to a plant of the same genetic background without the non-natural modification. Linoleic acid and oleic acid may be measured in a specific tissue (e.g., seeds). An increase in heat tolerance may be an increase of at least 10%, at least 20%, at least 30%, at least 40%, or at least 50% when compared to a plant of the same genetic background without the non-natural modification.

Heat tolerance may be measured by comparing the health of modified plants and unmodified controls exposed to short-term heat stress (typically ≥38° C.) or long-term moderate heat conditions (typically ˜32° C.) at different growth stages. The appropriate measures of plant symptom development vary depending upon the growth stage, but can include plant water potential, stomatal conductance, photosy stem II operating efficiency (Fᵥ/Fₘ), electrolyte leakage from plant cells, wilting, leaf malformation (crinkling, rolling, etc), pollen production and viability, fruit set, necrosis, plant growth, and the incidence of premature plant death.

To that end, a polynucleotide may be introduced into plants for the purpose of expressing transcripts that function to affect plant phenotype. Example include, CRISPR/Cas mediated genome editing, RNA interference (RNAi), virus mediated gene silencing, and RNA with ribozyme activity. These may serve possible functions in reducing or eliminating expression of native or introduced plant genes. In some embodiments, RNA itself is introduced into the plant or plant cell. Transformation of polynucleotides into plant and tomato cells may be carried out as previously described herein.

Gene editing using the precise targeting process of Clustered, Regularly Interspaced Short Palindromic Repeats (CRISPR) combined with the Cas9 nuclease to make a double stranded break (collectively referred to as CRISPR/Cas9 or CRISPR/Cas9 system) may be used to reduce the function of or silence a target gene as described herein. The site of the break is targeted by short guide RNA (sgRNA or gRNA) often about 20 nucleotides in length. The break can be repaired by non-homologous end joining (NHEJ) or homology-directed recombination to make a desired mutation or modification at the cut site. Suitable CRISPR/Cas9 nucleases and vectors are known in the art. See for example US20180273961, U.S. Pat. No. 8,771,945, Reem et al. (Reem N. T., Van Eck J. (2019) Application of CRISPR/Cas9-Mediated Gene Editing in Tomato. In: Qi Y. (eds) Plant Genome Editing with CRISPR Systems. Methods in Molecular Biology, vol 1917. Humana Press, New York, N.Y.), and Brooks et al. (Brooks C., Nekrasov V., Lippman Z. B., Van Eck J. (2014). Efficient gene editing in tomato in the first generation using the clustered regularly interspaced short palindromic repeats/CRISPR-associated9 system. Plant Physiol. 166 1292-1297).

In another approach, it is possible that genes or nucleic acid sequences may be introduced into plants or plant cells to produce novel engineered plants which have reduced or silenced expression of a native gene product by the mechanism of RNAi. RNAi strategies, including double stranded RNA (dsRNA), hairpin RNA (hpRNA), and small interfering RNA (siRNA), have been shown to be effective in silencing gene expression in plants. RNAi inhibits gene expression in a sequence specific fashion, typically occurring in two steps: First, cleavage of longer dsRNA or hpRNA into shorter, 21- to 25-nucleotide-siRNA. In the second step, siRNA mediate degradation of a target mRNA molecule expressed from the target gene to be silenced. To achieve this effect either the dsRNA/hRNA or the siRNA can be introduced or transfected into plant cells or tomato cells. RNAi may also be achieved using microRNA (miRNA), which is small non-coding RNA complementary to the mRNA expressed form the target gene with functions to reduce or inhibit translation fo the mRNA.

Genes may be constructed or isolated, which when transcribed, produce miRNA or siRNA that is complementary to all or part(s) of a targeted messenger RNA(s). Genes may also be constructed or isolated, which when transcribed, produce dsRNA or hpRNA specific to the target genes described herein. Vectors or gene constructs designed to express miRNA, siRNA, dsRNA, and/or hpRNA may be introduced or transfected into plant cells or tomato cells to silence expression of one or more target genes described herein.

Gene expression may also be down regulated using virus induced gene silencing (VIRS). Suitable methods for VIRS are known and used in the art. See for example Liu et al. (Liu et al., “Virus-induced gene silencing in tomato,” Plant J., 2002, 31:777-786).

Genes also may be constructed or isolated, which when transcribed, produce RNA enzymes (ribozymes) which can act as endoribonucleases and catalyze the cleavage of RNA molecules with selected sequences. The cleavage of selected messenger RNAs can result in the reduced production of their encoded polypeptide products. These genes may be used to prepare novel transgenic plants that possess them. The transgenic plants may possess reduced levels of polypeptides including, but not limited to, the polypeptides corresponding to the target genes described herein.

Ribozymes are RNA-protein complexes that cleave nucleic acids in a site-specific fashion. Ribozymes have specific catalytic domains that possess endonuclease activity. For example, a large number of ribozymes accelerate phosphoester transfer reactions with a high degree of specificity, often cleaving only one of several phosphoesters in an oligonucleotide substrate. This specificity has been attributed to the requirement that the substrate bind via specific base-pairing interactions to the internal guide sequence (“IGS”) of the ribozyme prior to chemical reaction.

Several different ribozyme motifs have been described with RNA cleavage activity. Examples include sequences from the Group I self-splicing introns including Tobacco Ringspot Virus, Avocado Sunblotch Viroid, and Lucerne Transient Streak Virus. Sequences from these and related viruses are referred to as hammerhead ribozyme based on a predicted folded secondary structure.

Other suitable ribozymes include sequences from RNase P with RNA cleavage activity, hairpin ribozyme structures and Hepatitis Delta virus based ribozymes. The general design and optimization of ribozyme directed RNA cleavage activity is well understood in the art.

The other variable on ribozyme design is the selection of a cleavage site on a given target RNA. Ribozymes are targeted to a given sequence by virtue of annealing to a site by complimentary base pair interactions. Two stretches of homology are required for this targeting. These stretches of homologous sequences flank the catalytic ribozyme structure defined above. Each stretch of homologous sequence can vary in length from 7 to 15 nucleotides. The only requirement for defining the homologous sequences is that, on the target RNA, they are separated by a specific sequence that is the cleavage site. For hammerhead ribozyme, the cleavage site is a dinucleotide sequence on the target RNA is a uracil (U) followed by either an adenine, cytosine or uracil (A, C or U). The frequency of this dinucleotide occurring in any given RNA is statistically 3 out of 16. Therefore, for a given target messenger RNA of 1000 bases, 187 dinucleotide cleavage sites are statistically possible.

Designing and testing ribozymes for efficient cleavage of a target RNA is a process well known to those skilled in the art. The identification of operative and preferred sequences for use in down regulating a given gene is simply a matter of preparing and testing a given sequence, and is a routinely practiced “screening” method known to those of skill in the art.

The present invention has been described in terms of one or more preferred embodiments, and it should be appreciated that many equivalents, alternatives, variations, and modifications, aside from those expressly stated, are possible and within the scope of the invention.

### Example 1

This embodiment focuses on members of the FAD2 family, which includes Δ-12 desaturases and enzymes with divergent activities. Although this family occurs throughout the plant kingdom, knowledge of these enzymes is based largely on research on the model plant Arabidopsis (Arabidopsis thaliana) and on oil seed crops. The Arabidopsis FATTY ACID DESATURASE 2 (AtFAD2) gene was the first FAD2 gene to be described, and encodes a Δ-12 desaturase localized in the endoplasmic reticulum (ER) (Falcone et al., 1994; Okuley et al., 1994). Together with FATTY ACID DESATURASE 6 (FADE), a Δ-12 FAD localized in the chloroplast, the FAD2 enzyme enables the accumulation of polyunsaturated fatty acids in plants by adding a second double bond to the monounsaturated fatty acid oleic acid (18:1^{Δ9}) at the Δ-12 position in the acyl chain (Ohlrogge and Browse, 1995). This yields linoleic acid (18:2^{Δ9,12}), which can in turn be converted to linolenic acid (18:3^{Δ9,12,15}) by Δ-15 (i.e. ω-3) fatty acid desaturases (namely FAD3 in the ER and FAD7 and FAD8 in the chloroplast) (Ohlrogge and Browse, 1995). Linoleic acid and linolenic acid are among the most abundant polyunsaturated fatty acids in plants, and their accumulation is strongly influenced by levels of FAD2 gene expression and enzyme activity (Mei et al. 2015; Dar et al. 2017).

Homologs of the Arabidopsis FAD2 gene (AtFAD2) have been identified in a diverse array of oil seed crops because of commercial interest in modifying the fatty acid content of oil by manipulating FAD2 activity in seeds. Numerous studies have demonstrated that suppressing the Δ-12 desaturase activity of FAD2 isomers in seeds reduces the abundance of linoleic and linolenic acid relative to oleic acid in the storage lipids of the seed (Dar et al., 2017). This decrease in polyunsaturation is considered a desirable trait in oil seed crops because it enhances the oxidative stability and shelf life of the oil, reducing rancidity and improving the industrial properties of the oil (Clemente and Cahoon, 2009). Because of this, the oil seed quality of canola (Brassica napus [Schierholt et al., 2000]), peanut (Arachis hypogae [Jung et al. 2000]), cotton (Gossypium hirsutum [Chapman et al., 2001]), soybean (Glycine max [Buhr et al., 2002]), sunflower (Helianthus annuus, [Schuppert et al., 2006]), safflower (Carthamus tinctorius [Hamdan et al. 2012]), and other oil seed crops has been enhanced by decreasing endoplasmic Δ-12 desaturase activity through means such as genome editing (Haun et al. 2014), chemical mutagenesis (Lee et al. 2018), RNA interference (Buhr et al. 2002), and selection for naturally-occurring allelic variation in FAD2 genes (Thambugala et al., 2013), and several of these high-oleic acid crops are commercially widespread.

Despite the predominant research focus on seed-localized FAD2 isomers with known Δ-12 desaturase activity, other studies indicate that the FAD2 family also contains considerable diversity in function and expression patterns. This diversification is enabled by the presence of multigene families; whereas the Arabidopsis genome carries only one copy of AtFAD2, comparative analysis of other species reveals that many plant genomes carry multiple FAD2 homologs. For example, soybean has seven FAD2 family members (Lakhssassi et al., 2017), cotton has nine (Feng et al. 2017), safflower has eleven (Cao et al. 2013), and parsley and carrot have the highest number of family members documented so far (17 and 24, respectively), indicating that functional divergence of FAD2 is not limited to oil seed crops (Somssich et al. 1989; Busta et al. 2018). Although the functions of the majority of FAD2 homologs are untested, analyses of plant species that produce unusual fatty acids or fatty acid derivatives have identified so-called divergent FAD2 proteins with novel enzymatic capabilities. FAD2 family members that encode hydroxylases (Broun et al., 1998a; Van De Loo et al., 1995), epoxidases (Lee et al., 1998), acetylenases (Cahoon et al., 2001; Okada et al., 2013; Busta et al., 2018), or conjugases (Cahoon et al., 1999; Liu et al., 2001; Dyer et al., 2002; Cahoon and Kinney, 2004) have been identified in a taxonomically diverse array of medicinal species and other plants with unusual seed chemistries, including the castor oil plant (Ricinus communis, Euphorbiaceae), popweed (Lesquerella fendleri, Brassicaceae), hawksbeard (Crepis spp., Asteraceae), sweet quandong (Santalum acuminatum, Santalaceae) bitter melon (Momordica charantia, Cucurbitaceae), touch-me-not (Impatiens balsamina, Balsaminaceae) and carrot (Daucus carota, Apiaceae). Despite the evidence for functional diversification of FAD2 in multiple plant families, the potential for such diversification in major crops has been largely unexplored.

In addition to encoding catalytic diversity, FAD2 homologs also show diversity in their expression patterns. While some FAD2 genes are targeted to the seeds, others are ubiquitous or specific to other organs; for example, transcript profiling of the FAD2 gene family in cotton and safflower revealed that developing seeds, roots, stems, and leaves expressed overlapping but distinct sets of FAD2 homologs (Cao et al., 2013; Feng et al. 2017). In addition, different members of the FAD2 family in soybean varied in their responsiveness to chilling and salinity (Feng et al., 2017), two abiotic stresses known to upregulate FAD2 expression (Wang et al., 2004; Kargiotidou et al., 2008; Teixeira et al., 2009; Zhang et al., 2009). In other species, some FAD2 genes are upregulated by biotic stresses, including viroid infection (Gadea et al., 1996), fungal infection (Wang et al. 2004), or application of fungal elicitors (Kirsch et al., 1997; Cahoon et al. 2003). FAD2 family members with desaturase activity are thought to contribute to adaptation to abiotic stress by altering the properties of cell membranes (Zhang et al. 2012), and divergent FAD2 genes that encode acetylenases are reported to synthesize secondary metabolites with antibiotic properties (Cahoon et al. 2003). Together, the diversity of enzymatic capabilities and expression patterns within the FAD2 family suggest a diversity of functions that have not yet thoroughly been explored, including possible roles in biotic stress resistance.

The embodiment described herein characterizes the FAD2 family in tomato, Solanum lycopersicum. This work expands the understanding of FAD2 family members to a major crop other than oil seed producers, and examines their stress-responsive expression patterns and functions in vegetative tissues. Genomic analysis identified nine FAD2 genes in tomato. Of these nine genes, only two had Δ-12 desaturase activity when ectopically expressed in yeast, indicating that SlFAD2-1 and SlFAD2-2 are the family members responsible for linoleic acid synthesis in tomato. Pathway analysis and RT-qPCR further indicated that the nine genes had distinctly different expression patterns, and that some family members are stress-responsive in foliage. Whereas SlFAD2-1 and SlFAD2-2 were unresponsive to mechanical wounding or aphids (Macrosiphum euphorbiae) and were downregulated by a bacterial pathogen (Pseudomonas syringae pv. DC3000), five out of seven of the other family members were transcriptionally upregulated by one or more of these stresses. SlFAD2-4 and SlFAD2-7 showed particularly strong and broad stress responses; they were induced in foliage by wounding, aphid infestation, bacterial infection, and exogenous application of salicylic acid, a key signaling molecule in biotic stress. Levels of constitutive foliar expression of SlFAD2-4 and SlFAD2-7 were also enhanced by spr2, a mutation in FATTY ACID DESATURASE 7 (LeFAD7) that increases accumulation of linoleic acid and hexadecadienoic acid (16:2^{Δ9,12}) and promotes aphid resistance. Furthermore, virus induced gene silencing (VIGS) of SlFAD2-4 or SlFAD2-7 in the spr2 mutant compromised aphid resistance in this genotype, resulting in increased aphid population growth on silenced plants. These results indicate that SlFAD2-4 and SlFAD2-7 can promote plant defenses against aphids in certain genetic backgrounds. In summary, the FAD2 gene family in tomato includes multiple divergent members in addition to two standard Δ-12 desaturases, and that divergent family members influence plant interactions with aphids and other biotic stresses.

### Results

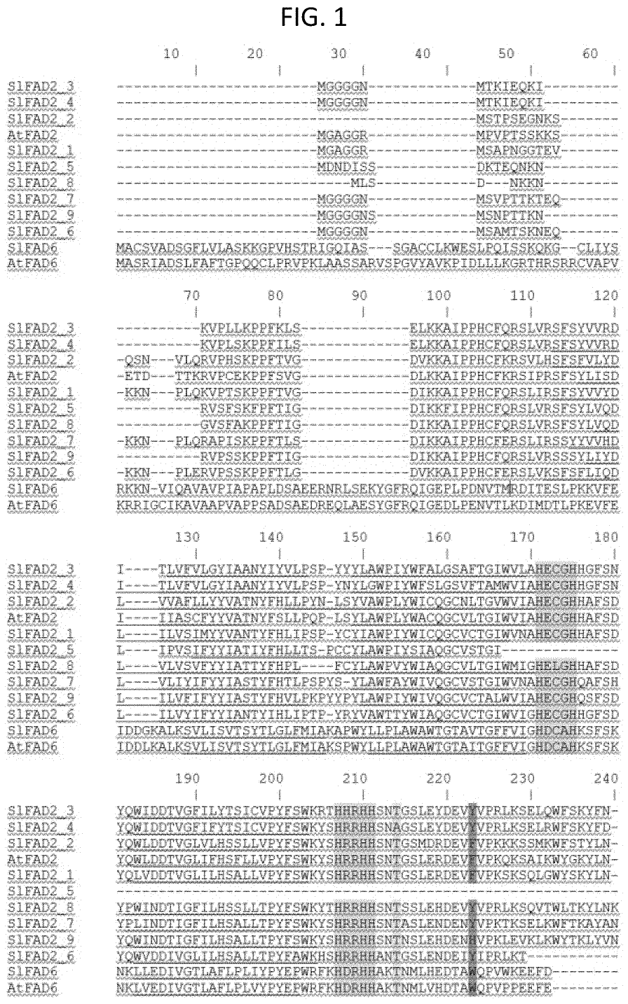

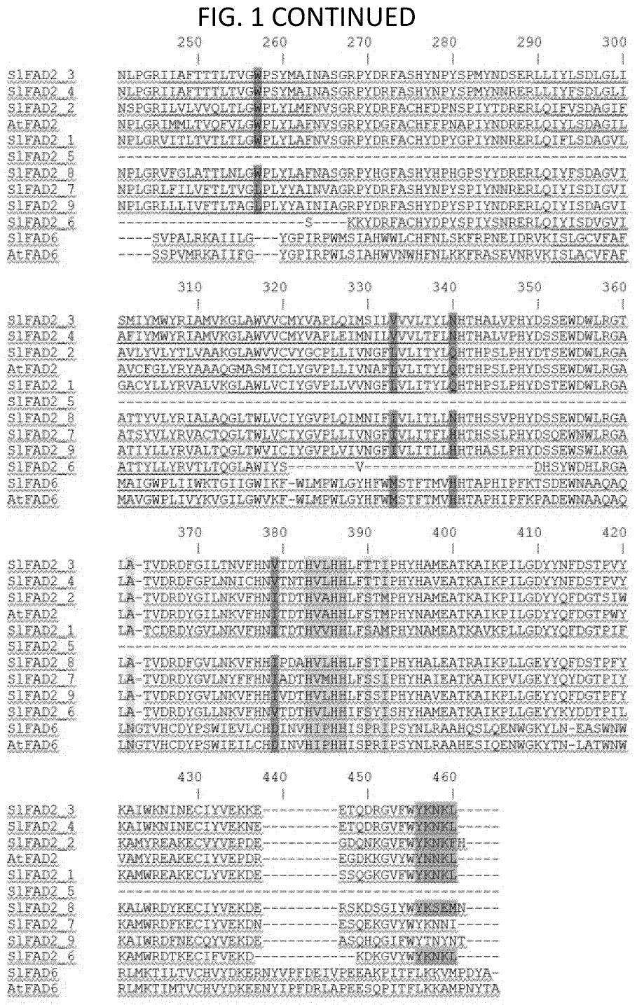

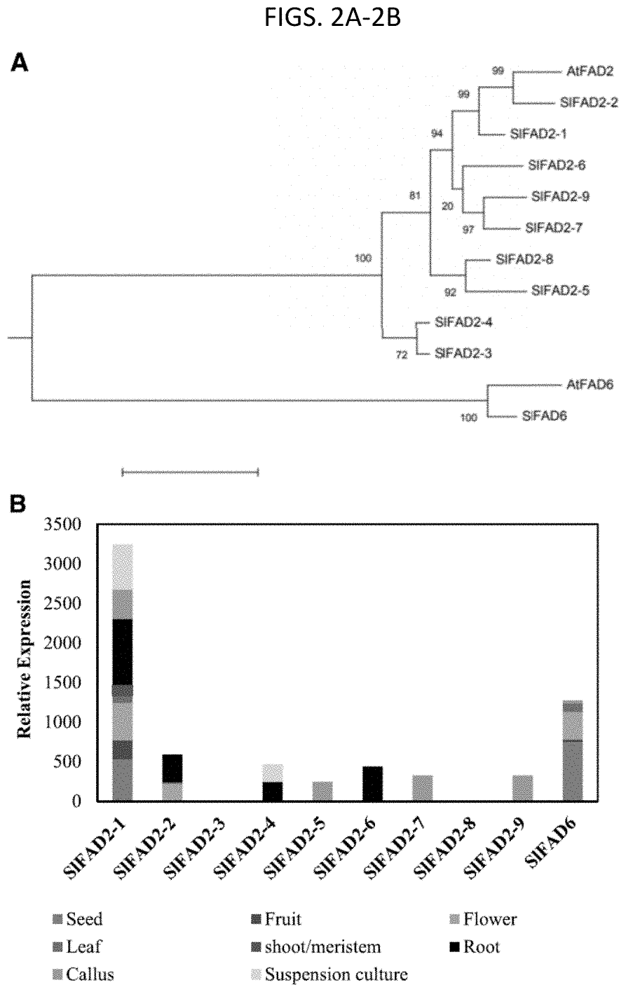

The tomato genome contains nine homologs of the Arabidopsis AtFAD2 gene and one homolog of AtFAD6—A search of the tomato genome (ITAG release 2.40) for homologs of the Arabidopsis AtFAD2 gene identified nine homologs, which are located on Chromosomes 1 (SlFAD2-1), 3 (SlFAD2-2), 4 (SlFAD2-3 and SlFAD2-4), and 12 (SlFAD2-5, SlFAD2-6, SlFAD2-7, SlFAD2-8 and SlFAD2-9). Based on an alignment of their predicted amino acid (AA) sequences (FIG. 1), their percent shared identities with AtFAD2 range from 52-76% (Table 2). For comparison, we also searched for homologs of the Arabidopsis chloroplastic Δ12-desaturase AtFAD6. In contrast to the SlFAD2 gene family, the FADE enzyme in tomato appears to be encoded by a single gene, SlFAD6, which is located on Chromosome 7 and shares about 81.56% sequence identity with AtFAD6. A phylogenetic analysis of the deduced polypeptide sequences for these 10 tomato genes and for AtFAD2 and AtFAD6 revealed that these sequences separate into five distinct groups (FIG. 2A). As expected, SlFAD6 clusters with AtFAD6 and forms a unique group separate from all FAD2 sequences. Of the SlFAD2 family members, SlFAD2-1 and SlFAD2-2 are the most closely aligned to AtFAD2, and form a discrete cluster with AtFAD2 (FIG. 2A).

TABLE 2
Gene identities and primer sequences for cloning and gene expression analysis.

| | SOL Genomics | % AA | E | Primers for Cloning | Size³ |
|---|---|---|---|---|---|
| Gene | Sequence | Identitv¹ | value² | (5′Foiward 3′; 5′Reverse 3′) | (bP) |
| SlFAD2 1 | Solyc01g006430 | 73.89 | ~0.0⁴ | F:CACCATGGGAGCTGGTGGTCGTAT (SEQ ID NO: 1) | 1152 |
| | | | | R:TCAGAGCTTGTTTTTGTACC (SEQ ID NO: 2) | |
| SlFAD2-2 | Solyc03g058430 | 75.86 | ~0.0 | F:CACCATGTCCACTCCTTCTGAGGGC (SEQ ID NO: 3) | 1137 |
| | | | | R:CTAATGAAACTTGTTTTTATACC (SEQ ID NO: 4) | |
| SlFAD2-3 | Solyc04g040120 | 58.49 | e-173 | F:CACCATGGGAGGTGGTGGTAATATGA (SEQ ID NO: 5) | 1128 |
| | | | | R:TTAGAGTTTGTTTTTATACCAAA (SEQ ID NO: 6) | |
| SlFAD2-4 | Solyc04g040130 | 57.96 | e-172 | F:CACCATGGGAGGTGGTGGTAATATGA (SEQ ID NO: 7) | 1128 |
| | | | | R:TTAGAGTTTGTTTTTATACCA (SEQ ID NO: 8) | |
| SlFAD2-5 | 5o1ye12g036520 | 59.55 | 5e-28 | F:CACCATGGACAACGATATATCATCCGA (SEQ ID NO: 9) | 282 |
| | | | | R:CTATATTCCAGTAGAAACACAA (SEQ ID NO: 10) | |
| SlFAD2-6 | Solyc12g044950 | 52.48 | e-141 | F: CACCATGGGAGGTGGTGGTAATATGTC (SEQ ID NO: 11) | 951 |
| | | | | R:TCAAAGCTTGTTTTTGTACCAA (SEQ ID NO: 12) | |
| SlFAD2-7 | Solyc12g049030 | 63.80 | ~0.0 | F:CACCATGGGAGGTGGTGGTAATATGTC (SEQ ID NO: 13) | 1155 |
| | | | | R:TCAAATATTGTTTTTGTACCA (SEQ ID NO: 14) | |
| SlFAD2-8 | Solycl2g100230 | 65.49 | ~0.0 | F:CACCATGTTGTCTGATAACAAGAAAAA (SEQ ID NO: 15) | 1107 |
| | | | | R:TTAATTCATCTCACTTTTATAC (SEQ ID NO: 16) | |
| SlFAD2-9 | Solycl2g100250 | 63.71 | ~0.0 | F:CACCATGGGAGGTGGTGGTAATTCTAT (SEQ ID NO: 17) | 1140 |
| | | | | R:TTATGTGTTGTAATTTGTATAC (SEQ ID NO: 18) | |
| SlFAD6 | Solyc07g005510 | 82 | ~0.0 | F:CACCATGGCTTGCAGTGTTGCAGACTCT (SEQ ID NO: 19) | 1326 |
| | | | | R:TCAAGCGTAGTCAGGCATTACT (SEQ ID NO: 20) | |

| Gene | Primers for RT- qPCR | Size³ (bp) |
|---|---|---|
| SlFAD2 1 | F:TCCTTGTCCTGATCACCTAC (SEQ ID NO: 21) | 323 |
| | R:GAGATGATTCGTCTTTCTCG (SEQ ID NO :22) | |
| SlFAD2-2 | F:GTTGTCCAGCTCACTCTAGG (SEQ ID NO: 23) | 310 |
| | R:TGTCATAATGAGGCAATGAA (SEQ ID NO: 24) | |
| SlFAD2-3 | F:TGGAAACGTACTCATCATCGTC (SEQ ID NO: 25) | 509 |
| | R:TGAAAAACGTTAGTTAGTATACCAA (SEQ ID NO: 26) | |
| SlFAD2-4 | F:GAAGGCTATTCCTCCTCATT (SEQ ID NO: 27) | 319 |
| | R:ACGACGATGACTGTATTTCC (SEQ ID NO: 28) | |
| SlFAD2-5 | F:TGGACAACGATATATCATCC (SEQ ID NO: 29) | 280 |
| | R:ACCTTGAGCAATCGAGTAAA (SEQ ID NO: 30) | |
| SlFAD2-6 | F:CTTCAAAGCCTCCTTTTACA (SEQ ID NO: 31) | 323 |
| | R:GTGGAGGATAAGACCAACAA (SEQ ID NO: 32) | |
| SlFAD2-7 | F:ACTGCTTCCCTTGAGAACGA (SEQ ID NO: 33) | 441 |
| | R:GCTCCTCGTAGCCAATTCCA (SEQ ID NO: 34) | |
| SlFAD2-8 | F:ATGGCTTTGCAAGTCACTAT (SEQ ID NO: 35) | 325 |
| | R:GAATGTGGTGGAAGACCTTA (SEQ ID NO: 36) | |
| SlFAD2-9 | F:ACCCCTTGTAATTGTGAATG (SEQ ID NO: 37) | 355 |
| | R:AGATGCTTCATCTTTCTCCA (SEQ ID NO: 38) | |
| SlFAD6 | F:TGTTGGAACACTAGCCTTTT (SEQ ID NO: 39) | 438 |
| | R:TCCAAAAATGATAACCCAAC (SEQ ID NO: 40) | |

¹Identity in common with the nearest homolog in Arabidopsis, which is AtFAD2 (At3g12120) for all SIFAD2 family members, and AtFAD6 (At4g30950) for SlFAD6.
²Statistical significance of homology to closest Arabidopsis homolog according to BLASp (AtFAD6 for SlFAD6; AtFAD2 for all others)
³Amplicon size
⁴E values smaller than ~5 * 10⁻³²⁴ rounded to zero

Members of the SlFAD2 family share transmembrane domains and common motifs associated with iron-binding and ER localization—Regardless of their specificity, almost all membrane-bound desaturases in plants contain three histidine boxes, which are hypothesized to serve as ligands for a diiron cluster at the active site of the enzyme (Shanklin and Cahoon, 1998). Consistent with this, a tripartite motif of conserved histidines was identified in the predicted AA sequences of SlFAD6 and all members of the SlFAD2 family with the exception of SlFAD2-5 (FIG. 1). As expected for membrane-associated desaturases, all of the predicted amino acid sequences also contained between four and six predicted transmembrane domains with the exception of the SlFAD2-5 sequence, which only contained one predicted transmembrane domain. The predicted SlFAD2-5 protein is highly truncated compared to other family members, lacking all three histidine boxes and nine amino acid positions that were previously described to be important to enzyme activity (FIG. 1) (Broun et al. 1998; Gagne et al. 2009); therefore, it may potentially be a pseudogene. In all other genes in our analysis, the locations of the three histidine boxes align, and their predicted AA sequences distinguish FAD2 homologs from members of the FADE family. For AtFAD2 and all eight full-length SlFAD2 genes, the consensus sequences for the three histidine boxes are HEXGH, HXRHH, and HVXHH respectively (FIG. 1). In contrast, the first and third histidine boxes in in SlFAD6 and AtFAD6 are HDCAH and HIPHH, which match motifs (HDCXH and HXPHH) previously reported to be characteristic of FAD6 genes in multiple plant species (Chi et al. 2011). Most of the full-length SlFAD2 genes are also distinguished from SlFAD6 by the presence of an ER retrieval motif at the C terminus (positions 456-460 in FIG. 1). SlFAD2-1, SlFAD2-2, SlFAD2-3, SlFAD2-4, SlFAD2-6, and SlFAD2-8 all contain a conserved sequence (Y-X-X-K/R/D/E-Φ, where Φ represents a large hydrophobic amino acid such as L, F, M, or I) that is absent in SlFAD6, and that has previously been shown to be necessary and sufficient to localize the FAD2 protein in the ER (McCartney et al. 2004). These results support the categorization of SlFAD2 genes as members of the FAD2 family.

Members of the SlFAD2 family display sequence divergence at sites associated with enzyme function—Previously, targeted mutagenesis identified four AA positions that, when modified, could convert the AtFAD2 Δ-12 desaturase into a hydroxylase (Broun et al. 1998b; Broadwater et al. 2002). A comparative analysis of FAD2 Δ-12 desaturases and FAD2 acetylenases from multiple plant species also identified five AA positions that consistently differed between these two groups of enzymes, and that, when mutagenized, modified the chemoselectivity, stereoselectivity, and substrate recognition of desaturases and acetylenases (Gagne et al. 2009). Therefore, to investigate the potential functions of members of the SlFAD2 family, we compared their AA sequences with respect to these nine positions that are known to regulate enzymatic activity. SlFAD2-1 and SlFAD2-2 are the only members of the SlFAD2 family that are identical to AtFAD2 at all nine of these critical residues (FIG. 1), and their predicted AA sequences differed from all of the other SlFAD2 proteins at four of these sites (positions 223, 333, 340, and 392 in FIG. 1). Conversely, SlFAD4 is the most divergent of the full-length members of the SlFAD2 family, and differs from AtFAD2 at seven out of nine of these residues, including two AA positions reported by Broadwater and coworkers (2002) to be particularly important to catalytic specificity (positions 214 and 392 in our alignment, FIG. 1). Although most members of the SlFAD2 family diverged from the expected AA sequence for a Δ-12 desaturase, they also diverged from the sequences reported for known hydroxylases and acetylenases at these nine critical AA positions (Broun et al. 1998b; Broadwater et al. 2002; Gagne et al. 2009), matching at most one out of four positions that confer hydroxylase activity and two out of five positions important to acetylenase activity. This sequence analysis suggests that SlFAD2-1 and SlFAD2-2 are the most likely candidates for Δ12-desaturases in tomato, while SlFAD2-3 through SlFAD2-9 may have alternative functions.

Members of the SlFAD2 family differ in their tissue expression patterns—To explore when and where members of the SlFAD2 family are expressed, digital expression analysis of tomato ESTs (TIGR tomato gene index release 9.0) was performed to assess expression profiles of this family in various tissues including seed, fruit, flower, leaf, shoot, root, callus and suspension culture. Transcripts were detected for all genes except SlFAD2-3 and SlFAD2-8, and varied greatly in the tissues in which they were detected (FIG. 2B). SlFAD2-1 and SlFAD6 were the most abundant and ubiquitous transcripts, and showed strong expression levels in seeds, a primary site of fatty acid synthesis.

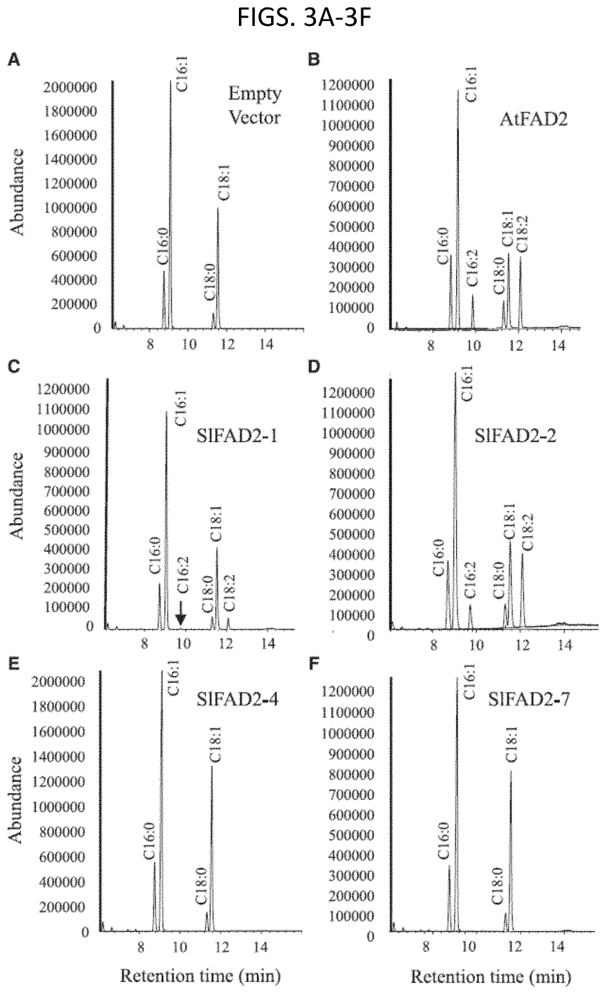

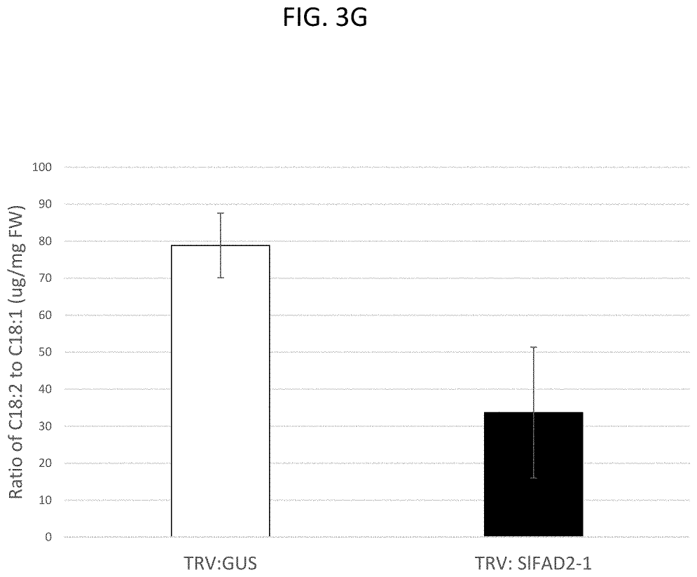

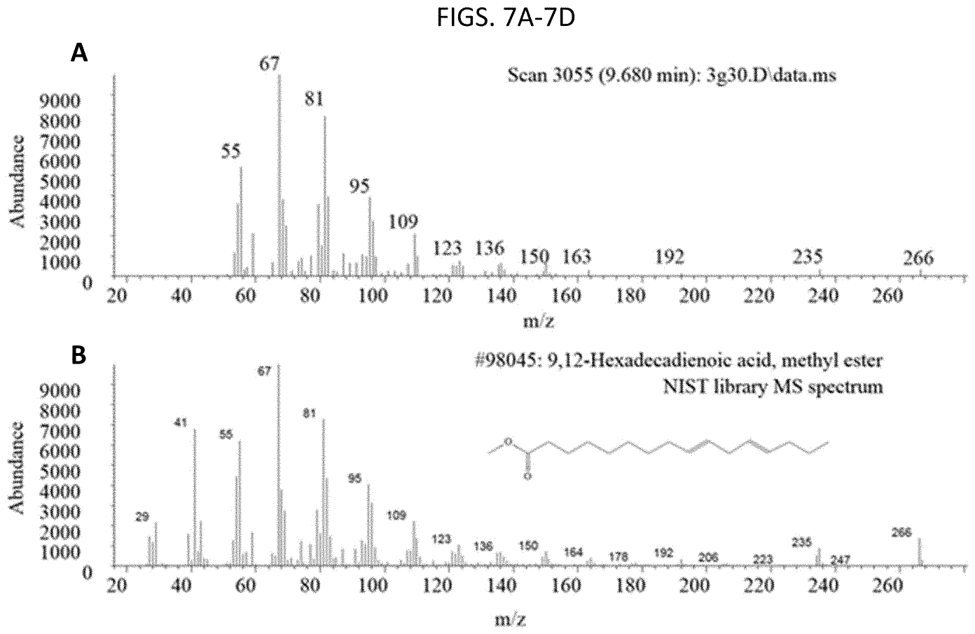

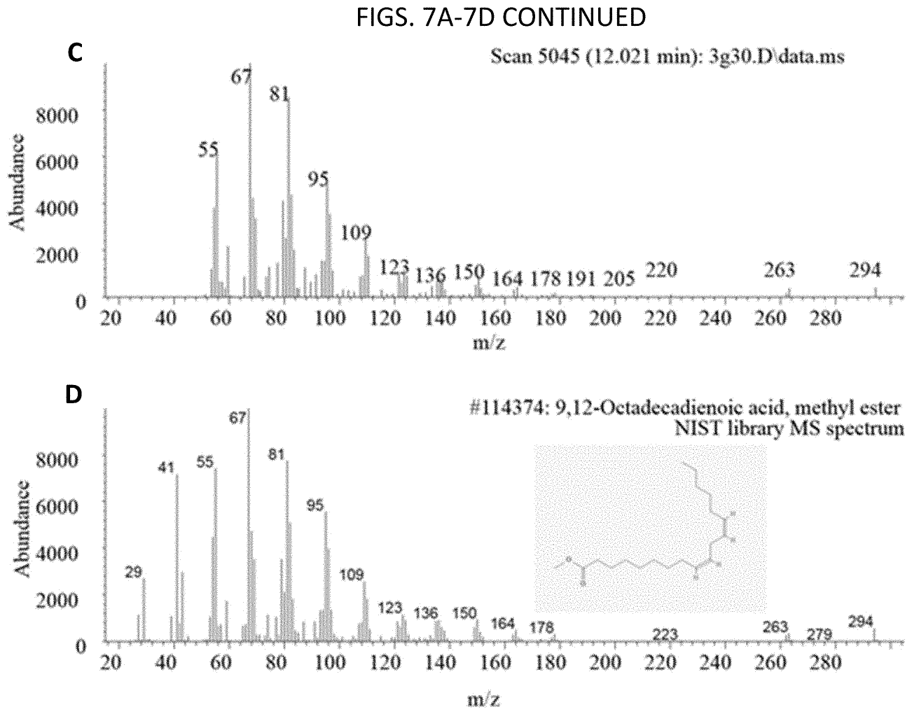

SlFAD2-1 and SlFAD2-2, but not other family members, confer/112-desaturase activity when expressed in yeast—To determine whether any of the SlFAD2 genes encode functional Δ12-desaturases, these genes were expressed in Saccharomyces cerevisiae under the control of the galactose-inducible GALACTOKINASE 1 (GAL 1) promoter, and fatty acid methyl ester (FAMES) profiles of the yeast cultures were analyzed by gas chromatography-mass spectrometry (GC-MS) to detect the presence of the Δ12-desaturase products, C16:2^{Δ9,12} and C18:2^{Δ9,12} when cultures were grown in the presence of galactose. S. cerevisiae is a suitable system in which to test for 412-desaturase activity of transgenes because this yeast lacks native Δ12-desaturases, but accumulates the necessary C16:1^{Δ9} and C18:1^{Δ9} substrates for these enzymes (Kailwara et al. 1996). Yeast cells transformed with the vector expressing the β-glucuronidase enzyme (GUS) served as a vector control, and lacked any peaks corresponding to C16:2^{Δ9,12} or C18:2^{Δ9,12} (FIG. 3A). In contrast, yeast cells transformed with the FAD2 gene from A. thaliana (AtFAD2) served as a positive control, and accumulated C16:2^{Δ9,12} and C18:2^{Δ9,12} (FIG. 3B). Of the nine FAD2 homologs from tomato, SlFAD2-1 and SlFAD2-2 yielded C16:2^{Δ9,12} and C18:2^{Δ9, 12} when expressed in yeast (FIGS. 3C-3D), whereas all other family members lacked detectable Δ12-desaturase activity (FIGS. 3E-3F and Table 3). The identities of C16:2^{Δ9,12} and C18:2^{Δ9,12} in all samples were confirmed by comparing the retention times of their GC peaks to those of purified standards, and also through analysis of their mass spectra (FIGS. 7A-7D). In summary, these results indicated that 412-desaturase activity was limited to SlFAD2-1 and SlFAD2-2. This is consistent with our prior observations that, within the SlFAD2 gene family, SlFAD2-1 and SlFAD2-2 had the highest homology to the Arabidopsis AtFAD2 gene (FIG. 2A), and the highest degree of conservation at AA positions that have previously been identified as important to enzyme activity (FIG. 1).

TABLE 3
Fatty acid composition of yeast cells expressing SlFAD2 genes

| Tranformants | C16:0 | C16:1 | C16:2 | C18:0 | C18:1 | C18:2 |
|---|---|---|---|---|---|---|
| GUS | 12.5 ± 0.13 | 53.6 ± 0.65 | 0 ± 0 | 3.5 ± 0.28 | 26.3 ± 1.60 | 0 ± 0 |
| SlFAD2-1 | 16.5 ± 0.20 | 48.3 ± 0.62 | 0.7 ± 0.01 | 4.4 ± 0.04 | 23.7 ± 0.28 | 6.1 ± 0.05 |
| SlFAD2-2 | 12.7 ± 0.25 | 46.4 ± 0.83 | 4.1 ± 0.07 | 4.5 ± 0.08 | 16.6 ± 0.07 | 12.8 ± 0.08 |
| SlFAD2-3 | 10.9 ± 0.64 | 50.0 ± 2.87 | 0 ± 0 | 3.3 ± 0.11 | 27.1 ± 1.20 | 0 ± 0 |
| SlFAD2-4 | 13.1 ± 0.14 | 50.8 ± 1.25 | 0 ± 0 | 3.7 ± 0.20 | 31.3 ± 1.29 | 0 ± 0 |
| SlFAD2-5 | 10.3 ± 0.06 | 51.9 ± 0.37 | 0 ± 0 | 4.2 ± 0.04 | 30.5 ± 0.25 | 0 ± 0 |
| SlFAD2-6 | 9.8 ± 0.02 | 50.7 ± 1.27 | 0 ± 0 | 3.3 ± 0.33 | 30.0 ± 0.33 | 0 ± 0 |
| SlFAD2-7 | 12.5 ± 0.06 | 49.9 ± 0.37 | 0 ± 0 | 3.3 ± 0.37 | 31.1 ± 0.97 | 0 ± 0 |
| SlFAD2-8 | 12.6 ± 0.01 | 54.8 ± 0.07 | 0 ± 0 | 3.7 ± 0.01 | 25.5 ± 0.03 | 0 ± 0 |
| SlFAD2-9 | 12.7 ± 0.22 | 53.5 ± 0.93 | 0 ± 0 | 3.5 ± 0.04 | 25.9 ± 0.40 | 0 ± 0 |
| AtFAD2 | 12.2 ± 0.10 | 33.9 ± 0.02 | 11.2 ± 0.06 | 5.4 ± 0.09 | 14.0 ± 0.15 | 21.8 ± 0.66 |

Virus Induced Gene Silencing of SlFAD2-1 decreases C18:2 accumulation in tomato foliage. Since SlFAD2-1 had Δ12-desaturase activity when it was ectopically expressed in yeast, we then utilized virus-induced gene silencing (VIGS) to test whether transiently suppressing SlFAD2-1 expression in tomato would inhibit C18:2 synthesis from C18:1. Tobacco Rattle Virus (TRV) vectors were used to silence SlFAD2-1 in the foliage of the spr2 mutant. This tomato genotype was chosen for testing because it accumulates higher-than-normal C18:2 levels. Foliar fatty acid profiles were compared in plants treated with the TRV:SlFAD2-1 silencing construct and plants infiltrated with vector controls (TRV:GUS). The ratio of C18:2 to its C18:1 precursor decreased by nearly 60% in foliage that received the TRV:SlFAD2-1 construct as opposed to the TRV:GUS vector control (FIG. 3G; p=0.0016). This provides evidence that SlFAD2-1 is involved in conversion of C18:1 to C18:2 in planta.

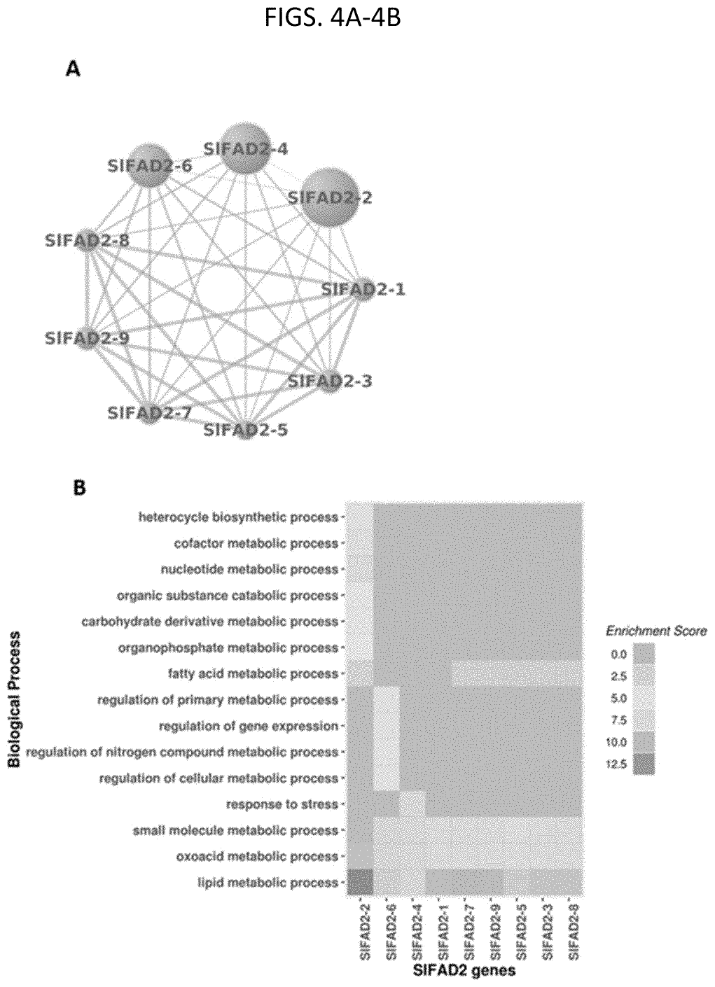

Members of the SlFAD2 family have overlapping but distinct protein interaction networks—To gain further insights into the putative functions of SlFAD2 genes, including those that do not appear to encode Δ12-desaturases, we searched the tomato Protein-Protein Interaction (PPI) network in the STRING database and extracted all putative interaction partners linked with each SlFAD2 gene. This database represents functional relatedness among genes derived by integrating information from multiple functional genomics data across many organisms (Szklarczyk et al. 2017). In the tomato PPI network, SlFAD2 genes are connected to 172 other genes on average (range=157-204) with extensive overlap among the interaction profiles of all SlFAD2 genes (FIG. 4A). As expected, the interaction profiles of all family members are significantly enriched in Gene Ontology (GO) Biological Process (BP) terms associated with lipid, oxoacid and small molecule metabolism (FIG. 4B). Compared to other family members, SlFAD2-2 is connected to the largest number of other genes (204) and the highest number of BP terms, suggesting that the FAD2-2 protein may be particularly important in primary metabolism. Interestingly, the BP term ‘response to stress’ is distinctly over-represented amongst genes that connect with SlFAD2-4, the family member with the second highest number of interaction partners (connected to 195 genes). These results suggest that SlFAD2-4 may be important in plant stress responses.

In silico promoter analysis identifies multiple hormone- and stress-responsive cis-regulatory elements for SlFAD2 family members—To investigate the putative regulatory mechanisms of the SlFAD2 gene family, we scanned the upstream promoter regions of the 9 SlFAD2 genes and identified 32 known plant cis-regulatory elements (CREs) that are over- or under-represented in comparison to the tomato genomic background (Table 4). Four out of nine members the SlFAD2 family (SlFAD2-1, SlFAD2-4, SlFAD2-5, and SlFAD2-8) are significantly over-represented with CREs associated with responsiveness to abscisic acid (ABA), a key hormone in plant development and environmental stress responses. Other hormone response elements that were over-represented included CREs for auxin (in SlFAD2-7 and SlFAD2-9) and gibberellins (GA, in SlFAD2-2 and SlFAD2-3). A majority of the SlFAD2 family was also enriched in stress-responsive regulatory elements, including CREs associated with drought (SlFAD2-1, SlFAD2-4, SlFAD2-5, SlFAD2-7, and SlFAD2-8), UV irradiation (SlFAD2-7), and fungal elicitors (SlFAD2-9). In particular, SlFAD2-4, SlFAD2-5, SlFAD2-7, and SlFAD2-8 all contain CREs with homology to the MYCATERD1 motif, which was first identified as a MYC recognition motif involved in transcriptional responses to water stress in Arabidopsis (Simpson et al. 2003). These results suggest that ABA, GA, and auxin play roles in regulating the SlFAD2 family, and that multiple members of this family may be stress-responsive.

TABLE 4
Cis-elements that are over- or underrepresented in the 2000 bp upstream promoters of SlFAD2 genes

| | | | | | | | | | | Asso- |
|---|---|---|---|---|---|---|---|---|---|---|
| | | | | | | | | | | ciation |
| | | | Expected | | | Signi- | Over/ | Best match in PLACE | Association | with |
| Tag | Motif | Occurance | Occ. | Pvalue | Evalue | ficance | Under | database | with Hormone | Stress |
| FAD2-1 | aaata | 0 | 10.61 | 2.40E-05 | 0.05 | 1.3 | under | TATABOX5 | | |
| M1 | a | | | | | | | | | |
| FAD2-1 | ctcgg | 4 | 0.18 | 3.40E-05 | 0.07 | 1.15 | over | DRE1COREZMRAB17 | abscisic | drought |
| M2 | a | | | | | | | | acid | |
| FAD2-1 | agacc | 5 | 0.47 | 0.00013 | 0.28 | 0.56 | over | QELEMENTZMZM13 | | |
| M3 | t | | | | | | | | | |
| FAD2-1 | taaaa | 1 | 10.74 | 0.00025 | 0.52 | 0.29 | under | SEF4MOTIFGM7S | | |
| M4 | a | | | | | | | | | |
| FAD2-1 | aaggt | 5 | 0.18 | 1.40E-06 | 0.01 | 1.93 | over | MSACRCYM | | |
| M5 | ct | | | | | | | | | |
| FAD2-1 | agtga | 4 | 0.13 | 1.20E-05 | 0.10 | 1.01 | over | SORLIP5AT | | |
| M6 | gg | | | | | | | | | |
| FAD2-2 | acact | 6 | 0.7 | 0.000087 | 0.18 | 0.74 | over | SORLIP5AT | | |
| M1 | c | | | | | | | | | |
| FAD2-2 | aaggc | 4 | 0.29 | 0.00025 | 0.52 | 0.29 | over | minus284M0TIFZMSBE1 | | |
| M2 | c | | | | | | | | | |
| FAD2-2 | atagtc | 6 | 0.85 | 0.00025 | 0.52 | 0.28 | over | SP8BFIBSP8BIB | | |
| M3 | | | | | | | | | | |
| FAD2-2 | aaaatt | 1 | 10.72 | 0.00025 | 0.52 | 0.28 | under | HSRENTHSR203J | | hyper- |
| M4 | | | | | | | | | | sensitive |
| | | | | | | | | | | response |
| FAD2-2 | aacaa | 8 | 1.67 | 0.00034 | 0.71 | 0.15 | over | GAREAT | gibberellins | |
| M5 | g | | | | | | | | | |
| FAD2-2 | aattat | 0 | 7.63 | 0.00048 | 1.00 | 0 | under | ATHB6COREAT | abscisic | |
| M6 | | | | | | | | | acid | |
| FAD2-3 | gtggt | 6 | 0.51 | 0.000016 | 0.03 | 1.48 | over | S1FBOXSORPS1L21 | | |
| M1 | a | | | | | | | | | |
| FAD2-3 | aaaaa | 2 | 13.62 | 0.00013 | 0.26 | 0.58 | under | SEF4MOTIFGM7S | | |
| M2 | t | | | | | | | | | |
| FAD2-3 | accac | 6 | 0.76 | 0.00014 | 0.29 | 0.54 | over | 5256BOXLELAT5256 | | |
| M3 | a | | | | | | | | | |
| FAD2-3 | gcttgt | 3 | 0.05 | 1.80E-05 | 0.58 | 0.24 | over | GAREAT | gibberellins | |
| M4 | aa | | | | | | | | | |
| FAD2-3 | accac | 3 | 0.05 | 2.10E-05 | 0.69 | 0.16 | over | POLLEN2LELAT52 | | |
| M5 | cac | | | | | | | | | |
| FAD2-4 | gtggt | 6 | 0.51 | 1.60E-05 | 0.03 | 1.48 | over | S1FBOXSORPS1L21 | | |
| M1 | a | | | | | | | | | |
| FAD2-4 | atgtg | 6 | 0.83 | 0.00022 | 0.46 | 0.33 | over | MYCATERD1 | | drought |
| M2 | g | | | | | | | | | |
| FAD2-4 | cctag | 3 | 0.12 | 0.00029 | 0.60 | 0.22 | over | ABREA2HVA1 | abscisic | |
| M3 | g | | | | | | | | acid | |
| FAD2-4 | gatgg | 3 | 0.05 | 1.90E-05 | 0.15 | 0.82 | over | CAATBOX2 | | |
| M4 | cc | | | | | | | | | |
| FAD2-5 | gccac | 4 | 0.23 | 9.50E-05 | 0.20 | 0.7 | over | ABRECE1HVA22 | abscisic | |
| M1 | c | | | | | | | | acid | |
| FAD2-5 | atgtg | 6 | 0.83 | 0.00022 | 0.46 | 0.33 | over | MYCATERD1 | | drought |
| M2 | g | | | | | | | | | |
| FAD2-5 | cgagg | 3 | 0.12 | 0.00029 | 0.60 | 0.22 | over | REGION1OSOSEM | abscisic | |
| M3 | c | | | | | | | | acid | |
| FAD2-5 | caaca | 3 | 0.08 | 8.70E-05 | 0.71 | 0.15 | over | RAV1AAT | | |
| M4 | cg | | | | | | | | | |
| FAD2-5 | gccac | 3 | 0.08 | 9.50E-05 | 0.78 | 0.11 | over | ABRECE1HVA22 | abscisic | |
| M5 | ca | | | | | | | | acid | |
| FAD2-6 | ccatg | 5 | 0.28 | 1.20E-05 | 0.03 | 1.61 | over | SPHCOREZMC1 | seed | |
| M1 | c | | | | | | | | specific | |
| FAD2-6 | catgc | 4 | 0.12 | 7.70E-06 | 0.25 | 0.59 | over | RYREPEATBNNAPA | | |
| M10 | aaa | | | | | | | | | |
| FAD2-6 | catttic | 4 | 0.16 | 2.30E-05 | 0.74 | 0.13 | over | RBCSBOX3PS | | |
| M11 | c | | | | | | | | | |
| FAD2-6 | ccaac | 5 | 0.48 | 0.00014 | 0.30 | 0.52 | over | MYBPZM | | |
| M2 | c | | | | | | | | | |
| FAD2-6 | attic | 14 | 4.38 | 0.00019 | 0.39 | 0.41 | over | RBCSBOX3PS | | |
| M3 | | | | | | | | | | |
| FAD2-6 | cccaa | 5 | 0.51 | 0.00019 | 0.41 | 0.39 | over | MYBPZM | | |
| M4 | c | | | | | | | | | |
| FAD2-6 | ccatg | 4 | 0.11 | 5.60E-06 | 0.05 | 1.34 | over | SPHCOREZMC1 | | |
| M5 | ca | | | | | | | | | |
| FAD2-6 | catgc | 5 | 0.29 | 1.40E-05 | 0.12 | 0.94 | over | SORLREP5AT | | |
| M6 | aa | | | | | | | | | |
| FAD2-6 | ccatg | 4 | 0.04 | 9.20E-08 | 0.00 | 2.52 | over | SPHCOREZMC1 | | |
| M7 | caa | | | | | | | | | |
| FAD2-6 | cccca | 3 | 0.04 | 7.10E-06 | 0.23 | 0.63 | over | MYBPZM | | |
| M8 | ace | | | | | | | | | |
| FAD2-6 | ccaac | 3 | 0.04 | 7.40E-06 | 0.24 | 0.61 | over | ACIIPVPAL2 | | |
| M9 | ccc | | | | | | | | | |
| FAD2-7 | acgcg | 4 | 0.12 | 8.20E-06 | 0.02 | 1.77 | over | PE2FNTRNR1A | | UV |
| M1 | a | | | | | | | | | |
| FAD2-7 | atcgc | 4 | 0.12 | 8.20E-06 | 0.02 | 1.77 | over | PE2FNTRNR1A | | UV |
| M2 | g | | | | | | | | | |
| FAD2-7 | atgga | 9 | 1.49 | 2.50E-05 | 0.05 | 1.28 | over | MRNA3ENDTAH3 | | |
| M3 | a | | | | | | | | | |
| FAD2-7 | cttcca | 6 | 0.88 | 0.0003 | 0.62 | 0.21 | over | MRNA3ENDTAH3 | | |
| M4 | | | | | | | | | | |
| FAD2-7 | aatgtg | 5 | 0.25 | 7.00E-06 | 0.06 | 1.24 | over | MYCATERD1 | | drought |
| M5 | g | | | | | | | | | |
| FAD2-7 | acgcg | 3 | 0.04 | 7.90E-06 | 0.07 | 1.19 | over | CACGCAATGMGH3 | auxin | |
| M6 | at | | | | | | | | | |
| FAD2-7 | cacca | 4 | 0.23 | 9.00E-05 | 0.74 | 0.13 | over | POLLEN2LELAT52 | | |
| M7 | ta | | | | | | | | | |
| FAD2-8 | aggcc | 5 | 0.35 | 0.000033 | 0.07 | 1.17 | over | minus284MOTIFZMSBE1 | | |
| M1 | a | | | | | | | | | |
| FAD2-8 | gccac | 4 | 0.23 | 0.000095 | 0.20 | 0.7 | over | ABRECE1HVA22 | abscisic | |
| M2 | c | | | | | | | | acid | |
| FAD2-8 | gag-tg | 4 | 0.25 | 0.00013 | 0.26 | 0.58 | over | SORLIP5AT | | |
| M3 | c | | | | | | | | | |
| FAD2-8 | attggc | 5 | 0.5 | 0.00018 | 0.37 | 0.43 | over | CAATBOX2 | | |
| M4 | | | | | | | | | | |
| FAD2-8 | atgtg | 6 | 0.83 | 0.00022 | 0.46 | 0.33 | over | MYCATERD1 | | drought |
| M5 | g | | | | | | | | | |
| FAD2-8 | aaata | 1 | 10.61 | 0.00028 | 0.58 | 0.24 | under | TATABOX5 | | |
| M6 | a | | | | | | | | | |
| FAD2-8 | aaaaa | 1 | 10.29 | 0.00037 | 0.78 | 0.11 | under | PYRIMIDINEBOXHVEPB1 | gibberellins | |
| M7 | a | | | | | | | | | |
| FAD2-8 | gccac | 3 | 0.08 | 9.50E-05 | 0.78 | 0.11 | over | ABRECE1HVA22 | abscisic | |
| M8 | ca | | | | | | | | acid | |
| FAD2-9 | aaggg | 5 | 0.6 | 0.00038 | 0.79 | 0.1 | over | ELRE1PCPAL1 | | |
| M1 | g | | | | | | | | | |
| FAD2-9 | gcaat | 4 | 0.19 | 4.30E-05 | 0.35 | 0.46 | over | ASF1NTPARA | auxin | |
| M2 | ga | | | | | | | | | |

Multiple stresses strongly upregulate expression of SlFAD2-4 and SlFAD7—Because PPI network and promoter analysis suggested that certain members of the SlFAD2 family may be stress-responsive, we evaluated their expression patterns in tomato foliage (cv. Castlemart) in response to three different stresses: a virulent bacterial pathogen, Pseudomonas syringae pv. tomato DC3000 (Pst DC3000), an insect pest (the potato aphid, Macrosiphum euphorbiae), and mechanical wounding (Table 5). SlFAD6, which encodes a putative chloroplast-localized 412-desaturase, was also included in the expression analysis for comparison to the SlFAD2 family. SlFAD6 and the two SlFAD2 family members with Δ12-desaturases activity, SlFAD2-1 and SlFAD2-2, were all significantly down-regulated 5 days after pathogen infection, whereas SlFAD2-4, SlFAD2-5, SlFAD2-6, SlFAD2-7, and SlFAD2-9 were significantly upregulated. SlFAD2-4 and SlFAD2-7 were also upregulated by mechanical wounding and by the potato aphid, Macrosiphum euphorbiae. Foliar expression values for SlFAD2-3 and SlFAD2-8 were low, and these genes were not consistently detected in all samples. Our results indicate that the majority of SlFAD2 family members that lack in vitro Δ12-desaturase activity are upregulated by one or more stresses, whereas family members with confirmed Δ12-desaturase activity are downregulated or unresponsive to these stresses. Furthermore, of the stress-responsive members of the SlFAD2 family, SlFAD2-4 and SlFAD2-7 appear to be responsive to the broadest range of stresses, and were the only family members upregulated by aphid infestation. For these reasons, our subsequent experiments focused on SlFAD2-4 and SlFAD2-7.

TABLE 5
Gene Expression in Tomato Foliage. Expression of SlFAD2 family
members as well as another Δ-12 desaturase gene, SlFAD6,
were measured by RT-qPCR in a wild-type (WT) tomato cultivar
(cv. Castlemart) in response to three stresses: infestation by the
potato aphid (Macrosiphum euphorbiae, measured at 2 dpi),
infection by a virulent bacterial pathogen (Pseudomonas syringae
pv. tomato DC3000, abbreviated Pst, measured at 5 dpi), and
mechanical wounding (measured at 24 h after wounding). Gene
expression was also measured in a mutant with enhanced linoleic
acid content (spr2) compared to WT plants. Relative expression
values for each treatment were calculated relative to the
respective controls, normalized using the RPL2 housekeeping
gene, and analyzed by One-Way ANOVA. Single and double asterisks
represent fold changes that are statistically significant
at α = 0.1 and α = 0.05, respectively.
N ≥ 3.

| Genes | Aphid | Pst | Wounding | spr2/WT |
|---|---|---|---|---|
| SlFAD2-1 | −1.6 | −3.3* | −0.87 | −0.5 |
| SlFAD2-2 | −0.88 | −3.8* | −0.9 | 2.1 |
| SlFAD2-3 | 1.26 | —¹ | — | — |
| SlFAD2-4 | 6** | 12** | 80** | 8.78** |
| SlFAD2-5 | 1.02 | 9.5** | 1.28 | 2.1 |
| SlFAD2-6 | 1.71 | 2.1* | −0.69 | 1.3 |
| SlFAD2-7 | 8** | 37** | 3.4** | 7.5** |
| SlFAD2-8 | −0.95 | — | −0.83 | −0.97 |
| SlFAD2-9 | −1.04 | 12.7** | 9.39** | 47** |
| SlFAD6 | 1.74 | −9** | −3* | 1.68 |

¹No transcripts observed.

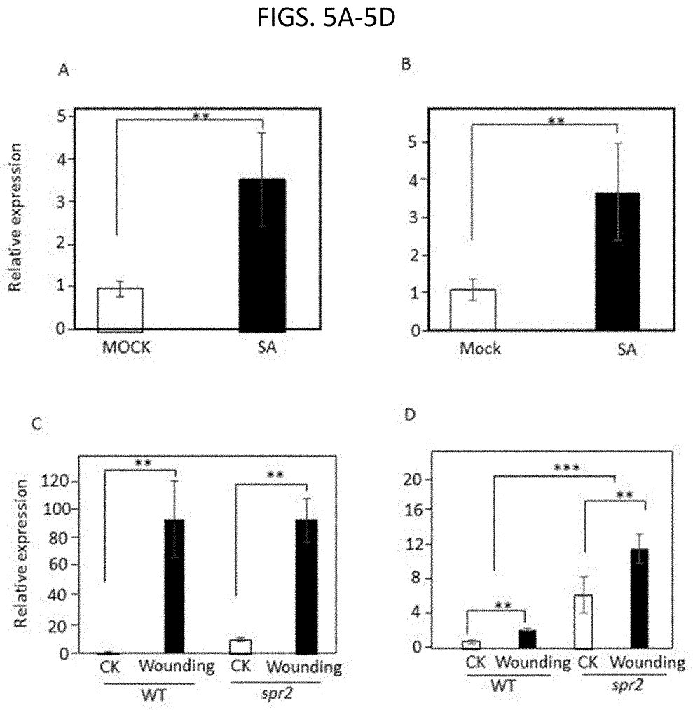

SlFAD2-4 and SlFAD2-7 are upregulated by salicylic acid, and their expression is independent of jasmonic acid synthesis—Because SlFAD2-4 and SlFAD2-7 were strongly upregulated by mechanical wounding, aphid infestation, and P. syringae infection, we also investigated how expression of these genes is influenced by salicylate- and jasmonate-signaling. These pathways are both activated by P. syringae infection and aphid infestation (Moran and Thompson, 2001; Betsuyaku et al. 2018), and jasmonates also regulate wound responses (Wasternack et al. 2006). Exogenous salicylic acid (SA) application significantly upregulated both SlFAD2-4 and SlFAD2-7 expression (FIGS. 5A-5B), indicating that both genes are SA-responsive. In contrast, constitutive SlFAD2-4 and SlFAD2-7 expression was enhanced, not inhibited, by suppressor of prosystemin-mediated responses2 (spr2) (Table 5), a mutation that inhibits jasmonic acid (JA) synthesis (Li et al. 2003). Moreover, mechanical wounding significantly upregulated SlFAD2-4 and SlFAD2-7 in spr2 as well as in wild-type plants (FIGS. 5C-5D), indicating that wound-responsive induction of these genes is independent of jasmonate signaling. Prior studies have shown that foliar expression of Fatty Acid Desaturase 7 (AtFAD7) and certain lipases in Arabidopsis can also be upregulated by wounding in mutants with impaired jasmonic acid (JA) synthesis or perception (Nishiuchi et al. 1997; Rudus et al. 2014). Thus, wound-responsive changes in lipid metabolism, including induction of divergent FAD2 family members, appears to involve JA-independent wound signaling.

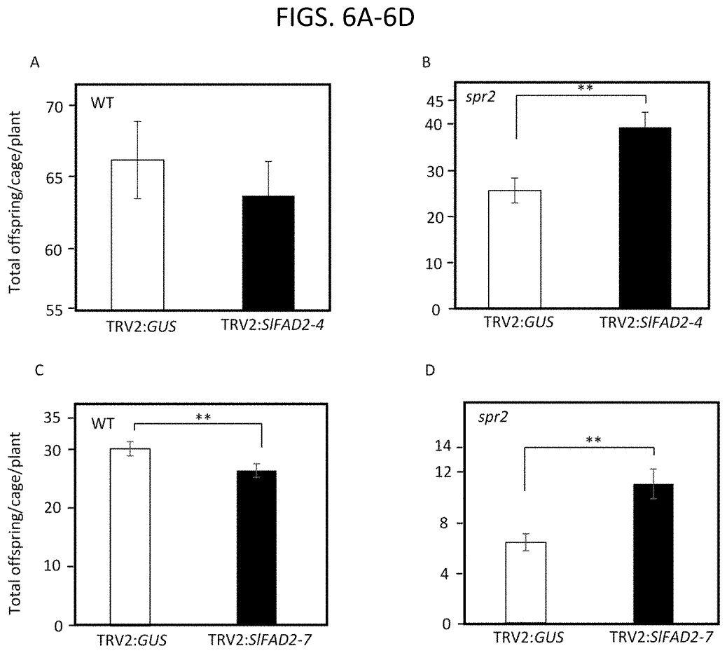

Virus Induced Gene Silencing of SlFAD2-4 or SlFAD2-7 compromises aphid resistance in the spr2 mutant, and has neutral or suppressive effects on aphids on susceptible wild-type tomato plants—The spr2 mutation, which enhances constitutive expression of the aphid-inducible and SA-responsive SlFAD2-4 and SlFAD2-7 genes (FIGS. 5A-5D), is also known to enhance aphid resistance in an SA-dependent manner (Avila et al. 2012). Therefore, we utilized virus-induced gene silencing (VIGS) to assess whether SlFAD2-4 or SlFAD2-7 contribute to aphid resistance in the spr2 mutant. Tobacco Rattle Virus (TRV) vectors were used to silence SlFAD2-4 or SlFAD2-7 in spr2, as well as in a near-isogenic, aphid-susceptible wild-type cultivar (cv. Castlemart), and population of growth of the potato aphid, Macrosiphum euphorbiae, was compared on silenced plants and plants infiltrated with vector controls (TRV:GUS). On the spr2 mutant, aphid reproduction was significantly higher on plants in which SlFAD2-4 (FIG. 6B) or SlFAD2-7 (FIG. 6D) was silenced compared to plants that received a vector control, with ≥35% (SlFAD2-4) or 45% (SlFAD2-7) increases in offspring numbers. These data indicate that SlFAD2-4 and SlFAD2-7 contribute to plant defenses against aphids in spr2. In contrast, silencing SlFAD2-4 in the near-isogenic aphid-susceptible cultivar has no significant effect on aphid numbers (FIG. 6C; p=0.53), and silencing SlFAD2-7 on this wild-type variety caused a modest but statistically significant reduction in aphid population growth (FIG. 6D). For plants infiltrated with the vector control, the wild-type genotype Castlemart supported more than twice as many aphids as spr2 (FIG. 6B compared to 6A, and FIG. 6D compared to 6C). In summary, SlFAD2-4 and SlFAD2-7 both contributed to aphid resistance in spr2, but had neutral (SlFAD2-4) or stimulatory (SlFAD2-7) effects on aphid reproduction on a wild-type, susceptible cultivar.

### DISCUSSION

FAD2 in tomato represents a multigene family, including two family members encoding confirmed Δ12-desaturases (SlFAD2-1 and SlFAD2-2), one potential pseudogene (SlFAD2-5), and six additional divergent FAD2 genes. Additionally, the tomato genome contains a single gene, SlFAD6, with homology to the AtFAD6 chloroplastic Δ12-desaturase gene.

Identifying which genes in tomato encode Δ12-desaturases is an important advance because it provides a necessary foundation for improving the fatty acid content of tomato, the world's most widely grown horticultural crop. Nearly 38 million tons of processing tomatoes were produced worldwide in 2017 (WPTC, 2019), and the US crop that year was valued at over 900 million dollars (USDA NASS, 2018). Tomato seeds represent a costly waste product of processing tomatoes, representing about 10% of fruit weight and approximately 60% of processing waste, and the use of tomato seeds for oil production has been proposed as a means of increasing the sustainability and profitability of tomato production (Schieber et al. 2001; Eller et al. 2010). Tomato seed oil is reported to be similar in taste to olive oil (Yilmaz et al. 2015), and it is also marketed as a beauty product because it contains lycopene, ß-carotene, α-tocopherol, and other antioxidants (Eller et al. 2010; Zuorro et al. 2012). However, the composition of tomato seed oil is problematic from the perspective of oxidative stability, which is important to shelf life, as well as from the perspective of stability at high temperatures, which is important for cooking oils or industrial applications. The most abundant fatty acid in tomato seed oil is linoleic acid (C18:2), which represents ˜37-57% of its total fatty content (Botinstean et al. 2015). Linoleic acid has relatively low heat stability, is prone to oxidation, and is a major contributor to rancidity in foods (Haman et al. 2017). In nearly all oil seed crops, cultivars that are low in linoleic acid and high its monunsaturated precursor oleic acid are popular, and have been developed by selecting for or engineering reduced FAD2 activity. As demonstrated in this embodiment, SlFAD2-1 and SlFAD2-2 in tomato can be targeted to enhance oil seed quality and facilitate the use of the nearly four million tons of tomato seeds that are generated annually as a waste product of processing tomato production.

Characterization of the FAD2 family in tomato is also important because of the relevance of these genes to plant stress responses. Most SlFAD2 family members were differentially regulated in response to at least one of the stresses tested here (Table 5), and SlFAD2-6 (formerly called CE1⁷19), was previously also reported to be induced by viroid infection (Gadea et al., 1996). Whereas family members with in vitro Δ-12 desaturase activity (SlFAD2-1 and SlFAD2-2) were downregulated in response to bacterial infection, five of the seven divergent members were upregulated in response to one or more biotic stresses (Table 5), suggesting that canonical and divergent FAD2 genes play distinctly different roles in plant stress responses. In Arabidopsis, the FAD2 Δ-12 desaturase contributes to adaptation to cold stress and salinity, probably by modulating membrane fluidity and ion transport across membranes (Miguel and Browse, 1994; Zhang et al. 2012). While Δ-12 desaturases influence levels of abiotic stress tolerance by modifying the physical properties of the membranes, certain divergent FAD2 enzymes contribute to biotic stress responses by participating in the synthesis of defensive secondary metabolites. The antimicrobial polyacetylenes falcarindiol and falcarinol accumulate in the vasculature of tomato in response to fungal infection (De Wit and Kodde, 1981; Elgersma et al., 1984), and data from three other plant families (Apiaceae, Asteraceae, and Araliaceae) indicate that pathogen-inducible divergent FAD2-like acetylenases synthesize necessary precursors for these phytoalexins (Kirsch et al., 1997; Cahoon et al., 2003; Busta et al., 2018). In avocado, pathogen-inducible divergent FAD2 expression is also correlated with accumulation of an antimicrobial diene, (Z,Z)-1-acetoxy-2-hydroxy-4-oxo-heneicosa-12,15-diene (Wang et al. 2004). Thus, FAD2 acetylenases and possibly other divergent FAD2 enzymes appear to contribute to the synthesis of defensive secondary metabolites in response to pathogen infection. FAD2 genes (SlFAD2-4 and SlFAD2-7) are also responsive to challenge by a piercing-sucking insect (the potato aphid), and can influence host plant susceptibility to this pest (Table 5 and FIG. 6).

The influence of SlFAD2-4 and SlFAD2-7 on plant-aphid interactions varied depending upon the tomato genotype. When either of these genes were silenced in the aphid-resistant mutant spr2, aphid infestations increased significantly, indicating that SlFAD2-4 and SlFAD2-7 contributed to aphid resistance in this genotype. In contrast, when these genes were silenced in the susceptible isogenic wild-type cultivar (cv. Castlemart), silencing had neutral or even deleterious effects on aphid population growth. The effects of individual genes on biotic interactions can often vary in different plant genotypes; for example, the efficacy of many plant genes for virus resistance are strongly influenced by their genetic background (Gallois et al. 2018). In the case of SlFAD2-4 and SlFAD2-7, the difference in their impact on aphid infestations in spr2 compared to wild type plants could be due in part to differences in their base-line expression levels in these genotypes. The constitutive transcript abundance of SlFAD2-4 and SlFAD2-7 was significantly higher in spr2 than in wild-type (Table 5), and this could potentially enhance their impact on insects in this background, particularly if these genes contribute to the production of preformed defenses. Given that SlFAD2-4 and SlFAD2-7 are both responsive to SA, their elevated basal expression levels in spr2 could perhaps be due to the enhanced salicylate signaling previously observed in this genotype (Avila et al. 2012). Another potential explanation for differences in how SlFAD2-4 and SlFAD2-7 impact aphids on spr2 versus the wild type genetic background is that these two genotypes differ in the availability of substrates for divergent FAD2 enzymes. Compared to wild-type foliage, the spr2 mutant accumulates more than three-fold higher levels of linoleic acid, one of the primary substrates for acetylenases and other divergent FAD2 enzymes (Li et al. 2003). The identity or abundance of metabolites generated by divergent FAD2 enzymes could vary in different plant genotypes depending upon substrate availability, and this could in turn influence the impact of these enzymes on biotic interactions. Similarly, we previously observed that silencing α-Dioxygenase 1 (α-DOX1), a gene involved in oxylipin synthesis, caused greater increases in aphid infestation on spr2 than on wild-type plants (Avila et al. 2013). Thus, metabolites generated from linoleic acid by divergent members of the FAD2 family as well as other enzymes such as α-DOX1 may potentially contribute to aphid resistance in spr2.

In addition to demonstrating a role for divergent FAD2 genes in insect resistance, our data also add to a growing body of evidence indicating that plant fatty acid desaturation plays important roles in plant interactions with aphids, a large and economically important group of sap-feeding insects. Infestations by the soybean aphid, Aphis glycines, reduce the abundance of polyunsaturated fatty acids in the foliage and seeds of a susceptible soybean cultivar, indicating that desaturation is responsive to aphid infestation (Kanobe et al. 2015). Desaturase activity in host plants also influences aphid population growth; loss of function of AtFAD7 or another desaturase gene, SUPPRESSOR OF SA INSENSITIVITY2 (SSI2), decreases aphid infestation on Arabidopsis, apparently by modifying defensive signaling against this pest (Pegadaraju et al. 2005; Louis et al. 2010; Avila et al. 2012). Other desaturases have a positive impact on plant defense; for example, synthesis of anacardic acid by a Δ-9 14:0-acyl carrier protein fatty acid desaturase contributes to aphid- and mite resistance in geranium (Schultz 1996). Thus, fatty acid desaturases in plants can affect aphid infestation levels by influencing defense signaling and by producing defensive secondary metabolites.

In summary, the FAD2 family in tomato includes two Δ12-desaturase genes (SlFAD2-1 and SlFAD2-2) capable of contributing to synthesis of linoleic acid in the ER, as well as five stress-responsive divergent FAD2 genes, two of which contribute to aphid resistance in a mutant (spr2) with modified fatty acid content. This data provides a foundation for manipulating oil seed content in tomato and for enhancing aphid resistance.

### Materials and Methods

Biological Materials—This study utilized the tomato cultivar Solanum lycopersicum cv. Castlemart and a mutagenized tomato line, Suppressor of prosystemin-mediated responses2 (spr2), which was developed in the Castlemart genetic background) (Li et al. 2003). Seeds for these genotypes were originally obtained from Dr. Gregg Howe (Michigan State University). All plants were germinated under stable greenhouse conditions (˜21-27° C.; 16:8 L:D photoperiod) in LC1 Sunshine potting mix (Sungro Horticulture, Belevue, W A) supplemented with 15-9-12 Osmocote Plus slow-release fertilizer (Scotts-MiracleGro Company, Marysville, Ohio), and were maintained and watered with a dilute nutrient solution containing 1000 ppm CaNO3 (Hydro Agri North America, Tampa, Fla.), 500 ppm MgSO4 (Giles Chemical Corp, Waynesville, N.C.), and 500 ppm 4-18-38 Gromore fertilizer (Gromore, Gardena, Calif.) (Avila et al., 2012). The potato aphid (Macrosiphum euphorbiae) was maintained in growth chambers (20° C.; 16 h light: 8 h dark photoperiod) on tomato seedlings (cv. Castlemart). Pseudomonas syringae pv. tomato DC3000 (Pst DC300) was provided by Dr. Yinong Yang (Pennsylvania State University), and cultured on King's B broth with rifampicin 50 μg/ml.

Identification of tomato FAD2 homologs—BLASTp (Altschul et al. 1990) was used to search for homologs of the Arabidopsis AtFAD2 predicted amino acid sequence (Genbank accession number At3G12120) in the tomato genome (ITAG release 2.40) using proteome and genome files downloaded from the SOL genomics network (SGN, solgenomics.net). For comparison, we also searched for homologs ofAtFAD6 (Genbank accession number At4G30950), which encodes a Δ-12 FAD localized to the chloroplast rather than the ER. All of the Δ-12 FAD protein sequences identified in tomato were aligned using the ClustalW algorithm with default parameters (npsa-pbil.ibcp.fr), and a phylogenetic tree was generated using default parameters in ClustalW2. Transmembrane domains were identified within these sequences using the TmPred tool from the ExPASy Bioinformatics Resource portal (Hofmann and Stoffel, 1993).

Analysis of FAD2 gene subnetwork in the STRING database—The Solanum lycopersicum protein-protein interaction (PPI) network was downloaded from the STRING database (protein.links.v11.0.txt.gz file available at string-db.org/cgi/download.pl) (Szklarczyk et al., 2017). Genes linked with each SlFAD2 gene were extracted from the network, and the overlaps among the interaction profiles of each SlFAD2 gene were quantified as Jaccard Coefficients (JC) (Brohée et al., 2008). The resulting SlFAD2 interconnected network was plotted in Cytoscape (Shannon et al. 2003). The tomato Gene Ontology (GO) was downloaded from the plant GSEA server, available at structuralbiology.cau.edu.cn/PlantGSEA/download.php (Yi et al. 2013). Given the nature and structure of the GO graph, annotations from child terms were propagated upwards to all its parent terms satisfying the ‘is a’ and ‘part of’ relationships (Ambavaram et al. 2014). Then, redundant terms (terms with high overlaps in the genes they annotate) were identified by estimating JC between every pair of GO BP terms. Within each GO term pair with JC>0.8, the GO term with the lesser number of annotations was removed. Finally, GO terms with more than 1,000 genes and less than 3 genes were removed, and hypergeometric tests were performed on the remaining terms for enrichment analysis of each set of SlFAD2-linked genes. The resulting p-values were corrected for false discoveries using Benjamini-Hochberg method and expressed as negative logarithms (Benjamini and Hochberg, 1995). These −log₁₀ values (q values) were used to create a heatmap in R using the ggplots package (rdocumentation.org/packages/ggplot2).

Analysis of Promoter motifs—The 2000 bp upstream regions of all 9 SlFAD2 genes were extracted from the Solanum lycopersicum. SL2.50.40 reference genome using the Regulatory Sequence Analysis Tools (RSAT) webserver (Nguyen et al. 2018). In each promoter, oligomers of 6, 7, and 8 bp in length were identified and the number of occurrences counted using the oligo-analysis tool in RSAT (rsat.eead.csic.es/plants/oligo-analysis_form.cgi). The expected number of occurrences was calculated from the predefined background frequencies model consisting of all upstream regions, clipping overlaps with upstream ORFs. The probability of having at least the number of observed occurrences of each motif (over-representation) or less than the observed number of occurrences (under) was calculated using the binomial formula. The resulting p-values were converted to E values, and the −log₁₀ (E value) was expressed as the significance index of identified DNA motifs. Motifs reported with significance index >0 were then compared to known plant CREs listed in the PLACE database using the STAMP server (Higo et al. 1999); Mahoney et al. 2007).

Heterologous Expression of SlFAD2 genes from tomato in yeast—For each of the nine SlFAD2 homologs identified in tomato, primers were designed to target the entire open reading frame (Table 2), which was amplified from cDNA synthesized from total RNA extracted from foliage of the tomato cultivar Castlemart. A clone of the AtFAD2 gene from Arabidopisis (clone U12792) was obtained from the Arabidopsis information resource (TAIR). The blunt-end PCR products containing the tomato cDNAs were cloned into the pENTR TOPO vector (Invitrogen, Carlsbad, Calif., USA), and then cloned into the destination vector pYES-DEST52 (Thermo Fisher Scientific, Waltham Mass.) using Gateway cloning recombination technology (Invitrogen, LA Jolla, Calif., USA). The pYES-DEST52 expression vector includes the GALACTOKINASEI (GALL) promoter for inducible gene expression in yeast. The resulting plasmids and a vector control consisting of the pYES-DEST52 vector containing a β-glucuronidase (GUS) cDNA insert were introduced into Saccharomyces cerevisiae INVSc1 cells (Invitrogen, Carlsbad, Calif., USA). Transformants were first grown in minimal medium lacking uracil and containing glucose at 28° C. After overnight culture, the cells were collected, washed one time with water, and diluted to an OD of 0.4 in minimal media containing galactose to induce expression of the transgenes.

Fatty acid analysis—The fatty acid profiles of yeast lines expressing SlFAD2 homologs were analyzed by gas chromatography-mass spectrometry (GC-MS) of fatty acid methyl esters (FAMEs) using protocols adapted from Cao and coworkers (2013). In brief, 10 ml saturated cultures were grown for 3 days at 15° C., and cells were collected, washed three times with water, pelleted, and dried under vacuum. Lipids were extracted and transmethylated as follows. 2 mL of 1 N methanolic HCl was added to the materials, sealed in 8 ml glass tube, and heated to 80° C. for lhr. After cooling on ice, 2 mL of 0.9% NaCl was added, and the mixture was extracted three times with 2 mL of hexane. For fatty acid extraction and FAMEs preparation from plant tissues, fatty acids were extracted using a method described by Miguel and Browse with some modifications (J. Biol. Chem. 267 (1992):1502-1509). Approximately 50 mg of leaf tissue was used for the extractions. The fatty acids were simultaneously extracted and esterified in 2.5% (v/v) H₂SO₄ in methanol at 80° C. for 90 minutes in a screw cap kimbal vial. The organic phase containing fatty acids was separated from the rest by adding 0.9 percent NaCl and 1 ml hexane and centrifuging. The organic phase containing fatty acids was purified on a Silicic acid column and finally dissolved in 150 ul hexane containing 0.5% butylated hydroxytoulene. All FAMEs samples were analyzed using GC-Agilent 6890N/MS-Agilent 5937 with FAMEWAX columns (30 m×0.25 μm film thickness; Restek, Bellefonte, Pa.). 1 ul of sample was injected and 14:1 of split ratio was applied. The oven temperature was raised from 130 to 225° C. at a rate of 7° C./min and then maintained at 225° C. for 12 min. Peaks were identified by comparing the retention times with those of the corresponding standards (Nu-Chek-Prep Inc., Elysian, Minn.).

Stress Challenge and Salicylic Acid Treatment—In order to test the influence of stresses on SlFAD2 gene expression, 4-week-old tomato plants (cv. Castlemart) were challenged with Pst DC3000, potato aphid infestation, or mechanical wounding. Pst DC3000 inoculation was performed according to previously described protocols (Uppalapati et al., 2011). In brief, plants were spray-inoculated with a Pst DC3000 bacterial suspension (OD600=0.005) in 10 mM Mgcl₂ containing 0.025% Silwet L-77 (OSI specialties Inc., Danbury, Conn., USA). Plants were then incubated in growth chambers at 100% RH for the first 24 hr and at 70% RH for the remainder of the experiment. Control plants were mock-inoculated using the same protocols with corresponding solutions that lacked bacteria (6 replicate plants/treatment). Leaf samples for gene expression analysis were flash-frozen in liquid nitrogen five days after inoculation, the earliest time point to observe consistent symptoms of infection. To measure gene expression in response to infestation by the potato aphid, aphids were confined to cloth sleeve cages (each enclosing 2 leaflets) placed on the second and third leaf below the meristem of 4 week old plants (2 cages/plant; 60 aphids/cage), while mock-challenged plants received empty cages (5 replicate plants/treatment). Cages and aphids were removed 48 h after infestation, and leaves were then flash-frozen for RNA extraction. To measure gene expression in response to mechanical wounding, a leaf of each plant was crushed with a hemostat to produce a row of punctures that crossed the midvein. Leaves were harvested and flash-frozen 24 h after wounding. In order to assess gene expression in response to salicylic acid (SA), the foliage of 4-week old tomato plants was sprayed with 100 μM SA in 0.1% ethanol. Equal volumes of 0.1% ethanol solution were applied to mock controls (8 replicate plants/treatment). Leaves were harvested and flash-frozen 24 h after hormone application.

Gene expression analysis—Tissue for gene expression experiments was collected from the youngest leaf that was fully expanded, which was typically the third or fourth leaf below the meristem. After flash freezing, total RNA was purified from tomato foliage using TRIzol according to the manufacturer's instructions (Invitrogen, Carlsbad, Calif., U.S.A.). First-strand cDNA was synthesized from total RNA using the Superscript III First-strand synthesis system and oligo dT primers (Invitrogen, Carlsbad, Calif., U.S.A.). Gene expression analysis was performed by RT-qPCR using a StepOnePlus real time PCR system (Applied Biosystems, Foster City, Calif., U.S.A.) and the QuantiTect SYBR Green PCR kit (Qiagen, Valencia, Calif., U.S.A.). Each experiment included at least three biological replicates per treatment, and two technical replicates per biological sample. Gene-specific primer sets were designed to measure expression of each of the 9 SlFAD2 genes, as well as SlFAD6 for comparison (see Table 2 for primer sequences). RIBOSOMAL PROTEIN L2 (RPL2, Genbank accession number GBX64562) was included as endogenous housekeeping control gene, and quantified using the following primers: forward (5′-GAGGGCGTACTGAGAAACCA-3′; SEQ ID NO:41) and reverse (5′-CTTTTGTCCAGGAGGTGCAT-3′; SEQ ID NO:42). The PCR conditions used for all primer sets were 95° C. for 3 min, followed by 40 cycles of 94° C. for 15 s, 55° C. for 30 s, 72° C. for 30 s, with data acquisition at the end of each cycle, and a final data acquisition step to generate melting curves from 65° C. to 95° C. every 0.3° C. The efficiency of amplification of each primer set was calculated by applying the formula “E=10[−1/Ct slope]” to data generated using serial dilutions of a sample pooled from aliquots of all cDNA samples (Rasmussen 2000). Data for each sample was normalized to the expression levels of the endogenous control RPL2, and gene expression levels in treated plants were calculated relative to the untreated wild-type control group in each experiment.

Virus Induced Gene Silencing (VIGS)—Expression of SlFAD2-7 (locus name Solyc12g049030) was transiently suppressed in tomato foliage using a Tobacco Rattle Virus (TRV) vector for virus induced gene silencing, according to previously described protocols (Liu et al., 2002). The TRV1 and TRV2 VIGS vectors were kindly provided by S. P. Dinesh-Kumar (University of California, Davis). A 440 bp fragment of SlFAD2-7 (bases 442-881 in the SOL Genomics Network sequence Solyc12g049030.1.1) was amplified by using a forward primer containing an EcoRI restriction site (5′-CGGGAATTCACTGCTTCCCTTGAGAACGA-3′; SEQ ID NO:43) and a reverse primer containing an Xhol restriction site (5′-CGGCTCGAG GCTCCTCGTAGCCAATTCCA-3′; SEQ ID NO:44). The PCR-amplified fragment was cloned into the TRV2 vector pYL156 and electroporated into Agrobacterium tumefaciens GV3101. Cultures of A. tumefaciens harboring TRV1 or the TRV2:SlFAD2-7 construct were grown overnight at 28° C. in lysogeny broth (LB) with 50 μg kanamycin and 50 μg rifampicin. One ml overnight cultures were transferred into 40 ml of fresh LB and shaken overnight at 28° C. Bacterial cells were harvested and resuspended in 40 ml induction medium (10 mM MgCl₂, 10 mM 2-(N-morpholino)ethanesulfonic acid, pH 5.6) supplemented with 150 uM acetosyringone and shaken at room temperature for 5 hrs. Bacterial cultures containing TRV1 and TRV2:SlFAD2-7 were mixed in equal ratios (OD 600=1.0), and infiltrated into tomato plants at the two-leaf stage (˜10 days after germination) using a needleless syringe (Velasquez et al. 2009). Additional sets of plants were also infiltrated with a mix of TRV1 and TRV:GUS, or TRV1 and TRV:LePDS. The TRV:GUS construct targets a β-Glucuronidase gene that is absent in tomato, and it is a useful negative control because it is similar in size to our experimental construct, but does not cause any silencing (Wu et al. 2011). TRV:LePDS silences PHYTOENE DESATURASE and results in photobleaching of the leaves, providing a useful indicator of the timing of silencing (Ruiz et al., 1998). After Agroinfiltration, plants were maintained at 20° C., and 50% relative humidity with a 16 h light: 8 h dark photoperiod (220 μM/m²/sec light intensity) in a growth chamber (Conviron, Winnepeg, Canada). Plants infiltrated with TRV:LePDS were monitored visually to assess the onset of silencing, and all other plants were used for bioassays only after the TRV:LePDS plants showed extensive photobleaching (3 to 3.5 weeks after infiltration). SlFAD2-1 was silenced by the same techniques, cloning a 441 bp fragment of SlFAD2-1 (bases 1025-1552 bp in the SOL Genomics Network sequence Solyc01g006430) into the TRV vector.

Assay to Measure Aphid Infestation on VIGS-treated Plants—Adult potato aphids of uniform age (collected within 24 h after emergence to adulthood) were confined to individual leaflets using clip cages (18 to 20 replicate plants per treatment group; 3 cages/plant; 4 aphids/cage). Cages were placed on the youngest leaf that was fully expanded, which was typically the third or fourth leaf below the meristem. Four to five days after inoculation, total offspring were counted. Individual plants were treated as biological replicates, and replicate cages on the same plant were treated as subsamples, and averaged before analysis. Silencing of SlFAD2-7 was confirmed in a sub-set of plants (20 replicates/treatment group) at the end of the bioassay. After aphid removal, three leaf punches (10 mm) per infected leaf (3 leaves/plant) were collected and flash-frozen. Leaf discs were pooled into a single sample per plant, and RNA extraction and RT-qPCR analysis of SlFAD2-7 expression was performed as described above.

Statistical Analysis.

Bioassay data and gene expression data were analyzed by One-way ANOVA (for experiments with a single variable) or Two-way ANOVA (for experiments with a full-factorial combination of two variables) using JMP® Pro 13 (SAS Institute Inc.).

### REFERENCES

Aguilar P S, de Mendoza D (2006) Control of fatty acid desaturation: a mechanism conserved from bacteria to humans. Mol Microbiol 62: 1507-1514
Altschul S F, Gish W, Miller W, Myers E W, Lipman D J (1990) Basic local alignment search tool. J Mol Biol 215: 403-410
Ambavaram M M, Basu S, Krishnan A, Ramegowda V, Batlang U, Rahman L, Baisakh N, Pereira A (2014) Coordinated regulation of photosynthesis in rice increases yield and tolerance to environmental stress. Nat Commun 5: 5302
Avila C A, Arevalo-Soliz L M, Jia L, Navarre D A, Chen Z, Howe G A, Meng Q W, Smith J E, Goggin F L (2012) Loss of function of FATTY ACID DESATURASE7 in tomato enhances basal aphid resistance in a salicylate-dependent manner. Plant Physiol 158: 2028-2041
Avila C A, Arevalo-Soliz L M, Lorence A, Goggin F L (2013) Expression of alpha-DIOXYGENASE 1 in tomato and Arabidopsis contributes to plant defenses against aphids. Mol Plant Microbe Interact 26: 977-986
Benjamini Y, Hochberg Y (1995) Controlling the False Discovery Rate: A Practical and Powerful Approach to Multiple Testing. Journal of the Royal Statistical Society. Series B (Methodological) 57: 289-300
Betsuyaku S, Katou S, Takebayashi Y, Sakakibara H, Nomura N, Fukuda H (2018) Salicylic Acid and Jasmonic Acid Pathways are Activated in Spatially Different Domains Around the Infection Site During Effector-Triggered Immunity in Arabidopsis thaliana. Plant Cell Physiol 59: 8-16
Botinetean C, Gruia A T, Jianu I (2015) Utilization of seeds from tomato processing wastes as raw material for oil production. Journal of Material Cycles and Waste Management 17: 118-124
Broadwater J A, Whittle E, Shanklin J (2002) Desaturation and hydroxylation. Residues 148 and 324 of Arabidopsis FAD2, in addition to substrate chain length, exert a major influence in partitioning of catalytic specificity. J Biol Chem 277: 15613-15620
Brohee S, Faust K, Lima-Mendez G, Sand O, Janky R, Vanderstocken G, Deville Y, van Helden J (2008) NeAT: a toolbox for the analysis of biological networks, clusters, classes and pathways. Nucleic Acids Res 36: 444
Broun P, Boddupalli S, Somerville C (1998a) A bifunctional oleate 12-hydroxylase: desaturase from Lesquerella fendleri. Plant J 13: 201-210
Broun P, Shanklin J, Whittle E, Somerville C (1998b) Catalytic plasticity of fatty acid modification enzymes underlying chemical diversity of plant lipids. Science 282: 1315-1317
Browse J, Kunst L, Anderson S, Hugly S, Somerville C (1989) A mutant of Arabidopsis deficient in the chloroplast 16:1/18:1 desaturase. Plant Physiol 90: 522-529
Buhr T, Sato S, Ebrahim F, Xing A, Zhou Y, Mathiesen M, Schweiger B, Kinney A, Staswick P, Tom Clemente (2002) Ribozyme termination of RNA transcripts down-regulate seed fatty acid genes in transgenic soybean. Plant J 30: 155-163
Busta L, Yim W C, LaBrant E W, Wang P, Grimes L, Malyszka K, Cushman J C, Santos P, Kosma D K, Cahoon E B (2018) Identification of Genes Encoding Enzymes Catalyzing the Early Steps of Carrot Polyacetylene Biosynthesis. Plant Physiol 178: 1507-1521
Cahoon E B, Carlson T J, Ripp K G, Schweiger B J, Cook G A, Hall S E, Kinney A J (1999) Biosynthetic origin of conjugated double bonds: production of fatty acid components of high-value drying oils in transgenic soybean embryos. Proc Natl Acad Sci USA 96: 12935-12940
Cahoon E B, Kinney A J (2004) Dimorphecolic acid is synthesized by the coordinate activities of two divergent Delta12-oleic acid desaturases. J Biol Chem 279: 12495-12502
Cahoon E B, Ripp K G, Hall S E, Kinney A J (2001) Formation of conjugated delta8,delta10-double bonds by delta12-oleic-acid desaturase-related enzymes: biosynthetic origin of calendic acid. J Biol Chem 276: 2637-2643
Cahoon E B, Schnurr J A, Huffman E A, Minto R E (2003) Fungal responsive fatty acid acetylenases occur widely in evolutionarily distant plant families. Plant J 34: 671-683
Canonne J, Froidure-Nicolas S, Rivas S (2011) Phospholipases in action during plant defense signaling. Plant Signal Behav 6: 13-18
Cao S, Zhou X R, Wood C C, Green A G, Singh S P, Liu L, Liu Q (2013) A large and functionally diverse family of Fad2 genes in safflower (Carthamus tinctorius L.). BMC Plant Biol 13: 5
Chapman K D, Austin-Brown S, Sparace S A, Kinney A J, Ripp K G, Pirtle I L, Pirtle R M (2001) Transgenic cotton plants with increased seed oleic acid content. J Am Oil Chem Soc 78: 941-947
Chaturvedi R, Krothapalli K, Makandar R, Nandi A, Sparks A A, Roth M R, Welti R, Shah J (2008) Plastid omega3-fatty acid desaturase-dependent accumulation of a systemic acquired resistance inducing activity in petiole exudates of Arabidopsis thaliana is independent ofjasmonic acid. Plant J 54: 106-117
Chehab E W, Perea J V, Gopalan B, Theg S, Dehesh K (2007) Oxylipin Pathway in Rice and Arabidopsis. Journal of Integrative Plant Biology 49: 43-51
Chi X, Yang Q, Pan L, Chen M, He Y, Yang Z, Yu S (2011) Isolation and characterization of fatty acid desaturase genes from peanut (Arachis hypogaea L.). Plant Cell Rep 30: 1393-1404
Clemente T E, Cahoon E B (2009) Soybean oil: genetic approaches for modification of functionality and total content. Plant Physiol 151: 1030-1040
Dar A A, Choudhury A R, Kancharla P K, Arumugam N (2017) The FAD2 Gene in Plants: Occurrence, Regulation, and Role. Front Plant Sci 8: 1789
Defibaugh-Chavez, S L H 2008. Plant defenses against piercing-sucking insects and the effects of Mi-mediated resistance on Macrosiphum euphorbiae. University of Arkansas, Ph.D. Dissertation.
De Wit, P J G M., & Kodde, E (1.981) Induction of polyacetylenic phytoalexins in Lycopersicon esculentum after inoculation with Cladosporium fulvum (syn. Fulvia fulva). Physiol Plant Pathol, 18, 143-148.
Dominguez T, Hernandez M L, Pennycooke J C, Jimenez P, Martinez-Rivas J M, Sanz C, Stockinger E J, Sanchez-Serrano J J, Sanmartin M (2010) Increasing omega-3 desaturase expression in tomato results in altered aroma profile and enhanced resistance to cold stress. Plant Physiol 153: 655-665
Dyer J M, Chapital D C, Kuan J W, Mullen R T, Turner C, McKeon T A, Pepperman A B (2002) Molecular Analysis of a Bifunctional Fatty Acid Conjugase/Desaturase from Tung. Implications for the Evolution of Plant Fatty Acid Diversity. Plant Physiol 130: 2027-2038
Elgersma, D M, Weijman, A C M., Roeymans, H J, van Eijk, G W (1984) Occurrence of falcarinol and falcarindiol in tomato plants after infection with Verticillium alboatrum and charcterization of four phytoalexins by capillary gas chromatography-mass spectrometry. Phytopathol Z 0.1 Phytopathol 109: 237-240.
Ekengren S K, Liu Y, Schiff M, Dinesh-Kumar S P, Martin G B (2003) Two MAPK cascades, NPR1, and TGA transcription factors play a role in Pto-mediated disease resistance in tomato. Plant J 36: 905-917
Eller F J, Moser J K, Kenar J A, Taylor S L (2010) Extraction and Analysis of Tomato Seed Oil. J Am Oil Chem Soc 87: 755-762
Falcone D L, Gibson S, Lemieux B, Somerville C (1994) Identification of a gene that complements an Arabidopsis mutant deficient in chloroplast omega 6 desaturase activity. Plant Physiol 106: 1453-1459
Farmer E E (1994) Fatty acid signalling in plants and their associated microorganisms. Plant Mol Biol 26: 1423-1437
Feng J, Dong Y, Liu W, He Q, Daud M K, Chen J, Zhu S (2017) Genome-wide identification of membrane-bound fatty acid desaturase genes in Gossypium hirsutum and their expressions during abiotic stress. Sci Rep 7: 45711
Gadea J, Mayda M E, Conejero V, Vera P (1996) Characterization of defense-related genes ectopically expressed in viroid-infected tomato plants. Mol Plant Microbe Interact 9: 409-415
Gagne S J, Reed D W, Gray G R, Covello P S (2009) Structural control of chemoselectivity, stereoselectivity, and substrate specificity in membrane-bound fatty acid acetylenases and desaturases. Biochemistry 48: 12298-12304
Gallois J, Moury B, German-Retana S (2018) Role of the Genetic Background in Resistance to Plant Viruses. International Journal of Molecular Sciences 19:
Haman N, Romano A, Asaduzzaman M, Ferrentino G, Biasioli F, Scampicchio M (2017) A microcalorimetry study on the oxidation of linoleic acid and the control of rancidity. Talanta 164: 407-412
Hamdan Y A S, Garcia-Moreno M J, Fernandez-Martinez J M, Velasco L, Perez-Vich B (2012) Mapping of major and modifying genes for high oleic acid content in safflower. Mol Breed 30: 1279-1293
Haun W, Coffman A, Clasen B M, Demorest Z L, Lowy A, Ray E, Retterath A, Stoddard T, Juillerat A, Cedrone F, Mathis L, Voytas D F, Zhang F (2014) Improved soybean oil quality by targeted mutagenesis of the fatty acid desaturase 2 gene family. Plant Biotechnol J 12: 934-940
Heppard E P, Kinney A J, Stecca K L, Miao G H (1996) Developmental and growth temperature regulation of two different microsomal omega-6 desaturase genes in soybeans. Plant Physiol 110: 311-319
Hernandez M L, Mancha M, Martinez-Rivas J M (2005) Molecular cloning and characterization of genes encoding two microsomal oleate desaturases (FAD2) from olive. Phytochemistry 66: 1417-1426
Higo K, Ugawa Y, Iwamoto M, Korenaga T (1999) Plant cis-acting regulatory DNA elements (PLACE) database: 1999. Nucleic Acids Res 27: 297-300
Hitz W D, Carlson T J, Booth J R, Jr, Kinney A J, Stecca K L, Yadav N S (1994) Cloning of a higher-plant plastid omega-6 fatty acid desaturase cDNA and its expression in a cyanobacterium. Plant Physiol 105: 635-641
Hofmann K, Stoffel W (1993) TMBASE—A database of membrane spanning protein segments. Biol Chem Hoppe-Seyler 374: 166
Jump D B (2002) Dietary polyunsaturated fatty acids and regulation of gene transcription. Curr Opin Lipidol 13: 155-164 Jung S, Powell G, Moore K, Abbott A (2000) The high oleate trait in the cultivated peanut [Arachis hypogaea L]. II. Molecular basis and genetics of the trait. Mol Gen Genet 263: 806-811
Kajiwara S, Shirai A, Fujii T, Toguri T, Nakamura K, Ohtaguchi K (1996) Polyunsaturated fatty acid biosynthesis in Saccharomyces cerevisiae: expression of ethanol tolerance and the FAD2 gene from Arabidopsis thaliana. Appl Environ Microbiol 62: 4309-4313
Kang J, Snapp A R, Lu C (2011) Identification of three genes encoding microsomal oleate desaturases (FAD2) from the oilseed crop Camelina sativa. Plant Physiol Biochem 49: 223-229
Kanobe C, McCarville M T, O'Neal M E, Tylka G L, MacIntosh GC (2015) Soybean Aphid Infestation Induces Changes in Fatty Acid Metabolism in Soybean. PLoS One 10: e0145660
Kargiotidou A, Deli D, Galanopoulou D, Tsaftaris A, Farmaki T (2008) Low temperature and light regulate delta 12 fatty acid desaturases (FAD2) at a transcriptional level in cotton (Gossypium hirsutum). J Exp Bot 59: 2043-2056
Kirsch C, Hahlbrock K, Somssich I E (1997) Rapid and transient induction of a parsley microsomal delta 12 fatty acid desaturase mRNA by fungal elicitor. Plant Physiol 115: 283-289
Knutzon D S, Thompson G A, Radke S E, Johnson W B, Knauf V C, Kridl J C (1992) Modification of Brassica seed oil by antisense expression of a stearoyl-acyl carrier protein desaturase gene. Proc Natl Acad Sci USA 89: 2624-2628
Kunst L, Browse J, Somerville C (1989) A mutant of Arabidopsis deficient in desaturation of palmitic Acid in leaf lipids. Plant Physiol 90: 943-947
Lakhssassi N, Zhou Z, Liu S, Colantonio V, AbuGhazaleh A, Meksem K (2017) Characterization of the FAD2 Gene Family in Soybean Reveals the Limitations of Gel-Based TILLING in Genes with High Copy Number. Front Plant Sci 8: 324
Lee K R, Kim S H, Go Y S, Jung S M, Roh K H, Kim J B, Suh M C, Lee S, Kim H U (2012) Molecular cloning and functional analysis of two FAD2 genes from American grape (Vitis labrusca L.). Gene 509: 189-194
Lee M, Lenman M, Banas A, Bafor M, Singh S, Schweizer M, Nilsson R, Liljenberg C, Dahlqvist A, Gummeson P O, Sjodahl S, Green A, Stymne S (1998) Identification of non-heme diiron proteins that catalyze triple bond and epoxy group formation. Science 280: 915-918
Lee Y, Park W, Kim K, Jang Y, Lee J, Cha Y, Moon Y, Song Y, Lee K (2018) EMS-induced mutation of an endoplasmic reticulum oleate desaturase gene (FAD2-2) results in elevated oleic acid content in rapeseed (Brassica napus L.). Euphytica 214: 28
Li A, Zhang R, Pan L, Tang L, Zhao G, Zhu M, Chu J, Sun X, Wei B, Zhang X, Jia J, Mao L (2011) Transcriptome analysis of H2O2-treated wheat seedlings reveals a H2O2-responsive fatty acid desaturase gene participating in powdery mildew resistance. PLoS One 6: e28810
Li C, Liu G, Xu C, Lee G I, Bauer P, Ling H Q, Ganal M W, Howe G A (2003) The tomato suppressor of prosystemin-mediated responses2 gene encodes a fatty acid desaturase required for the biosynthesis of jasmonic acid and the production of a systemic wound signal for defense gene expression. Plant Cell 15: 1646-1661
Li L, Wang X, Gai J, Yu D (2007) Molecular cloning and characterization of a novel microsomal oleate desaturase gene from soybean. J Plant Physiol 164: 1516-1526
Lim G H, Singhal R, Kachroo A, Kachroo P (2017) Fatty Acid- and Lipid-Mediated Signaling in Plant Defense. Annu Rev Phytopathol 55: 505-536
Liu Q, Brubaker C L, Green A G, Marshall D R, Sharp P J, Singh S P (2001) Evolution of the FAD2-1 fatty acid desaturase 5′ UTR intron and the molecular systematics of Gossypium (Malvaceae). Am J Bot 88: 92-102
Liu Y, Schiff M, Dinesh-Kumar S P (2002) Virus-induced gene silencing in tomato. Plant J 31: 777-786
Liu Y, Schiff M, Marathe R, Dinesh-Kumar S P (2002) Tobacco Rarl, EDS1 and NPR1/NIM1 like genes are required for N-mediated resistance to tobacco mosaic virus. Plant J 30: 415-429
López Y, Nadaf H L, Smith O D, Connell J P, Reddy A S, Fritz A K (2000) Isolation and characterization of the Δ12-fatty acid desaturase in peanut (Arachis hypogaea L.) and search for polymorphisms for the high oleate trait in Spanish market-type lines. Theor Appl Genet 101: 1131-1138
Louis J, Leung Q, Pegadaraju V, Reese J, Shah J (2010) PAD4-dependent antibiosis contributes to the ssi2-conferred hyper-resistance to the green peach aphid. Mol Plant Microbe Interact 23: 618-627
Lozinsky S, Yang H, Forseille L, Cook G R, Ramirez-Erosa I, Smith M A (2014) Characterization of an oleate 12-desaturase from Physaria fendleri and identification of 5′UTR introns in divergent FAD2 family genes. Plant Physiol Biochem 75: 114-122
Mahony S, Benos P V (2007) STAMP: a web tool for exploring DNA-binding motif similarities. Nucleic Acids Res 35: 253
Martinez-Rivas J M, Garcia-Diaz M T, Mancha M (2000) Temperature and oxygen regulation of microsomal oleate desaturase (FAD2) from sunflower. Biochem Soc Trans 28: 890-892
McCartney A W, Dyer J M, Dhanoa P K, Kim P K, Andrews D W, McNew J A, Mullen R T (2004) Membrane-bound fatty acid desaturases are inserted co-translationally into the ER and contain different ER retrieval motifs at their carboxy termini. The Plant Journal 37: 156-173
Mei C, Michaud M, Cussac M, Albrieux C, Gros V, Marechal E, Block M A, Jouhet J, Rebeille F (2015) Levels of polyunsaturated fatty acids correlate with growth rate in plant cell cultures. Sci Rep 5: 15207
Mikkilineni V, Rocheford T R (2003) Sequence variation and genomic organization of fatty acid desaturase-2 (fad2) and fatty acid desaturase-6 (fad6) cDNAs in maize. Theor Appl Genet 106: 1326-1332
Miguel M F, Browse J A (1994) High-Oleate Oilseeds Fail to Develop at Low Temperature. Plant Physiol 106: 421-427
Moran P J, Thompson G A (2001) Molecular responses to aphid feeding in Arabidopsis in relation to plant defense pathways. Plant Physiol 125: 1074-1085
Nguyen N T T, Contreras-Moreira B, Castro-Mondragon J A, Santana-Garcia W, Ossio R, Robles-Espinoza C D, Bahin M, Collombet S, Vincens P, Thieffry D, van Helden J, Medina-Rivera A, Thomas-Chollier M (2018) RSAT 2018: regulatory sequence analysis tools 20th anniversary. Nucleic Acids Res 46: W214
Nishiuchi T, Hamada T, Kodama H, Iba K (1997) Wounding changes the spatial expression pattern of the arabidopsis plastid omega-3 fatty acid desaturase gene (FAD7) through different signal transduction pathways. Plant Cell 9: 1701-1712
Ohlrogge J, Browse J (1995) Lipid biosynthesis. Plant Cell 7: 957-970
Okada S, Zhou X R, Damcevski K, Gibb N, Wood C, Hamberg M, Haritos V S (2013) Diversity of Deltal2 fatty acid desaturases in santalaceae and their role in production of seed oil acetylenic fatty acids. J Biol Chem 288: 32405-32413
Okuley J, Lightner J, Feldmann K, Yadav N, Lark E, Browse J (1994) Arabidopsis FAD2 gene encodes the enzyme that is essential for polyunsaturated lipid synthesis. Plant Cell 6: 147-158
Pegadaraju V, Knepper C, Reese J, Shah J (2005) Premature leaf senescence modulated by the Arabidopsis PHYTOALEXIN DEFICIENT4 gene is associated with defense against the phloem-feeding green peach aphid. Plant Physiol 139: 1927-1934
Peng Q, Hu Y, Wei R, Zhang Y, Guan C, Ruan Y, Liu C (2010) Simultaneous silencing of FAD2 and FAE1 genes affects both oleic acid and erucic acid contents in Brassica napus seeds. Plant Cell Rep 29: 317-325
Pham A T, Shannon J G, Bilyeu K D (2012) Combinations of mutant FAD2 and FAD3 genes to produce high oleic acid and low linolenic acid soybean oil. Theor Appl Genet 125: 503-515
Pirtle I L, Kongcharoensuntorn W, Nampaisansuk M, Knesek J E, Chapman K D, Pirtle R M (2001) Molecular cloning and functional expression of the gene for a cotton Delta-12 fatty acid desaturase (FAD2). Biochim Biophys Acta 1522: 122-129
Rasmussen R (2000) Quantification on the lightcycler. In rapid cycle real-time PCR (S Meuer, C Wittwer, K Nakagawara, ed). Methods and Applications. Springer, Heidelberg, pp. 21-34.
Ray T K, Holly S P, Knauft D A, Abbott A G, Powell G L (1993) The primary defect in developing seed from the high oleate variety of peanut (Arachis hypogaea L.) is the absence of Δ12-desaturase activity. Plant Science 91: 15-21
Reed D W, Savile C K, Qiu X, Buist P H, Covello P S (2002) Mechanism of 1,4-dehydrogenation catalyzed by a fatty acid (1,4)-desaturase of Calendula officinalis. Eur J Biochem 269: 5024-5029
Rudus I, Terai H, Shimizu T, Kojima H, Hattori K, Nishimori Y, Tsukagoshi H, Kamiya Y, Seo M, Nakamura K, Kepczynski J, Ishiguro S (2014) Wound-induced expression of DEFECTIVE IN ANTHER DEHISCENCE1 and DAD1-like lipase genes is mediated by both CORONATINE INSENSITIVE1-dependent and independent pathways in Arabidopsis thaliana. Plant Cell Rep 33: 849-860
Ruiz M T, Voinnet O, Baulcombe D C (1998) Initiation and maintenance of virus-induced gene silencing. Plant Cell 10: 937-946
Saeki K, Matsumoto K, Kinoshita M, Suzuki I, Tasaka Y, Kano K, Taguchi Y, Mikami K, Hirabayashi M, Kashiwazaki N, Hosoi Y, Murata N, Iritani A (2004) Functional expression of a Deltal2 fatty acid desaturase gene from spinach in transgenic pigs. Proc Natl Acad Sci USA 101: 6361-6366
Schieber A, Stintzing F C, Carle R (2001) By-products of plant food processing as a source of functional compounds—recent developments. Trends in Food Science & Technology 12: 401-413
Schierholt A, Becker H C, Ecke W (2000) Mapping a high oleic acid mutation in winter oilseed rape (Brassica napus L.). Theor Appl Genet 101: 897-901
Schultz D J, Cahoon E B, Shanklin J, Craig R, Cox-Foster D L, Mumma R O, Medford J I (1996) Expression of a delta 9 14:0-acyl carrier protein fatty acid desaturase gene is necessary for the production of omega 5 anacardic acids found in pest-resistant geranium (Pelargonium xhortorum). Proc Natl Acad Sci USA 93: 8771-8775
Schuppert G F, Tang S, Slabaugh M B, Knapp S J (2006) The Sunflower High-oleic Mutant 01 Carries Variable Tandem Repeats of FAD2-1, a Seed-specific Oleoyl-phosphatidyl Choline Desaturase. Mol Breed 17: 241-256
Shahidi F, Zhong Y (2010) Lipid oxidation and improving the oxidative stability. Chem Soc Rev 39: 4067-4079
Shanklin J, Cahoon E B (1998) Desaturation and Related Modifications of Fatty Acidsl. Annu Rev Plant Physiol Plant Mol Biol 49: 611-641
Shannon P, Markiel A, Ozier O, Baliga N S, Wang J T, Ramage D, Amin N, Schwikowski B, Ideker T (2003) Cytoscape: a software environment for integrated models of biomolecular interaction networks. Genome Res 13: 2498-2504
Simpson S D, Nakashima K, Narusaka Y, Seki M, Shinozaki K, Yamaguchi-Shinozaki K (2003) Two different novel cis-acting elements of erdl, a clpA homologous Arabidopsis gene function in induction by dehydration stress and dark-induced senescence. Plant J 33: 259-270
Somerville C, Browse J (1991) Plant lipids: metabolism, mutants, and membranes. Science 252: 80-87
Somssich I E, Bollmann J, Hahlbrock K, Kombrink E, Schulz W (1989) Differential early activation of defense-related genes in elicitor-treated parsley cells. Plant Mol Biol 12: 227-234
Szklarczyk D, Morris J H, Cook H, Kuhn M, Wyder S, Simonovic M, Santos A, Doncheva N T, Roth A, Bork P, Jensen L J, von Mering C (2017) The STRING database in 2017: quality-controlled protein-protein association networks, made broadly accessible. Nucleic Acids Res 45: D368
Tao F, Zhu S W, Fan J, Cheng B J (2006) Cloning and sequence analysis of maize FAD2 gene. Zhi Wu Sheng Li Yu Fen Zi Sheng Wu Xue Xue Bao 32: 649-656
Teixeira M C, Coelho N, Olsson M E, Brodelius P E, Carvalho I S, Brodelius M (2009) Molecular cloning and expression analysis of three omega-6 desaturase genes from purslane (Portulaca oleracea L.). Biotechnol Lett 31: 1089-1101
Thambugala D, Duguid S, Loewen E, Rowland G, Booker H, You F M, Cloutier S (2013) Genetic variation of six desaturase genes in flax and their impact on fatty acid composition. Theor Appl Genet 126: 2627-2641
Upchurch R G (2008) Fatty acid unsaturation, mobilization, and regulation in the response of plants to stress. Biotechnol Lett 30: 967-977
Uppalapati S R, Ishiga Y, Ryu C M, Ishiga T, Wang K, Noel L D, Parker J E, Mysore K S (2011) SGT1 contributes to coronatine signaling and Pseudomonas syringae pv. tomato disease symptom development in tomato and Arabidopsis. New Phytol 189: 83-93
USDA NASS (USDA, National Agricultural Statistics Service) 2018. Vegetables 2017 Summary. Retrieved Mar. 8, 2019, from nass.usda.gov/Publications.
van de Loo, F. J., Broun P, Turner S, Somerville C (1995) An oleate 12-hydroxylase from Ricinus communis L. is a fatty acyl desaturase homolog. Proc Natl Acad Sci USA 92: 6743-6747
Velasquez A C, Chakravarthy S, Martin G B (2009) Virus-induced gene silencing (VIGS) in Nicotiana benthamiana and tomato. J Vis Exp (28). pii: 1292. doi: 10.3791/1292
Wallis J G, Browse J (2002) Mutants of Arabidopsis reveal many roles for membrane lipids. Prog Lipid Res 41: 254-278
Wang X, Beno-Moualem D, Kobiler I, Leikin-Frenkel A, Lichter A, Prusky D (2004) Expression of Delta(12) fatty acid desaturase during the induced accumulation of the antifungal diene in avocado fruits. Mol Plant Pathol 5: 575-585
Wang Y, Zhang X, Zhao Y, Prakash C S, He G, Yin D (2015) Insights into the novel members of the FAD2 gene family involved in high-oleate fluxes in peanut. Genome 58: 375-383
Wasternack C, Stenzel I, Hause B, Hause G, Kutter C, Maucher H, Neumerkel J, Feussner I, Miersch O (2006) The wound response in tomato—role of jasmonic acid. J Plant Physiol 163: 297-306
Weber H (2002) Fatty acid-derived signals in plants. Trends Plant Sci 7: 217-224
WPTC (The World Processing Tomato Council). (2019). World production estimate as of 12 Feb. 2019. Retrieved Mar. 8, 2019, from wptc.to/releases-wptc.php
Wu C, Jia L, Goggin F (2011) The reliability of virus-induced gene silencing experiments using tobacco rattle virus in tomato is influenced by the size of the vector control. Mol Plant Pathol 12: 299-305
Yang P, Li X, Shipp M J, Shockey J M, Cahoon E B (2010) Mining the bitter melon (Momordica charantia 1.) seed transcriptome by 454 analysis of non-normalized and normalized cDNA populations for conjugated fatty acid metabolism-related genes. BMC Plant Biol 10: 250
Yara A, Yaeno T, Hasegawa M, Seto H, Montillet J L, Kusumi K, Seo S, Iba K (2007) Disease resistance against Magnaporthe grisea is enhanced in transgenic rice with suppression of omega-3 fatty acid desaturases. Plant Cell Physiol 48: 1263-1274
Yi X, Du Z, Su Z (2013) PlantGSEA: a gene set enrichment analysis toolkit for plant community. Nucleic Acids Res 41: 98
Zhang D, Pirtle I L, Park S J, Nampaisansuk M, Neogi P, Wanjie S W, Pirtle R M, Chapman K D (2009) Identification and expression of a new delta-12 fatty acid desaturase (FAD2-4) gene in upland cotton and its functional expression in yeast and Arabidopsis thaliana plants. Plant Physiol Biochem 47: 462-471
Zhang J T, Zhu J Q, Zhu Q, Liu H, Gao X S, Zhang H X (2009) Fatty acid desaturase-6 (Fad6) is required for salt tolerance in Arabidopsis thaliana. Biochem Biophys Res Commun 390: 469-474
Zhang J, Liu H, Sun J, Li B, Zhu Q, Chen S, Zhang H (2012) Arabidopsis fatty acid desaturase FAD2 is required for salt tolerance during seed germination and early seedling growth. PLoS One 7: e30355
Zoeller M, Stingl N, Krischke M, Fekete A, Waller F, Berger S, Mueller M J (2012) Lipid profiling of the Arabidopsis hypersensitive response reveals specific lipid peroxidation and fragmentation processes: biogenesis of pimelic and azelaic acid. Plant Physiol 160: 365-378
Zuorro A, Lavecchia R, Medici F, Piga L (2013) Enzyme-Assisted Production of Tomato Seed Oil Enriched with Lycopene from Tomato Pomace. Food and Bioprocess Technology 6: 3499-3509

We claim:

1. An engineered tomato plant with increased biotic stress tolerance, the engineered tomato plant comprising a non-natural modification that increases the function of a target gene selected from the group consisting of Solanum lycopersicum Fatty Acid Desaturase 2-3 (SlFAD2-3), Solanum lycopersicum Fatty Acid Desaturase 2-4 (SlFAD2-4), Solanum lycopersicum Fatty Acid Desaturase 2-5 (SlFAD2-5), Solanum lycopersicum Fatty Acid Desaturase 2-6 (SlFAD2-6), Solanum lycopersicum Fatty Acid Desaturase 2-7 (SlFAD2-7), and Solanum lycopersicum Fatty Acid Desaturase 2-9 (SlFAD2-9), wherein the engineered tomato plant exhibits increased biotic stress tolerance as compared to a tomato plant lacking said modification.

2. The engineered tomato plant of claim 1, wherein the non-natural modification increases the function of the SlFAD2-4 gene.

3. The engineered tomato plant of claim 1, wherein the non-natural modification increases the function of the SlFAD2-7 gene.

4. The engineered tomato plant of claim 1, wherein the biotic stress tolerance is selected from the group consisting of a bacteria, a fungi, a virus, an oomycetes, a viroid, a nematode, or an insect.

5. The engineered tomato plant of claim 1, wherein the engineered tomato plant is derived from a tomato cultivar selected from the group consisting of Moneymaker, MicroTom, Castlemart, Ailsa Craig, Trust, Hybrid 882, Yaqui, Monica, Mountain Belle, and BHN 444.

6. The engineered tomato plant of claim 1, wherein the engineered tomato plant comprises a vector comprising the target gene and a promoter.

7. The engineered tomato plant of claim 6, wherein the promoter is selected from the group consisting of Cauliflower Mosaic Virus 35S promoter, Figwort Mosaic Virus 34S promoter, Arabidopsis Ubiquitin10 promoter, 4×RSRE, sab, 4×GCC, CAB promoters, SlREO promoter, E273715, E541096, napin, legumin, and USP.

8. An engineered tomato plant with decreased polyunsaturated fatty acid production, the engineered tomato plant comprising a non-natural modification that decreases or eliminates the function of a target gene selected from the group consisting of Solanum lycopersicum Fatty Acid Desaturase 2-1 (SlFAD2-1), Solanum lycopersicum Fatty Acid Desaturase 2-2 (SlFAD2-2), and combinations thereof, wherein the engineered tomato plant exhibits decreased polyunsaturated fatty acid production, increased monounsaturated fatty acid production, increased heat tolerance, or combinations thereof compared to a tomato plant lacking said modification.

9. The engineered tomato plant of claim 8, wherein the non-natural modification decreases the function of the SlFAD2-1 gene.

10. The engineered tomato plant of claim 8, wherein the non-natural modification decreases the function of the SlFAD2-2 gene.

11. The engineered tomato plant of claim 8, wherein the engineered tomato plant is derived from a tomato cultivar selected from the group consisting of Moneymaker, MicroTom, Castlemart, Ailsa Craig, Trust, Hybrid 882, Yaqui, Monica, Mountain Belle, and BHN 444.

12. An engineered tomato plant with decreased linoleic acid production, the engineered tomato plant comprising a non-natural modification that silences a target gene selected from the group consisting of Solanum lycopersicum Fatty Acid Desaturase 2-1 (SlFAD2-1), and Solanum lycopersicum Fatty Acid Desaturase 2-2 (SlFAD2-2), wherein the engineered tomato plant exhibits decreased linoleic acid production, increased oleic acid production, increased stress tolerance, or combinations thereof compared to a tomato plant lacking said modification.

13. The engineered tomato plant of claim 12, wherein the SlFAD2-1 gene is silenced.

14. The engineered tomato plant of claim 12, wherein the SlFAD2-2 gene is silenced.

15. The engineered tomato plant of claim 12, wherein the engineered tomato plant is derived from the Castlemart tomato cultivar.

16. The engineered tomato plant of claim 12, wherein the engineered tomato plant comprises a vector encoding an RNA specific to the target gene selected from the group consisting of microRNA, small-interfering RNA, hairpin RNA, double-stranded RNA, and combinations thereof operably linked to a tomato specific promoter.

17. The engineered tomato plant of claim 16, wherein the promoter is selected from the group consisting of Cauliflower Mosaic Virus 35S promoter, Figwort Mosaic Virus 34S promoter, Arabidopsis Ubiquitin10 promoter, 4×RSRE, sab, 4×GCC, CAB promoters, SlREO promoter, E273715, E541096, napin, legumin, and USP.

18. The engineered tomato plant of claim 12, wherein the function of the target gene is silenced using CRISPR/Cas mediated gene silencing.

19. The engineered tomato plant of claim 18, wherein the CRISPR/Cas mediated gene silencing is under the control of a tomato specific promoter.

20. The engineered tomato plant of claim 19, wherein the promoter is selected from the group consisting of Cauliflower Mosaic Virus 35S promoter, Figwort Mosaic Virus 34S promoter, Arabidopsis Ubiquitin10 promoter, 4×RSRE, sab, 4×GCC, CAB promoters, SlREO promoter, E273715, E541096, napin, legumin, and USP.

21. The engineered tomato plant of claim 20, wherein the promoter is a seed specific promoter selected from the group consisting of E273715, E541096, napin, legumin, and USP.

22. A seed of the engineered tomato plant of claim 8, wherein the seed comprises a lower concentration of polyunsaturated fatty acid than a seed from a tomato plant lacking said non-natural modification or the seed comprises a higher concentration of monounsaturated fatty acid than a seed from a tomato plant lacking said non-natural modification.

23. A seed of the engineered tomato plant of claim 12, wherein the seed comprises a lower concentration of polyunsaturated fatty acid than a seed from a tomato plant lacking said non-natural modification or the seed comprises a higher concentration of monounsaturated fatty acid than a seed from a tomato plant lacking said non-natural modification.

23. The engineered tomato plant of claim 12, wherein the engineered tomato plant has increased stress tolerance compared to a tomato plant lacking said modification.

24. A seed of the engineered tomato plant of claim 1, wherein the engineered tomato plant exhibits increased biotic stress tolerance as compared to a tomato plant lacking said modification.


SEQUENCE LISTING (66 sequences)

SEQ ID NO: 1 - DNA, 24 bp, Artificial Sequence
  Synthetic- primer for cloning SlFAD gene
caccatgggagctggtggtcgtat

SEQ ID NO: 2 - DNA, 20 bp, Artificial Sequence
  Synthetic- primer for cloning SlFAD gene
tcagagcttgtttttgtacc

SEQ ID NO: 3 - DNA, 25 bp, Artificial Sequence
  Synthetic- primer for cloning SlFAD gene
caccatgtccactccttctgagggc

SEQ ID NO: 4 - DNA, 23 bp, Artificial Sequence
  Synthetic- primer for cloning SlFAD gene
ctaatgaaacttgtttttatacc

SEQ ID NO: 5 - DNA, 26 bp, Artificial Sequence
  Synthetic- primer for cloning SlFAD gene
caccatgggaggtggtggtaatatga

SEQ ID NO: 6 - DNA, 23 bp, Artificial Sequence
  Synthetic- primer for cloning SlFAD gene
ttagagtttgtttttataccaaa

SEQ ID NO: 7 - DNA, 26 bp, Artificial Sequence
  Synthetic- primer for cloning SlFAD gene
caccatgggaggtggtggtaatatga

SEQ ID NO: 8 - DNA, 21 bp, Artificial Sequence
  Synthetic- primer for cloning SlFAD gene
ttagagtttgtttttatacca

SEQ ID NO: 9 - DNA, 27 bp, Artificial Sequence
  Synthetic- primer for cloning SlFAD gene
caccatggacaacgatatatcatccga

SEQ ID NO: 10 - DNA, 22 bp, Artificial Sequence
  Synthetic- primer for cloning SlFAD gene
ctatattccagtagaaacacaa

SEQ ID NO: 11 - DNA, 27 bp, Artificial Sequence
  Synthetic- primer for cloning SlFAD gene
caccatgggaggtggtggtaatatgtc

SEQ ID NO: 12 - DNA, 22 bp, Artificial Sequence
  Synthetic- primer for cloning SlFAD gene
tcaaagcttgtttttgtaccaa

SEQ ID NO: 13 - DNA, 27 bp, Artificial Sequence
  Synthetic- primer for cloning SlFAD gene
caccatgggaggtggtggtaatatgtc

SEQ ID NO: 14 - DNA, 21 bp, Artificial Sequence
  Synthetic- primer for cloning SlFAD gene
tcaaatattgtttttgtacca

SEQ ID NO: 15 - DNA, 27 bp, Artificial Sequence
  Synthetic- primer for cloning SlFAD gene
caccatgttgtctgataacaagaaaaa

SEQ ID NO: 16 - DNA, 22 bp, Artificial Sequence
  Synthetic- primer for cloning SlFAD gene
ttaattcatctcacttttatac

SEQ ID NO: 17 - DNA, 27 bp, Artificial Sequence
  Synthetic- primer for cloning SlFAD gene
caccatgggaggtggtggtaattctat

SEQ ID NO: 18 - DNA, 22 bp, Artificial Sequence
  Synthetic- primer for cloning SlFAD gene
ttatgtgttgtaatttgtatac

SEQ ID NO: 19 - DNA, 28 bp, Artificial Sequence
  Synthetic- primer for cloning SlFAD gene
caccatggcttgcagtgttgcagactct

SEQ ID NO: 20 - DNA, 22 bp, Artificial Sequence
  Synthetic- primer for cloning SlFAD gene
tcaagcgtagtcaggcattact

SEQ ID NO: 21 - DNA, 20 bp, Artificial Sequence
  Synthetic- primer for detection of SlFAD gene by RT-qPCR
tccttgtcctgatcacctac

SEQ ID NO: 22 - DNA, 20 bp, Artificial Sequence
  Synthetic- primer for detection of SlFAD gene by RT-qPCR
gagatgattcgtctttctcg

SEQ ID NO: 23 - DNA, 20 bp, Artificial Sequence
  Synthetic- primer for detection of SlFAD gene by RT-qPCR
gttgtccagctcactctagg

SEQ ID NO: 24 - DNA, 20 bp, Artificial Sequence
  Synthetic- primer for detection of SlFAD gene by RT-qPCR
tgtcataatgaggcaatgaa

SEQ ID NO: 25 - DNA, 22 bp, Artificial Sequence
  Synthetic- primer for detection of SlFAD gene by RT-qPCR
tggaaacgtactcatcatcgtc

SEQ ID NO: 26 - DNA, 25 bp, Artificial Sequence
  Synthetic- primer for detection of SlFAD gene by RT-qPCR
tgaaaaacgttagttagtataccaa

SEQ ID NO: 27 - DNA, 20 bp, Artificial Sequence
  Synthetic- primer for detection of SlFAD gene by RT-qPCR
gaaggctattcctcctcatt

SEQ ID NO: 28 - DNA, 20 bp, Artificial Sequence
  Synthetic- primer for detection of SlFAD gene by RT-qPCR
acgacgatgactgtatttcc

SEQ ID NO: 29 - DNA, 20 bp, Artificial Sequence
  Synthetic- primer for detection of SlFAD gene by RT-qPCR
tggacaacgatatatcatcc

SEQ ID NO: 30 - DNA, 20 bp, Artificial Sequence
  Synthetic- primer for detection of SlFAD gene by RT-qPCR
accttgagcaatcgagtaaa

SEQ ID NO: 31 - DNA, 20 bp, Artificial Sequence
  Synthetic- primer for detection of SlFAD gene by RT-qPCR
cttcaaagcctccttttaca

SEQ ID NO: 32 - DNA, 20 bp, Artificial Sequence
  Synthetic- primer for detection of SlFAD gene by RT-qPCR
gtggaggataagaccaacaa

SEQ ID NO: 33 - DNA, 20 bp, Artificial Sequence
  Synthetic- primer for detection of SlFAD gene by RT-qPCR
actgcttcccttgagaacga

SEQ ID NO: 34 - DNA, 20 bp, Artificial Sequence
  Synthetic- primer for detection of SlFAD gene by RT-qPCR
gctcctcgtagccaattcca

SEQ ID NO: 35 - DNA, 20 bp, Artificial Sequence
  Synthetic- primer for detection of SlFAD gene by RT-qPCR
atggctttgcaagtcactat

SEQ ID NO: 36 - DNA, 20 bp, Artificial Sequence
  Synthetic- primer for detection of SlFAD gene by RT-qPCR
gaatgtggtggaagacctta

SEQ ID NO: 37 - DNA, 20 bp, Artificial Sequence
  Synthetic- primer for detection of SlFAD gene by RT-qPCR
accccttgtaattgtgaatg

SEQ ID NO: 38 - DNA, 20 bp, Artificial Sequence
  Synthetic- primer for detection of SlFAD gene by RT-qPCR
agatgcttcatctttctcca

SEQ ID NO: 39 - DNA, 20 bp, Artificial Sequence
  Synthetic- primer for detection of SlFAD gene by RT-qPCR
tgttggaacactagcctttt

SEQ ID NO: 40 - DNA, 20 bp, Artificial Sequence
  Synthetic- primer for detection of SlFAD gene by RT-qPCR
tccaaaaatgataacccaac

SEQ ID NO: 41 - DNA, 20 bp, Artificial Sequence
  Synthetic- primer for detection of the control gene ribosomal protein L2 (RPL2) by RT-qPCR
gagggcgtactgagaaacca

SEQ ID NO: 42 - DNA, 20 bp, Artificial Sequence
  Synthetic- primer for detection of the control gene ribosomal protein L2 (RPL2) by RT-qPCR
cttttgtccaggaggtgcat

SEQ ID NO: 43 - DNA, 29 bp, Artificial Sequence
  Synthetic- primer for amplifying SlFAD2-7 fragment for gene silencing construct
cgggaattcactgcttcccttgagaacga

SEQ ID NO: 44 - DNA, 29 bp, Artificial Sequence
  Synthetic- primer for amplifying SlFAD2-7 fragment for gene silencing construct
cggctcgaggctcctcgtagccaattcca

SEQ ID NO: 45 - DNA, 1552 bp, Solanum lycopersicum
  misc_feature (1)..(1552): SlFAD2-1 cDNA
tgccaatgatggtttgatctctaaatcaagaccttacatgaaaggtagattaggggggtcacaccttgtttggtttgggattgaagaggcagcgtagcgaaaatgggagctggtggtcgtatgtctgctccaaatggcgggactgaagtaaagaagaatcctcttcaaaaggtaccaacctcgaagccccctttcacagttggtgatattaagaaggctatcccacctcactgctttcaaaggtctctcatccgctcattttcatatgttgtgtatgacctcatactcgtatccatcatgtactacgttgcaaacacttacttccacctcattccatcaccatattgctacattgcatggcctatttactggatttgccagggttgtgtttgcactggaatttgggttaatgcccacgaatgtggccatcacgccttcagtgattaccaattggttgatgacaccgtaggacttatccttcactctgctctattggtgccgtacttctcttggaaatatagtcatcgtcgccaccactccaacactggctccctcgagcgtgatgaagtctttgtgcctaagtctaaatcccagctcggatggtattccaagtacttgaacaatccactaggcagagttatcacactcacagtcaccctcactcttggttggccgttgtacttggcattcaatgtatctggcagaccttatgaccgatttgcatgtcactatgacccttatggcccaatctacaacaaccgcgagaggctacagatcttcctttctgatgctggagttctcggagcttgttatctgttataccgtgttgccttagtgaaaggtctagcttggctagtgtgcatctacggtgtgccccttctagtcgtgaacggcttccttgtcctgatcacctacttgcagcacactcacccatcactgcctcactacgactcaaccgagtgggattggcttaggggagctttggcaacctgtgacagagactatggtgttctaaacaaggtcttccacaacatcaccgacactcacgtggtgcaccatctgttctcagccatgccacactacaacgcgatggaggcaaccaaagcagtcaagccactactcggagactactaccaattcgatggtacccccattttcaaggcaatgtggagggaagctaaagagtgtctctacgtcgagaaagacgaatcatctcaaggcaaaggtgtcttctggtacaaaaacaagctctgagaacactagaacagaaactcagcttgctttgtattggtcttttggtactgtttggtctctctgtgtcttaagtattttggatttaagttaatttagctaatgtgttgtctttgctagtgaggggaggtttatgtttgatgaattttttttgataaatcaaaggacaaaaaatttagaaaaatgtaaagaggatcaaaatgcatcctccacctcctcctagtatgtaataaactaccagttttaatataaatggttgtgtttgtgtctacaattggattagaagcattctttcgattgc

SEQ ID NO: 46 - DNA, 1137 bp, Solanum lycopersicum
  misc_feature (1)..(1137): SlFAD2-2 cDNA
atgtccactccttctgagggcaacaagtctcagtctaatgtcctccaaagagttcctcattcgaaaccacccttcactgtgggtgacgtcaagaaagccatcccaccccattgtttcaagcgatctgtcctacactctttctccttcgttctttatgacctcgtagtagcctttctcctttactatgttgcaactaactatttccatctcctcccttacaatttatcctacgttgcctggccgctttactggatctgccaaggatgtaatctaacaggagtttgggtcatagcacatgaatgtggtcatcatgccttcagtgactatcaatggcttgatgacactgttggccttgtcctacactcatcactacttgttccctatttctcttggaaatatagtcatcgtcgtcatcattccaatacaggttcaatggatcgtgatgaagtatttgtaccaaagaagaagtcgagtatgaaatggttctctacttatctcaacaactcaccaggaagaattcttgtacttgttgtccagctcactctaggctggcctttatacctcatgtttaatgtttcaggtagaccttatgatcggtttgcttgccatttcgatcccaatagccctatctacacagatcgtgaacgccttcagatctttgtatctgatgctggcatatttgctgtactctatgtactttacactcttgtggcagcaaaaggacttgcctgggttgtctgtgtttatggatgtccattactcattgtcaatggattcctcgtattgataacatatttacaacacacacatccttcattgcctcattatgacacatcggaatgggactggttgaggggtgctcttgctacagttgacagagactatggaattctcaacaaggtattccataacatcacagacactcatgtagcacaccatctattttcaacgatgccgcattatcatgcaatggaagctacaaaggctataaaaccaatattaggtgactattatcaatttgatgggacatcaatttggaaggccatgtatagagaagctaaagagtgtgtttacgtcgagccagatgaaggtgaccaaaacaagggtgtcttttggtataaaaacaagtttcattag

SEQ ID NO: 47 - DNA, 1128 bp, Solanum lycopersicum
  misc_feature (1)..(1128): SlFAD2-3 cDNA
atgggaggtggtggtaatatgactaaaattgaacaaaagattaaagtaccactattaaagcccccttttaaacttagtgaactcaagaaggctattcctcctcactgctttcaacgatctctcgttcgctccttctcctacgttgttcgtgatatcacactagtgtttgtattagggtatatcgctgcgaattacatctatgtccttccatccccatattattacctagcatggcctatttattggtttgctctaggtagtgctttcacaggaatatgggtccttgctcatgaatgtggccaccatggctttagtaattatcaatggatagatgacacggttggttttatcttatatacctcgatttgtgtgccttacttttcttggaaacgtactcatcatcgtcatcactccaatactggttcccttgaatatgatgaagtctatgtacctaggcttaaatccgaactacaatggttctccaaatatttcaacaatctaccaggacgaataatcgcgttcaccaccacccttacggttggttggccttcttacatggccatcaacgcctccggtagaccttatgatcgttttgcaagtcactataatccatatagtccaatgtataatgatagtgagaggctactaatatacttgtcagacttgggtctaatttcaatgatttatatgtggtatcgaatcgcgatggtaaaagggctagcttgggttgtatgcatgtatgtagcgccccttcaaataatgagcatactagtagtggtactaacttatttgaaccacacacatgcattagtcccacattatgattcttcagaatgggattggctaagaggaactctagctactgtagatagagactttggtatactaactaacgtttttcataacgttacagatactcatgttttgcatcatttattcacaaccataccacattatcatgcaatggaggcaacaaaagctatcaagccaatacttggagattactataattttgatagtacaccagtttataaagcgatatggaagaatataaatgagtgcatctatgtggaaaaaaaggaagaaactcaagatagaggtgttttttggtataaaaacaaactctaa

SEQ ID NO: 48 - DNA, 1128 bp, Solanum lycopersicum
  misc_feature (1)..(1128): SlFAD2-4 cDNA
atgggaggtggtggtaatatgactaaaattgaacaaaagattaaagtaccactatccaagcccccttttatacttagtgaactcaagaaggctattcctcctcattgctttcaacgatctctcgttcgctccttttcctacgttgttcgggatatcacactagtgtttgtattagggtatatcgctgcgaattacatctatgtccttccatctccatataattacctaggatggcctatttattggttttccctaggaagtgttttcacagcaatgtgggtcattgctcatgaatgtggccatcatggctttagtaattaccaatggatagatgacacggttggttttatcttctatacctcaatttgtgtcccttacttttcttggaaatacagtcatcgtcgtcatcactccaatgcaggttcgcttgaatatgatgaagtctacgtacctaggcttaaatccgaattacgttggttctctaaatatttcgacaatctaccaggacgaataatcgcgttcaccaccaccctcacggttggttggccttcttacatggccatcaatgcctccggtagaccttatgatcgttttgcaagtcactataatccatatagtccaatgtataacaatcgtgagaggctacttatctacttctcagacttaggtctaattgcatttatttatatgtggtatcgtatcgcgatggtaaaagggctagcttgggttgtatgtatgtatgtagcaccccttgaaattatgaacatactagtagtggtactaacttttttgaaccacacacatgcattagtcccacattatgattcttcagaatgggattggctaagaggagctttagctactgtagatagagactttggtccactaaataatatatgtcataatgttacaaatactcatgttttgcatcatttattcacaaccataccacattatcatgcagtggaggcaacaaaagctatcaagccaatacttggagattactataattttgatagtacaccagtttataaagcaatatggaagaatataaatgagtgtatctatgtggagaaaaatgaagaaactcaagatagaggtgttttttggtataaaaacaaactctaa

SEQ ID NO: 49 - DNA, 282 bp, Solanum lycopersicum
  misc_feature (1)..(282): SlFAD2-5 cDNA
atggacaacgatatatcatccgataaaacagaacaaaacaaaaatcgtgtgtcattttcaaagtttccttttacaattggcgatatcaagaaattcatccctcctcactgttttcaacgatctcttgttcgttccttctcatatcttgttcaagatctcatccctgtttccatcttttattatattgccaccatttacttccatctccttacgtctccgtgttgttacctagcatggcctatttactcgattgctcaaggttgtgtttctactggaatatag

SEQ ID NO: 50 - DNA, 951 bp, Solanum lycopersicum
  misc_feature (1)..(951): SlFAD2-6 cDNA
atgggaggtggtggtaatatgtctgctatgacaagtaaaaacgaacagaagaaaaatcctctcgaaagagtgccatcttcaaagcctccttttacacttggtgatgtaaagaaggccatccctccccattgcttcgaacgatctcttgtaaaatcattttcgtttcttatccaggatctgatactcgtctacatcttttattacatcgccaacacttacattcacctcattccaaccccatataggtatgtagcatggaccacttattggattgctcaaggttgtgtttgcaccggaatatgggtcattggccatgaatgtggtcaccatggctttagtgattaccaatgggtagatgacattgttggtcttatcctccactcagctcttctaacgccatactttgcatggaaacatagtcatcgtcgtcaccatgcaaacacaggttcccttgagaatgatgaaatttacataccaagacttaagactagcaaaaaatatgaccgttttgcatgtcattatgatccatatagcccaatatattcaaatcgcgaaaggctacaaatctacatttcagatgtaggtgtgattgcaacaacttatttattatatcgcgttactttgacacaagggctagcttggatttatagtgttgatcactcttattgggatcatctaagaggagctttagctacggtagatagagattatggtttgttaaataaagtgttccacaatgttacggatacacatgttttgcatcatatattctcatatatatcacattaccatgcaatggaggcaacaaaagctatcaaaccattgttgggagaatactacaaatatgatgatacaccaattttaaaggcaatgtggagggatacaaaggagtgtatctttgtggagaaagataaagataaaggagtttattggtacaaaaacaagctttga

SEQ ID NO: 51 - DNA, 1155 bp, Solanum lycopersicum
  misc_feature (1)..(1155): SlFAD2-7 cDNA
atgggaggtggtggtaatatgtctgttccaacaactaaaactgaacaaaagaaaaatcctcttcaaagagcgccaatttcaaagcccccttttacacttagtgacatcaagaaagccatccctcctcactgctttgagcggtctctcattcgctcatcctactatgttgtacatgatctagtattgatctacatcttctattacatcgcgtccacttacttccatacccttccatctccatactcttacctagcatggttcgcttattggatcgttcaaggttgtgtttccacaggtatatgggtcaatgcccatgaatgtggtcaccaagcgtttagtcattacccgttgataaatgacactattggttttatcctccactcagcgcttttaacaccatacttctcatggaagtatagtcatcgccgtcaccactccaatactgcttcccttgagaacgatgagaactatgttcccaaaaccaagtcagaattaaaatggttcaccaaagcatacgcgaataatccattaggacgattattcatactcgtatttaccctcaccgttgggttacctttatactatgccataaatgtggctggaagaccttatgatcgctttgcaagtcattataatccatatagccctatatataacaatcgtgaaaggctacaaatttacatttctgatataggtgtgattgcaactagttatgtattgtatcgcgttgcatgcacacaagggctaacttggcttgtatgcatctatggagtacccctcctaattgtaaacggattcatagtgttgatcacgtttttgcaccatacacactcttcattaccacattacgattcacaagaatggaattggctacgaggagctctagctacagtagatagagactatggtgtgttaaattacttcttccataacattgcggatactcatgttatgcatcatctattctcatcaattccacattaccatgcaatagaggcaacaaaagctatcaagccagtattaggagaatactaccaatatgatggtaccccaatttataaggcaatgtggagggacttcaaggagtgcatctatgttgagaaagataatgaatctcaagaaaaaggtgtttattggtacaaaaacaatatttga

SEQ ID NO: 52 - DNA, 1107 bp, Solanum lycopersicum
  misc_feature (1)..(1107): SlFAD2-8 cDNA
atgttgtctgataacaagaaaaatggagtgtcatttgcaaagcccccttttacaattggcgatatcaagaaggccatccctcctcactgctttcaacgatctcttgttcgttctttctcctatcttgttcaagatctcgtccttgtctccgtcttttattatattgccaccacttacttccaccctctattttgttacctagcatggcctgtttactggattgctcaaggttgtgttttaactggaatatggatgattggccatgaattaggccaccatgccttcagtgattacccgtggataaatgacactattggttttatcctccactcttcacttttaacaccatacttctcatggaaatatactcatcgtcgtcaccactccaacactagttcccttgagcatgatgaagtgtatgttccaagactaaaatcccaagtaacgtggctcaccaagtatttgaacaaaaatccactaggacgagtattcggacttgccaccaccctcaatctagggtggcctttgtatttggccttcaatgcctctggcagaccatatcatggctttgcaagtcactatcatccacatggaccgagttattatgatcgcgagaggctacaaatctatttttcagatgcaggtgtgattgcaacaacttatgtgttgtatcgcattgctttggcacaagggctaacttggcttgtgtgtatctatggagtgcccctccaaattatgaacatcttcatagtgttaatcactctcttgaaccatactcactcttccgtgccacattatgattcatcagagtgggattggctaagaggagctctagctacggtagacagagactttggtgtactaaataaggtcttccaccacattccggatgctcacgtcttgcatcatctattctcaacgattccacattaccacgcactcgaggcaaccagagctatcaagccattactaggagaatactaccaatttgatagtacccctttttataaggcattatggagggattataaggagtgcatctatgtggaaaaggatgaaagatctaaggacagcggtatttattggtataaaagtgagatgaattaa

SEQ ID NO: 53 - DNA, 1140 bp, Solanum lycopersicum
  misc_feature (1)..(1140): SlFAD2-9 cDNA
atgggaggtggtggtaattctatgagtaatccaacaactaaaaatcgagttccaagttcaaagcccccttttacgattggtgatatcaagaaggctatccctcctcattgctttcaacgctctctcgttcgctcctcttcctatcttatttacgatctcatacttgtcttcatcttctattacatcgcctctacttatttccacgtccttccaaaaccgtattatccatacctagcatggcctatttattggatcgttcaaggatgtgtttgcaccgcactatgggtcattgcccatgaatgtggccaccaatcctttagtgattaccaatggataaatgacactatcggttttatcctccactctgcacttttaacaccatacttctcatggaaatatagtcatcgtcgtcatcactccaatactaattcccttgagcatgatgaaaatcatgtgcccaaacttgaagtcaaactaaaatggtacactaaattatacgtgaataatccacttggacgattattattaattgtcttcacactcactgctggcttacctttatactatgccatcaatatagccggtagaccttatgatcgttttgcaagtcattatgatccatatagccctatctacaatgaccgtgagagactacaaatttacatctcagatgcaggtgtgattgcaactatttatttattatatcgcgttgctctaacacaaggcctaacttgggttatatgcatctatggagtaccccttgtaattgtgaatgggttcatagtgttaataactttactgcatcacactcatgcttcattgccacattatgattcatcagagtggagttggctaaaaggagctctagctacggtagatagagactatggtgtcctaaataaggtgtttcatcatatcgtagatacacacgttctacatcatctattttcaagtataccacattatcatgctgtcgaggcaaccaaggccatcaagccgttactaggagaatactaccaatttgatggaactccattttacaaagcaatatggagggattttaatgagtgtcaatatgtggagaaagatgaagcatctcaacatcagggtattttctggtatacaaattacaacacataa

SEQ ID NO: 54 - DNA, 1845 bp, Solanum lycopersicum
  misc_feature: SlFAD6 cDNA
ataggccctaatagcggcaaataagttttttttgggcctttctcctcctttcaccctctatcttcaataaagaaccaatttttatgataaaagaatcaatttttacagtaaagaaccaatttttattggtttaagaagaggtgaggggtgagaagagaagggtaaagaaccataaaatctaagtggggtgttgtttttttgtaagttcttgttctgcaaatttctctctataatggcttgcagtgttgcagactctggtttcctggtcttggcttccaagaaaggaccagttcatagcactagaattgggcaaattgcttcttcaggagcatgttgtttaaagtgggaaagtctacctcagataagctcgaaacagaaaggctgcttaatttactcgagaaagaaaaatgttatacaagctgttgccgttccaatagcacctgcacccttagactctgcagaggaaaggaatcgattaagtgaaaaatatggttttagacaaattggagaaccactacctgataacgttacaatgagagatattactgagtcactaccaaagaaggtatttgagattgacgatgggaaggcattgaagtctgttttgatctcagtaacttcatacacactggggctcttcatgattgccaaagctccatggtatctacttcccctggcctgggcttggacaggaacagcagttacagggttttttgttataggacatgattgcgctcacaaatccttctcaaagaacaaattattggaagatattgttggaacactagcctttttgccattgatatatccctatgagccatggaggttcaagcatgatcggcatcacgcaaaaacaaacatgttgcacgaagacacagcttggcagcctgtttggaaggaggagttcgattcagtccctgccctgaggaaggcaattatattaggttacggccctattcgaccttggatgtctatagctcactggtggctctgtcatttcaatttgagtaagttcagaccaaatgaaattgacagagtgaaaataagtttgggttgcgtcttcgcttttatggcaatcggctggccactgataatctggaaaacagggattattggatggatcaagttctggttaatgccatggttgggttatcatttttggatgagtacttttacaatggttcatcatacggctccccacatacccttcaaaacttctgatgagtggaatgctgctcaggcccagctgaatggcacagttcactgtgactatcctagttggattgaggttctttgtcatgatatcaatgttcatattccccaccacatatcccccagaataccaagttataatttacgggcagctcatcaatctctccaagagaattggggaaagtaccttaacgaggcttcatggaactggcgtctaatgaagacaatactgacagtatgtcatgtctatgataaggagcggaattatgttccatttgatgaaattgtccccgaagaagccaagccaatcacattccttaaaaaagtaatgcctgactacgcttgattagcgtgcacctcaaaaggtcaattttcacttgcttcattctttgggaagatatcctttcaatcctattgggtccggtcaagggcccaaatgtcaagttctcacaaatattgtaagacttgttttaacacattaaagagagaaatatcttctcaaaaagtttcttggtagtgttagtttagtatacaataatttaactttcttgcgatttgtcttctttttttagaagtatccatgaataacatgttgcttattaaataccaaagcctcgttatgatcttcaacct

SEQ ID NO: 55 - protein, 383 aa, Arabidopsis thaliana
  MISC_FEATURE (1)..(383): AtFAD2 (At3g12120)
MGAGGRMPVPTSSKKSETDTTKRVPCEKPPFSVGDLKKAIPPHCFKRSIPRSFSYLISDIIIASCFYYVATNYFSLLPQPLSYLAWPLYWACQGCVLTGIWVIAHECGHHAFSDYQWLDDTVGLIFHSFLLVPYFSWKYSHRRHHSNTGSLERDEVFVPKQKSAIKWYGKYLNNPLGRIMMLTVQFVLGWPLYLAFNVSGRPYDGFACHFFPNAPIYNDRERLQIYLSDAGILAVCFGLYRYAAAQGMASMICLYGVPLLIVNAFLVLITYLQHTHPSLPHYDSSEWDWLRGALATVDRDYGILNKVFHNITDTHVAHHLFSTMPHYNAMEATKAIKPILGDYYQFDGTPWYVAMYREAKECIYVEPDREGDKKGVYWYNNKL

SEQ ID NO: 56 - protein, 383 aa, Solanum lycopersicum
  MISC_FEATURE (1)..(383): SlFAD2-1
MGAGGRMSAPNGGTEVKKNPLQKVPTSKPPFTVGDIKKAIPPHCFQRSLIRSFSYVVYDLILVSIMYYVANTYFHLIPSPYCYIAWPIYWICQGCVCTGIWVNAHECGHHAFSDYQLVDDTVGLILHSALLVPYFSWKYSHRRHHSNTGSLERDEVFVPKSKSQLGWYSKYLNNPLGRVITLTVTLTLGWPLYLAFNVSGRPYDRFACHYDPYGPIYNNRERLQIFLSDAGVLGACYLLYRVALVKGLAWLVCIYGVPLLVVNGFLVLITYLQHTHPSLPHYDSTEWDWLRGALATCDRDYGVLNKVFHNITDTHVVHHLFSAMPHYNAMEATKAVKPLLGDYYQFDGTPIFKAMWREAKECLYVEKDESSQGKGVFWYKNKL

SEQ ID NO: 57 - protein, 378 aa, Solanum lycopersicum
  MISC_FEATURE (1)..(378): SlFAD2-2
MSTPSEGNKSQSNVLQRVPHSKPPFTVGDVKKAIPPHCFKRSVLHSFSFVLYDLVVAFLLYYVATNYFHLLPYNLSYVAWPLYWICQGCNLTGVWVIAHECGHHAFSDYQWLDDTVGLVLHSSLLVPYFSWKYSHRRHHSNTGSMDRDEVFVPKKKSSMKWFSTYLNNSPGRILVLVVQLTLGWPLYLMFNVSGRPYDRFACHFDPNSPIYTDRERLQIFVSDAGIFAVLYVLYTLVAAKGLAWVVCVYGCPLLIVNGFLVLITYLQHTHPSLPHYDTSEWDWLRGALATVDRDYGILNKVFHNITDTHVAHHLFSTMPHYHAMEATKAIKPILGDYYQFDGTSIWKAMYREAKECVYVEPDEGDQNKGVFWYKNKFH

SEQ ID NO: 58 - protein, 375 aa, Solanum lycopersicum
  MISC_FEATURE (1)..(375): SlFAD2-3
MGGGGNMTKIEQKIKVPLLKPPFKLSELKKAIPPHCFQRSLVRSFSYVVRDITLVFVLGYIAANYIYVLPSPYYYLAWPIYWFALGSAFTGIWVLAHECGHHGFSNYQWIDDTVGFILYTSICVPYFSWKRTHHRHHSNTGSLEYDEVYVPRLKSELQWFSKYFNNLPGRIIAFTTTLTVGWPSYMAINASGRPYDRFASHYNPYSPMYNDSERLLIYLSDLGLISMIYMWYRIAMVKGLAWVVCMYVAPLQIMSILVVVLTYLNHTHALVPHYDSSEWDWLRGTLATVDRDFGILTNVFHNVTDTHVLHHLFTTIPHYHAMEATKAIKPILGDYYNFDSTPVYKAIWKNINECIYVEKKEETQDRGVFWYKNKL

SEQ ID NO: 59 - protein, 375 aa, Solanum lycopersicum
  MISC_FEATURE (1)..(375): SlFAD2-4
MGGGGNMTKIEQKIKVPLSKPPFILSELKKAIPPHCFQRSLVRSFSYVVRDITLVFVLGYIAANYIYVLPSPYNYLGWPIYWFSLGSVFTAMWVIAHECGHHGFSNYQWIDDTVGFIFYTSICVPYFSWKYSHRRHHSNAGSLEYDEVYVPRLKSELRWFSKYFDNLPGRIIAFTTTLTVGWPSYMAINASGRPYDRFASHYNPYSPMYNNRERLLIYFSDLGLIAFIYMWYRIAMVKGLAWVVCMYVAPLEIMNILVVVLTFLNHTHALVPHYDSSEWDWLRGALATVDRDFGPLNNICHNVTNTHVLHHLFTTIPHYHAVEATKAIKPILGDYYNFDSTPVYKAIWKNINECIYVEKNEETQDRGVFWYKNKL

SEQ ID NO: 60 - protein, 93 aa, Solanum lycopersicum
  MISC_FEATURE (1)..(93): SlFAD2-5
MDNDISSDKTEQNKNRVSFSKFPFTIGDIKKFIPPHCFQRSLVRSFSYLVQDLIPVSIFYYIATIYFHLLTSPCCYLAWPIYSIAQGCVSTGI

SEQ ID NO: 61 - protein, 316 aa, Solanum lycopersicum
  MISC_FEATURE (1)..(316): SlFAD2-6
MGGGGNMSAMTSKNEQKKNPLERVPSSKPPFTLGDVKKAIPPHCFERSLVKSFSFLIQDLILVYIFYYIANTYIHLIPTPYRYVAWTTYWIAQGCVCTGIWVIGHECGHHGFSDYQWVDDIVGLILHSALLTPYFAWKHSHRRHHANTGSLENDEIYIPRLKTSKKYDRFACHYDPYSPIYSNRERLQIYISDVGVIATTYLLYRVTLTQGLAWIYSVDHSYWDHLRGALATVDRDYGLLNKVFHNVTDTHVLHHIFSYISHYHAMEATKAIKPLLGEYYKYDDTPILKAMWRDTKECIFVEKDKDKGVYWYKNKL

SEQ ID NO: 62 - protein, 384 aa, Solanum lycopersicum
  MISC_FEATURE (1)..(384): SlFAD2-7
MGGGGNMSVPTTKTEQKKNPLQRAPISKPPFTLSDIKKAIPPHCFERSLIRSSYYVVHDLVLIYIFYYIASTYFHTLPSPYSYLAWFAYWIVQGCVSTGIWVNAHECGHQAFSHYPLINDTIGFILHSALLTPYFSWKYSHRRHHSNTASLENDENYVPKTKSELKWFTKAYANNPLGRLFILVFTLTVGLPLYYAINVAGRPYDRFASHYNPYSPIYNNRERLQIYISDIGVIATSYVLYRVACTQGLTWLVCIYGVPLLIVNGFIVLITFLHHTHSSLPHYDSQEWNWLRGALATVDRDYGVLNYFFHNIADTHVMHHLFSSIPHYHAIEATKAIKPVLGEYYQYDGTPIYKAMWRDFKECIYVEKDNESQEKGVYWYKNNI

SEQ ID NO: 63 - protein, 368 aa, Solanum lycopersicum
  MISC_FEATURE (1)..(368): SlFAD2-8
MLSDNKKNGVSFAKPPFTIGDIKKAIPPHCFQRSLVRSFSYLVQDLVLVSVFYYIATTYFHPLFCYLAWPVYWIAQGCVLTGIWMIGHELGHHAFSDYPWINDTIGFILHSSLLTPYFSWKYTHRRHHSNTSSLEHDEVYVPRLKSQVTWLTKYLNKNPLGRVFGLATTLNLGWPLYLAFNASGRPYHGFASHYHPHGPSYYDRERLQIYFSDAGVIATTYVLYRIALAQGLTWLVCIYGVPLQIMNIFIVLITLLNHTHSSVPHYDSSEWDWLRGALATVDRDFGVLNKVFHHIPDAHVLHHLFSTIPHYHALEATRAIKPLLGEYYQFDSTPFYKALWRDYKECIYVEKDERSKDSGIYWYKSEMN

SEQ ID NO: 64 - protein, 379 aa, Solanum lycopersicum
  MISC_FEATURE (1)..(379): SlFAD2-9
MGGGGNSMSNPTTKNRVPSSKPPFTIGDIKKAIPPHCFQRSLVRSSSYLIYDLILVFIFYYIASTYFHVLPKPYYPYLAWPIYWIVQGCVCTALWVIAHECGHQSFSDYQWINDTIGFILHSALLTPYFSWKYSHRRHHSNTNSLEHDENHVPKLEVKLKWYTKLYVNNPLGRLLLIVFTLTAGLPLYYAINIAGRPYDRFASHYDPYSPIYNDRERLQIYISDAGVIATIYLLYRVALTQGLTWVICIYGVPLVIVNGFIVLITLLHHTHASLPHYDSSEWSWLKGALATVDRDYGVLNKVFHHIVDTHVLHHLFSSIPHYHAVEATKAIKPLLGEYYQFDGTPFYKAIWRDFNECQYVEKDEASQHQGIFWYTNYNT

SEQ ID NO: 65 - protein, 448 aa, Arabidopsis thaliana
  MISC_FEATURE (1)..(448): AtFAD6 (At4g30950)
MASRIADSLFAFTGPQQCLPRVPKLAASSARVSPGVYAVKPIDLLLKGRTHRSRRCVAPVKRRIGCIKAVAAPVAPPSADSAEDREQLAESYGFRQIGEDLPENVTLKDIMDTLPKEVFEIDDLKALKSVLISVTSYTLGLFMIAKSPWYLLPLAWAWTGTAITGFFVIGHDCAHKSFSKNKLVEDIVGTLAFLPLVYPYEPWRFKHDRHHAKTNMLVHDTAWQPVPPEEFESSPVMRKAIIFGYGPIRPWLSIAHWVNWHFNLKKFRASEVNRVKISLACVFAFMAVGWPLIVYKVGILGWVKFWLMPWLGYHFWMSTFTMVHHTAPHIPFKPADEWNAAQAQLNGTVHCDYPSWIEILCHDINVHIPHHISPRIPSYNLRAAHESIQENWGKYTNLATWNWRLMKTIMTVCHVYDKEENYIPFDRLAPEESQPITFLKKAMPNYTA

SEQ ID NO: 66 - protein, 441 aa, Solanum lycopersicum
  MISC_FEATURE (1)..(441): SlFAD6
MACSVADSGFLVLASKKGPVHSTRIGQIASSGACCLKWESLPQISSKQKGCLIYSRKKNVIQAVAVPIAPAPLDSAEERNRLSEKYGFRQIGEPLPDNVTMRDITESLPKKVFEIDDGKALKSVLISVTSYTLGLFMIAKAPWYLLPLAWAWTGTAVTGFFVIGHDCAHKSFSKNKLLEDIVGTLAFLPLIYPYEPWRFKHDRHHAKTNMLHEDTAWQPVWKEEFDSVPALRKAIILGYGPIRPWMSIAHWWLCHFNLSKFRPNEIDRVKISLGCVFAFMAIGWPLIIWKTGIIGWIKFWLMPWLGYHFWMSTFTMVHHTAPHIPFKTSDEWNAAQAQLNGTVHCDYPSWIEVLCHDINVHIPHHISPRIPSYNLRAAHQSLQENWGKYLNEASWNWRLMKTILTVCHVYDKERNYVPFDEIVPEEAKPITFLKKVMPDYA